(19) United States
(12) Patent Application Publication
(10) Pub. No.: US 2022/0411766 A1
(43) Pub. Date: Dec. 29, 2022

(54) COMPOSITIONS AND METHODS FOR USING GENETICALLY MODIFIED ORTHOLOGOUS ENZYMES

(71) Applicant: Renew Biopharma, Inc., San Diego, CA (US)

(72) Inventors: Michael J. MENDEZ, San Diego, CA (US); Kyle BOTSCH, San Diego, CA (US); Tasha ALTHEIDE, San Diego, CA (US); Matthew SAUNDERS, San Diego, CA (US)

(21) Appl. No.: 17/765,767

(22) PCT Filed: Oct. 2, 2020
(86) PCT No.: PCT/US2020/054003

§ 371 Date: Mar. 31, 2022

Related U.S. Application Data
(60) Provisional application No. 62/910,331, filed on Oct. 3, 2019.

(51) Int. Cl. C12N 9/10

(52) U.S. Cl. CPC C12N 9/1085; C12Y 205/01102; C12N 2510/02; C12N 2800/101; C12N 2800/22

(57) ABSTRACT

Described herein are prenyltransferases including non-natural variants thereof having at least one amino acid substitution as compared to its corresponding natural or unmodified prenyltransferases and that are capable of at least two-fold greater rate of formation of cannabinoids such as cannabigerolic acid, cannabigerovarinic acid, cannabigerorcinic acid, and cannabigerol, as compared to a wild type control. Prenyltransferase variants also accept different hydrophobic substrates (e.g., “donor” molecules), compared to wild type controls, to create different minor and novel cannabinoids. Prenyltransferase variants also demonstrated regioselectivity to desired cannabinoid isomers such as CBGA (3-GOLA), 3-GDVA, 3-GOSA, and CBG (2-GOL). The prenyltransferase variants can be used to form prenylated aromatic compounds, and can be expressed in an engineered microbe having a pathway to such compounds, which include 3-GOLA, 3-GDVA, 3-GOSA, and CBG. 3-GOLA can be used for the preparation of cannabigerol (CBG), which can be used in therapeutic compositions.

### CROSS-REFERENCE TO RELATED APPLICATIONS

This application claims the benefit of U.S. Provisional Application No. 62/910,331 filed Oct. 3, 2019, which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Oct. 1, 2020, is named 56397-702_601_SL.txt and is 149,627 bytes in size.

### BACKGROUND

Cannabinoids constitute a varied class of chemicals that bind to cellular cannabinoid receptors. Modulation of these receptors has been associated with different types of physiological processes including pain-sensation, memory, mood, and appetite. Endocannabinoids, which occur in the body, phytocannabinoids, which are found in plants such as Cannabis, and synthetic cannabinoids, can have activity on cannabinoid receptors and elicit biological responses.

Cannabis sativa produces a variety of phytocannabinoids, the most notable of which is a precursor of tetrahydrocannabinol (THC), the primary psychoactive compound in cannabis. However, C. sativa also produces precursors of other cannabinoids such as cannabidiol (CBD), cannabigerol (CBG), and cannabichromene (CBC). CBD, CBG, and CBC, unlike THC, are not psychoactive. In C. sativa, precursors of CBD, CBG, CBC, and THC, are carboxylic acid-containing molecules referred to as Δ⁹-tetrahydrocannabinoic acid (Δ⁹-THCA), CBDA, cannabigerolic acid (CBGA), and cannabichromenic acid (CBCA), respectively. Δ⁹-THCA, CBDA, CBGA, and CBCA are bioactive after decarboxylation, such as caused by heating, to their bioactive forms (e.g. CBGA to CBG).

Despite the well-known actions of THC, the non-psychoactive CBD, CBG, and CBC cannabinoids also have important therapeutic uses. For example, these cannabinoids can be used for the treatment of conditions and diseases that are altered or improved by action on the CB₁ and/or CB₂ cannabinoid receptors, and/or a2-adrenergic receptor. CBG has been proposed for the treatment of glaucoma as it has been shown to relieve intraocular pressure. CBG can also be used to treat inflammatory bowel disease. Further, CBG can also inhibit the uptake of GABA in the brain, which can decrease anxiety and muscle tension. Cellular synthesis of CBG, via CBGA, derives from olivetolic acid and geranyldiphosphate pathways. Formation of olivetolic acid stems from fatty acid biosynthesis in which hexanoic acid is produced and which in turn is converted to hexanoyl-CoA through hexanoyl CoA synthetase.

Polyketide synthase catalyzes three sequential condensation reactions of malonyl-CoA onto hexanoyl-CoA to form 3,5,7-trioxododecanoyl-CoA which is converted to olivetolic acid (2,4-dihydroxy-6-pentylbenzoate) by the enzyme olivetolic acid cyclase ([OAC] Gagne el al., PNAS, 109: 12811-12816). Formation of geranyldiphosphate stems from the mevalonate pathway (MVA) or methylerythritol-4-phosphate pathway (MEP; also known as the deoxyxylulose-5-phosphate), which produce isopentyl pyrophosphate (IPP) and dimethylallyl pyrophosphate (DMAPP), which are converted to geranyl pyrophosphate (GPP) using geranyl pyrophosphate synthase.

Geranyl-pyrophosphate-olivetolic acid geranyltransferase (EC 2.5.1.102, GOT) catalyzes the following reaction:

geranyl diphosphate+2,4-dihydroxy-6-pentylbenzoate

diphosphate+cannabigerolic acid

The enzyme carrying out the above reaction in C. sativa is a transmembrane prenyltransferase belonging to the UbiA superfamily of membrane proteins. See for example WO2011017798A1 describing CsPTl and WO2018200888 describing CsPT4. However, the above reaction has also been reported to be carried out by a different family of enzymes of bacterial origin. In particular, aromatic prenyltransferases that are soluble, non-transmembrane, and have a 10-stranded antiparallel β-barrel consisting of 5 repeated αββα motifs, can catalyze the transfer of isoprenoid chains to aromatic rings. For example, Kuzuyama T, Noel J P, Richard S B (Nature. 435: 983-987, 2005) and Yang, Y., et al. (Biochemistry 51: 2606-180, 2012) reports that NphB (aka “Orf2”), a Streptomyces-derived, soluble enzyme, catalyzes the attachment of a 10-carbon geranyl group (i.e. GPP) to aromatic substrates; originally identified in the biosynthetic pathway of the antioxidant naphterpin. Yang notes the reaction mechanism of the prenylation step has been characterized as a S(N)l type dissociative mechanism with a weakly stable carbocation intermediate. NphB catalyzes the prenyl transfer between GPP and 1,6 dihydroxynaphthalene (1,6-DHN) and yields three products with the geranyl moiety attaching to different carbon atoms of 1,6-DHN. The major product 5 geranyl DHN and minor product 2-geranyl DHN were characterized with a product ratio of 10:1.

A subsequent publication (Kumano et al. Bioorg. Med. Chem, 16, 8117-8126, 2008), reports rates and regioselectivity measurements for NphB-catalyzed geranylation of olivetol, with mixed regioselectivity at 2- and 4-OL ring positions, and rates of 0.0026 mol 2-geranyl-OL/min/mol NphB and 0.0016 mol 4-geranyl-OL/min/mol NphB, which are extremely slow.

### SUMMARY

Aspects of the disclosure are directed towards forming prenylated aromatic compounds, including cannabinoids, engineered enzymes (e.g., prenyltransferase variants of the soluble aromatic prenyltransferase type) with improved activity that facilitate cannabinoid formation, non-natural cells including the engineered enzymes and prenylated aromatic compound formation, including cannabinoid pathways, fermentation methods using the same, and improved prenylated aromatic compound preparations, including cannabinoid product preparations. In particular, the disclosure is directed towards non-natural prenyltransferases that include at least one amino acid variation that differs from an amino acid residue of a wild type soluble type prenyltransferase.

Unlike CsPT1 and CsPT4 from C. sativa, NphB-type prenyltransferases are water soluble are not multi-transmembrane proteins. As such, these prenyltransferases presented herein are more amenable to heterologous expression in a microbial host, such as bacteria, yeast and microalgae.

In experimental studies associated with the disclosure, prenyltransferase homologs and non-natural prenyltransferase variants were created and identified that demonstrate activity on, or improved activity on catalyzing the reaction between olivetolic acid (OLA) and geranyl diphosphate (GPP) to form the product 3-geranyl-olivetolate (cannabigerolic acid; CBGA, 3-GOLA). Described herein are homologs of NphB and non-natural variants of those prenyltransferase homologs with improved activity and/or regioselectivity.

Novel non-natural prenyltransferase variants were created and identified with improved activities and/or regioselectivity to 3-geranyl-olivetolate, (3-GOLA), forming a predominance of the desired product 3-GOLA (i.e. CBGA) over the less preferred 5-geranyl-olivetolate (5-GOLA). In aspects, the findings of prenyltransferase variants with improved activities and/or regioselectivity to 3-GOLA provide important disclosure as undesired 5-GOLA is the more dominant product in reactions catalyzed by wild-type homologs of NphB. As such, it is preferred to avoid enzyme catalyzed reactions that lead to 5-GOLA when the desired target product is cannabigerolic acid (CBGA). Therefore the disclosure provides the surprising discovery of a significant number of non-natural prenyltransferase variants that have very high regiospecificity towards CBGA (3-GOLA), which can be used for microbial production of prenylated products. Accordingly, these high-activity and regiospecific enzymes can be used according to the current disclosure to catalyze formation of CBGA (3-GOLA) in engineered cells to generate high titers of this molecule which in turn can be used for generating therapeutic and medicinal preparations, including cannabinoids, especially CBGA and its derivatives. Importantly, the CBGA derivatives may include incorporation of different and/or multiple species of hydrophobic substrates (i.e. “donor” molecules).

Non-natural prenyltransferase variants that demonstrated activity on, or improved activity catalyzing the reaction between divarinolic acid (DVA) and geranyl diphosphate (GPP) to form the product cannabigerovarinic acid (CBGVA), as well as those that catalyzed the reaction between orsellinic acid (OSA) and geranyl diphosphate (GPP) to form the product cannabigerorcinic acid (CBGOA), were identified. Non-natural prenyltransferase variants that demonstrated regioselectivity to 3-geranyl-divarinolic acid (3-GDVA), and to 3-geranyl-orsellinate (3-GOSA) were also identified.

In one aspect, a non-natural prenyltransferase is provided comprising at least one amino acid variation as compared to a wild type prenyltransferase, and enzymatically capable of (a) at least two-fold greater rate of formation of 3-geranyl-olivetolate (3-GOLA) from geranyl pyrophosphate and olivetolic acid, as compared to the wild type prenyltransferase; (b) providing regioselectivity to 3-GOLA; or both (a) and (b). In some cases, the non-natural prenyltransferase is enzymatically capable of at least five-fold greater rate of formation of 3-GOLA from geranyl pyrophosphate and olivetolic acid, as compared to the wild type prenyltransferase. In some cases, the non-natural prenyltransferase is enzymatically capable of at least twenty-fold greater rate of formation of 3-GOLA from geranyl pyrophosphate and olivetolic acid, as compared to the wild type prenyltransferase. In some cases, the non-natural prenyltransferase is enzymatically capable of 90% or greater regioselectivity to 3-GOLA. In some cases, the non-natural prenyltransferase is enzymatically capable of 98% or greater regioselectivity to 3-GOLA.

In another aspect, a non-natural prenyltransferase is provided comprising at least one amino acid variation as compared to a wild type prenyltransferase, and enzymatically capable of (a1) at least two fold greater rate of formation of cannabigerovarinic acid (CBGVA) from geranyl pyrophosphate and divarinolic acid (DVA), as compared to the wild type prenyltransferase; (a2) 50% or greater regioselectivity to 3-geranyl-divarinolic acid (3-GDVA); or both (a1) and (a2); or (b1) at least two fold greater rate of formation of cannabigerorcinic acid (CBGOA) from geranyl pyrophosphate and orsellinic acid (OSA), as compared to the wild type prenyltransferase; (b2) 50% or greater regioselectivity to 3-geranyl-orsellinate (3-GOSA); or both (b1) and (b2). In some cases, the non-natural prenyltransferase is enzymatically capable of at least fifty-fold greater rate of formation of: (a) cannabigerovarinic acid (CBGVA) from geranyl pyrophosphate and divarinolic acid (DVA); or (b) cannabigerorcinic acid (CBGOA) from geranyl pyrophosphate and orsellinic acid (OSA), as compared to the wild type prenyltransferase. In some cases, the non-natural prenyltransferase is enzymatically capable of at least two hundred fold greater rate of formation of: (a) cannabigerovarinic acid (CBGVA) from geranyl pyrophosphate and divarinolic acid (DVA); or (b) cannabigerorcinic acid (CBGOA) from geranyl pyrophosphate and orsellinic acid (OSA), as compared to the wild type prenyltransferase. In some cases, the non-natural prenyltransferase is enzymatically capable of 90% or greater regioselectivity to 3-geranyl-divarinolic acid (3-GDVA) or 3-geranyl-orsellinate (3-GOSA). In some cases, the non-natural prenyltransferase is enzymatically capable of 98% or greater regioselectivity to 3-geranyl-divarinolic acid (3-GDVA) or 3-geranyl-orsellinate (3-GOSA).

In another aspect, a non-natural prenyltransferase is provided comprising at least one amino acid variation as compared to a wild type prenyltransferase, and enzymatically capable of regioselectively forming a 2-prenylated 5-alkylbenzene-1,3-diol from geranyl pyrophosphate and 5-alkylbenzene-1,3-diol. In some cases, the 5-alkylbenzene-1,3-diol is olivetol and the 2-prenylated 5-alkylbenzene-1,3-diol is cannabigerol (CBG; 2-GOL). In some cases, the non-natural prenyltransferase is enzymatically capable of 90% or greater regioselectivity to 2-prenylated 5-alkylbenzene-1,3-diol from geranyl pyrophosphate or cannabigerol (CBG; 2-GOL). In some cases, the non-natural prenyltransferase is enzymatically capable of 98% or greater regioselectivity to 2-prenylated 5-alkylbenzene-1,3-diol from geranyl pyrophosphate or cannabigerol (CBG; 2-GOL).

In some cases, the non-natural prenyltransferase comprises at least two amino acid variations as compared to a wild type prenyltransferase. In some cases, the non-natural prenyltransferase comprises at least three, at least four, or at least five amino acid variations as compared to a wild type prenyltransferase. In some cases, the non-natural prenyltransferase has 50% or greater identity to SEQ ID NO: 1 (NphB) or to any one of SEQ ID NOs: 2-27. In some cases, the non-natural prenyltransferase has 90% or greater identity to SEQ ID NO: 1 (NphB) or to any one of SEQ ID NOs: 2-27. In some cases, the at least one amino acid variation is made to the wild type prenyltransferase SEQ ID NO: 1 (NphB) or in any one of SEQ ID NOs: 2-27. In some cases, the non-natural prenyltransferase comprises one or more amino acid variations at position(s) selected from the group consisting of: 17, 25, 38, 49, 51, 53, 106, 108, 112, 118, 119, 121, 123, 126, 161, 162, 166, 173, 174, 177, 205, 209, 213, 214, 216, 219, 227, 228, 230, 232, 234, 269, 270, 271, 274, 283, 286, 287, 288, 294, 295, 298, and 302, relative to SEQ ID NO: 1 (NphB). In some cases, the non-natural prenyltransferase comprises one or more amino acid variations at position(s) selected from the group consisting of: A17T, C25V, Q38G, V49A, V49L, V49S, S51T, A53C, A53D, A53E, A53F, A53G, A53H, A53I, A53K, A53L, A53M, A53N, A53P, A53Q, A53R, A53S, A53T, A53V, A53W, A53Y, M106E, A108G, E112D, E112G, K118N, K118Q, K119A, K119D, Y121W, F123L, F123A, F123H, F123W, T126R, Q161H, Q161R, Q161S, Q161T, Q161Y, Q161A, Q161F, Q161G, Q161I, Q161K, Q161L, Q161M, Q161C, Q161D, Q161E, Q161N, Q161P, Q161V, Q161W, M162A, M162F, D166E, N173D, L174V, S177E, S177W, S177Y, S177H, S177K, S177R, G205L, G205M, C209G, F213M, S214A, S214C, S214D, S214E, S214F, S214G, S214I, S214K, S214L, S214M, S214N, S214P, S214Q, S214R, S214T, S214V, S214W, S214Y, S214H, Y216A, L219F, D227E, R228E, R228Q, C230N, C230S, A232S, I234H, T269W, L270Y, V271E, L274V, Y283L, G286E, A287Y, Y288A, Y288F, Y288L, Y288M, Y288P, Y288T, Y288V, Y288C, Y288D, Y288E, Y288G, Y288H, Y288I, Y288K, Y288N, Y288Q, Y288R, Y288S, Y288W, V294A, V294F, V294N, Q295G, Q295K, Q295L, Q295N, Q295P, Q295R, Q295F, Q295W, Q295H, Q295C, Q295A, Q295S, Q295V, Q295D, Q295Y, Q295E, Q295I, Q295M, Q295T, L298A, L298Q, L298W, and F302K. In some cases, the non-natural prenyltransferase comprises one or more amino acid variations at position(s) selected from the group consisting of: a) S214H; b) Y288V; c) Q161H; d) Q161R and Q295V; e) Q161S and Q295F; f) Q161S and Q295L; g) Q161S and S177W; h) Q161S and S214R; i) Q161H and Q295V; j) Q161H and Q295W; k) S214R and Q295F; 1) S214R and Q295F; m) V49A and Q295L; n) V49A and S214R; o) Y288I and Q295V; p) S177W and Q295A; q) S177W and S214R; r) A53T and Q161S; s) A53T and Q295A; t) A53T and Q295F; u) A53T and Q295W; v) A53T and S177W; w) A53T and 5214R; x) A53T and V294A; y) Q161S, V294A, and Q295A; z) Q161S, V294A, and Q295W; aa) Q161H, Y288I, and Q295W; bb) Q161H, Y288V, and Q295M; cc) A53T, Q161S, and Q295A; dd) A53T, Q161S, and Q295W; ee) A53T, Q161S, and V294A; ff) A53T, Q161S, and V294N; gg) A53T, V294A, and Q295A; hh) A53T, V294A, and Q295W; ii) Q161 S, S214H, and Y288V; jj) A53T, Q161S, V294A, and Q295A; kk) A53T, Q161S, V294A, and Q295W; 11) A53T, Q161S, V294N, and Q295A; and mm) A53T, Q161S, V294N, and Q295W. In some cases, the non-natural prenyltransferase has identity to SEQ ID NO: 1 (NphB) in the range of 35% to 95%. In some cases, the non-natural prenyltransferase comprises one or more amino acid variations at position(s) selected from the group consisting of: 17, 25, 38, 47, 49, 51, 104, 106, 110, 116, 117, 119, 121, 124, 159, 160, 164, 171, 172, 175, 203, 207, 211, 212, 214, 217, 225, 226, 228, 230, 232, 267, 268, 269, 272, 281, 284, 285, 286, 292, 293, 296, and 300, relative to SEQ ID NO: 16.

In some cases, the non-natural prenyltransferase comprises one or more amino acid variations at position(s) selected from the group consisting of: A17T, C25V, Q38G, V47A, V47L, V47S, S49T, A51C, A51D, A51E, A51F, A51G, A51H, A51I, A51K, A51L, A51M, A51N, A51P, A51Q, A51R, A51S, A51T, A51V, A51W, A51Y, M104E, A106G, E110D, E110G, K116N, K116Q, K117A, K117D, Y119W, F121L, F121A, F121H, F121W, T124R, Q159H, Q159R, Q159S, Q159T, Q159Y, Q159A, Q159F, Q159G, Q159I, Q159K, Q159L, Q159M, Q159C, Q159D, Q159E, Q159N, Q159P, Q159V, Q159W, M160A, M160F, D164E, N171D, L172V, S175E, S175W, S175Y, S175H, S175K, S175R, G203L, G203M, C207G, F211M, S212A, S212C, S212D, S212E, S212F, S212G, S212I, S212K, S212L, S212M, S212N, S212P, S212Q, S212R, S212T, S212V, S212W, S212Y, S212H, Y214A, L217F, D225E, R226E, R226Q, C228N, C228S, A230S, I232H, T267W, L268Y, V269E, L272V, Y281L, G284E, A285Y, Y286A, Y286F, Y286L, Y286M, Y286P, Y286T, Y286V, Y286C, Y286D, Y286E, Y286G, Y286H, Y286I, Y286K, Y286N, Y286Q, Y286R, Y286S, Y286W, I292A, I292F, I292N, Q293G, Q293K, Q293L, Q293N, Q293P, Q293R, Q293F, Q293W, Q293H, Q293C, Q293A, Q293S, Q293V, Q293D, Q293Y, Q293E, Q293I, Q293M, Q293T, L296A, L296Q, L296W, and F300K, relative to SEQ ID NO: 16.

In some cases, the non-natural prenyltransferase comprises at least two amino acid variations at positions selected from: (i) Q159A, and (ii) Q293F, Q293M, Q293F, or Q293F; (i) Q159F, and (ii) Q293F, Q293W, or Q293H; (i) Q159G, and (ii) Q293F; (i) Q159H, and (ii) Q293W, Q293H, Q293C, Q293A, Q293S, Q293V, Q293D, Q293Y, or Q293E; (i) Q159I, and (ii) Q293F; (i) Q159K, and (ii) Q293V or Q293V; (i) Q159L, and (ii) Q293W or Q293F; (i) Q159M, and (ii) Q293F or Q293W; (i) Q159R, and (ii) Q293V, Q293M, or Q293T; (i) Q159S, and (ii) Y286I; (i) S175H, and (ii) Q293V; (i) A51T, and (ii) Q293A; (i) A51T, and (ii) Q293W; (i) A51T, and (ii) I292A; (i) S175W, and (ii) Q293A; (i) A51T, and (ii) S175W; (i) A51T, and (ii) Q293F; (i) A51T, and (ii) 5212R; (i) A51T, and (ii) Q159S; (i) Q159S and (ii) Q293F; (i) Q159S and (ii) Q293L; (i) S212R and (ii) Q293F; (i) Q159S and (ii) S212R; (i) S175W and (ii) S212R; (i) V47A and (ii) S212R; (i) Q159S and (ii) S175W; and (i) V47A and (ii) Q293, relative to SEQ ID NO: 16.

In some cases, the non-natural prenyltransferase comprises at least three amino acid variations at positions selected from: (i) Q159H, (ii) Y286A, and (iii) Q293F, Q293M, or Q293V; (i) Q159H, (ii) Y286I, and (iii) Q293M or Q293V; (i) Q159H, (ii) Y286V, and (iii) Q293F, Q293M, Q293V, or Q293W; (i) Q159L, (ii) S175H, and (iii) Q293F; (i) S175H, (ii), Y286V, and (iii) Q293M; (i) S175H, (ii), Y286I, and (iii) Q293M or Q293V; (i) Q159S, (ii) S175H, and (iii) Y286I; (i) Q159S, (ii) S175R, and (iii) Y286V; (i) Q159S, (ii) S175S, and (iii) Y286I; (i) Q159S, (ii) S212H, and (iii) Y286A or Y286V; (i) Q159S, (ii) I292A, and (iii) Q293W; (i) A51T, (ii) Q159S, and (iii) Q293W; (i) A51T, (ii) I292A, and (iii) Q293A; (i) A51T, (ii) I292A, and (iii) Q293W; (i) A51T, (ii) Q159S, and (iii) Q293A; (i) A51T, (ii) Q159S, and (iii) I292A; (i) A51T, (ii) Q159S, and (iii) I292N; and (i) Q159S, (ii) I292A, and (iii) Q293A, relative to SEQ ID NO: 16.

In some cases, the non-natural prenyltransferase comprises at least four amino acid variations at positions selected from: (i) Q159H, (ii) S175H, (iii) Y286A, and (iv) Q293V; (i) Q159H, (ii) S175H, (iii) Y286V, and (iv) Q293M or Q293V; (i) Q159H, (ii) S175R, (iii) Y286I, and (iv) Q293M; (i) Q159L, (ii) S175K, (iii) Y286A, and (iv) Q293V; (i) Q159M, (ii) S175H, (iii) Y286V, and (iv) Q293F; (i) Q159R, (ii) S175H, (iii) Y286I, and (iv) Q293Q; (i) Q159S, (ii) S175H, (iii) Y286V, and (iv) Q293F; (i) Q159S, (ii) S175K, (iii) Y286V, and (iv) Q293V; (i) Q159S, (ii) S212H, (iii) Y286V, and (iv) Q293M; (i) A51T, (ii) Q159S, (iii) I292A, and (iv) Q293W; (i) A51T, (ii) Q159S, (iii) I292N, and (iv) Q293W; (i) A51T, (ii) Q159S, (iii) I292A, and (iv) Q293A; (i) A51T, (ii) Q159S, (iii) I292N, and (iv) Q293A; and (i) A51T, (ii) Q159S, (iii) I292N, and (iv) Q293A, relative to SEQ ID NO: 16.

In some cases, the non-natural prenyltransferase comprises at least five amino acid variations at positions selected from: (i) Q159H, (ii) S175R, (iii) S212H, (iv) Y286A, and (v) Q293V; and (i) Q159R, (ii) S175R, (iii) S212H, (iv) Y286I, and (v) Q293M, relative to SEQ ID NO: 16.

In some cases, the non-natural prenyltransferase further comprises one or more amino acid variations at positions selected from: (i) F211N, F211S, A230S, G284S, and Y286N, relative to SEQ ID NO 16; or (ii) F213N, F213S, A232S, G286S, and Y288N, relative to SEQ ID NO: 1 (NphB).

In yet another aspect, a nucleic acid is provided encoding a non-natural prenyltransferases of any of the preceding. In yet another aspect, an expression construct is provided comprising the nucleic acid.

In another aspect, an engineered cell is provided comprising a non-natural prenyltransferase comprising at least one amino acid variation as compared to a wild type prenyltransferase, and enzymatically capable of (a) at least two fold greater rate of formation of cannabigerolic acid (CBGA) from geranyl pyrophosphate and olivetolic acid, as compared to the wild type prenyltransferase; (b) 50% or greater regioselectivity to 3-geranyl-olivetolate (3-GOLA); or both (a) and (b).

In another aspect, an engineered cell is provided comprising a non-natural prenyltransferase comprising at least one amino acid variation as compared to a wild type prenyltransferase, and enzymatically capable of: a1) at least two fold greater rate of formation of cannabigerovarinic acid (CBGVA) from geranyl pyrophosphate and divarinolic acid (DVA), as compared to the wild type prenyltransferase; (a2) 50% or greater regioselectivity to 3-geranyl-divarinolic acid (3-GDVA); or both (a1) and (a2); or (b1) at least two fold greater rate of formation of cannabigerorcinic acid (CBGOA) from geranyl pyrophosphate and orsellinic acid (OS A), as compared to the wild type prenyltransferase; (b2) 50% or greater regioselectivity to 3-geranyl-orsellinate (3-GOSA); or both (b1) and (b2).

In another aspect, an engineered cell is provided comprising a non-natural prenyltransferase comprising at least one amino acid variation as compared to a wild type prenyltransferase, and enzymatically capable of regioselectively forming a 2-prenylated 5-alkylbenzene-1,3-diol from geranyl pyrophosphate and 5-alkylbenzene-1,3-diol. In some cases, the 5-alkylbenzene-1,3-diol is olivetol and the prenylated alcohol 2-prenylated 5-alkylbenzene-1,3-diol is cannabigerol (CBG; 2-GOL). In some cases, the engineered cell comprises a non-natural prenyltransferase of any of the preceding or a nucleic acid of any of the preceding. In some cases, the engineered cell comprises an olivetolic acid pathway. In some cases, the olivetolic acid pathway comprises polyketide synthase/olivetol synthase (condensation of hexanoyl coenzyme A (CoA) and malonyl CoA) along with olivetolic acid cyclase (OAC). In some cases, the engineered cell comprises a DVA or OSA pathway. In some cases, the engineered cell comprises an olivetol pathway. In some cases, the olivetol pathway comprises polyketide synthase. In some cases, the engineered cell comprises a geranyl pyrophosphate pathway. In some cases, the geranyl pyrophosphate (GPP) pathway comprises geranyl pyrophosphate synthase. In some cases, the GPP pathway comprises a mevalonate (MVA) pathway, a MEP pathway, or both. In some cases, the engineered cell comprises two or more exogenous nucleic acids, wherein one of the two or more exogenous nucleic acids encodes the non-natural prenyltransferase. In some cases, the exogenous nucleic acids encode an enzyme in (a) the olivetolic acid pathway, (b) the geranyl pyrophosphate pathway, or both (a) and (b). In some cases, the exogenous nucleic acids encode an enzyme in (a) the DVA or OSA pathway, (b) the geranyl pyrophosphate pathway, or both (a) and (b). In some cases, the exogenous nucleic acids encode an enzyme in (a) the olivetol pathway, (b) the geranyl pyrophosphate pathway, or both (a) and (b). In some cases, the engineered cell is selected from the group consisting of yeast, microalgae, Escherichia, Corynebacterium, Bacillus, Ralstonia, and Staphylococcus.

In another aspect, a cell extract or cell culture medium is provided comprising cannabigerolic acid (CBGA) derived from the engineered cell of any of the preceding. In some cases, the cell extract or cell culture medium comprises cannabigerolic acid (CBGA) at 50% or greater of the total geranyl olivetolate (3-GOLA plus 5-GOLA) or comprising CBG at 50% or greater of the total CBG (2-GOL) plus 4-GOL.

In yet another aspect, a purified cannabigerolic acid (CBGA) or CBG is provided derived from the engineered cell of any of the preceding, or the cell extract or cell culture medium of any of the preceding. In some cases, the purified cannabigerolic acid (CBGA) or CBG comprises cannabigerolic acid (CBGA) at 50% or greater of the total geranyl olivetolate (3-GOLA plus 5-GOLA) or comprising CBG at 50% or greater of the total CBG (2-GOL) plus 4-GOL.

In yet another aspect, a cell extract or cell culture medium is provided comprising CBGVA or CBGOA derived from the engineered cell of any of the preceding.

In yet another aspect, a purified CBGVA or CBGOA is provided derived from the engineered cell of any of the preceding, or the cell extract or cell culture medium of any of the preceding.

In yet another aspect, a cell extract or cell culture medium is provided comprising cannabigerol (CBG) derived from the engineered cell of any of the preceding.

In yet another aspect, a purified cannabigerol (CBG) is provided derived from the engineered cell of any of the preceding, or the cell extract or cell culture medium of any of the preceding.

In another aspect, a method is provided for forming a prenylated aromatic compound, comprising contacting a hydrophobic substrate and an aromatic substrate with a non-natural prenyltransferase of any of the preceding, wherein contacting forms a prenylated aromatic compound. In some cases, the aromatic substrate is selected from the group consisting of olivetol, olivetolic acid, divarinol, divarinolic acid, orcinol, and orsellinic acid. In some cases, the hydrophobic substrate includes any one of an isoprenoid portion, a geranyl portion, a farnesyl portion, and one or more phosphate groups. In some cases, the contacting occurs in the engineered cell of any of the preceding. In some cases, the method further comprises isolating or purifying the prenylated aromatic compound, or a derivative thereof, from other material. In some cases, the isolating or purifying comprises one or more of continuous liquid-liquid extraction, pervaporation, evaporation, filtration, membrane filtration (including reverse osmosis, nanofiltration, ultrafiltration, and microfiltration), membrane filtration with diafiltration, membrane separation, reverse osmosis, electrodialysis, distillation, extractive distillation, reactive distillation, azeotropic distillation, crystallization and recrystallization, centrifugation, extractive filtration, ion exchange chromatography, size exclusion chromatography, adsorption chromatography, carbon adsorption, hydrogenation, and ultrafiltration.

In another aspect, a method of making a therapeutic composition including geranyl olivetolate, or a derivative thereof is provided, the method comprising including the geranyl olivetolate, or a derivative thereof, obtained from the engineered cell of any of the preceding, or the method of any of the preceding, in a therapeutic composition.

In another aspect, a therapeutic or a medicinal composition including cannabigerolic acid (CBGA), or a derivative thereof is provided, obtained from the engineered cell of any of the preceding, or the method of any of the preceding. In some cases, the derivative thereof is CBG. In some cases, the therapeutic or medicinal composition comprises CBGA or CBG at 60% or greater, 70% or greater, 80% or greater, 85% or greater, 90% or greater, 91% or greater, 92% or greater, 93% or greater, 94% or greater, 95% or greater, 96% or greater, 97% or greater, 98% or greater, 99% or greater, 99.2% or greater, 99.4% or greater, 99.5% or greater, 99.6% or greater, 99.7% or greater, 99.8% or greater, or 99.9% or greater of total cannabinoid compound(s) in the therapeutic composition.

In another aspect, a method of making a therapeutic composition including CBGVA or CBGOA, or a derivative thereof is provided, the method comprising including the CBGVA or CBGOA, or a derivative thereof, obtained from the engineered cell of any of the preceding, or the method of any of the preceding, in a therapeutic composition.

In another aspect, a therapeutic or a medicinal composition including CBGVA or CBGOA, or a derivative thereof is provided, obtained from the engineered cell of any of the preceding, or the method of any of the preceding. In some cases, the therapeutic or medicinal composition comprises CBGVA or CBGOA at 60% or greater, 70% or greater, 80% or greater, 85% or greater, 90% or greater, 91% or greater, 92% or greater, 93% or greater, 94% or greater, 95% or greater, 96% or greater, 97% or greater, 98% or greater, 99% or greater, 99.2% or greater, 99.4% or greater, 99.5% or greater, 99.6% or greater, 99.7% or greater, 99.8% or greater, or 99.9% or greater of total cannabinoid compound(s) in the therapeutic composition.

In another aspect, a method of making a therapeutic composition including CBG is provided, comprising including the CBG obtained from the engineered cell of any of the preceding, or the method of any of the preceding, in a therapeutic composition.

In another aspect, a therapeutic or a medicinal composition including CBG is provided obtained from the engineered cell of any of the preceding, or the method of any of the preceding. In some cases, the therapeutic or medicinal composition comprises CBG at 60% or greater, 70% or greater, 80% or greater, 85% or greater, 90% or greater, 91% or greater, 92% or greater, 93% or greater, 94% or greater, 95% or greater, 96% or greater, 97% or greater, 98% or greater, 99% or greater, 99.2% or greater, 99.4% or greater, 99.5% or greater, 99.6% or greater, 99.7% or greater, 99.8% or greater, or 99.9% or greater of total cannabinoid compound(s) in the therapeutic composition.

In some aspects, the current disclosure provides non-natural prenyltransferases that include at least one amino acid variation as compared to a wild type prenyltransferase. Non-natural prenyltransferases of the disclosure include those that are (a) enzymatically capable of at least two fold greater rate of formation of geranyl-olivetolate from geranyl pyrophosphate and olivetolic acid, as compared to the wild type prenyltransferase or; (b) regioselective to 3-geranyl-olivetolate (3-GOLA); or both (a) and (b).

In some aspects, the current disclosure also provides non-natural prenyltransferases that include at least one amino acid variation as compared to a wild type prenyltransferase that are enzymatically capable of: (a1) at least two fold greater rate of formation of cannabigerovarinic acid (CBGVA) from geranyl pyrophosphate and divarinolic acid (DVA), as compared to the wild type prenyltransferase; (a2) 50% or greater regioselectivity to 3-geranyl-divarinolic acid (3-GDVA), or both (a1) and (a2); or (b1) at least two fold greater rate of formation of cannabigerorcinic acid (CBGOA) from geranyl pyrophosphate and orsellinic acid (OSA), as compared to the wild type prenyltransferase; (b2) 50% or greater regioselectivity to 3-geranyl-orsellinate (3-GOSA); or both (b1) and (b2).

In some aspects, the disclosure also provides a non-natural prenyltransferase comprising at least one amino acid variation as compared to a wild type prenyltransferase, and enzymatically capable of regioselectively forming a 2-prenylated 5-alkylbenzene-1,3-diol from geranyl pyrophosphate and 5-alkylbenzene-1,3-diol. For example, the 5-alkylbenzene-1,3-diol substrate can be olivetol and the 2-prenylated 5-alkylbenzene-1,3-diol can be cannabigerol (CBG; 2-GOL).

The variant prenyltransferases of the disclosure have at least one amino acid substitution as compared to its corresponding natural prenyltransferases of the soluble, αββα (ABBA) structural type, or a prenyltransferase having one or more variations that are different than one or more variations that provide improved activity and/or regioselectivity to 3-GOLA. For example, a prenyltransferase with a different mutation which may have been previously engineered can be used as a template, prior to incorporating any modification described herein. Such prenyltransferases that are starting sequences for incorporating a modification described herein to generate the novel engineered enzyme may be alternatively referred to herein as wild-type, template, starting sequence, natural, naturally-occurring, unmodified, corresponding natural prenyltransferases, corresponding natural prenyltransferases without the amino acid substitution, corresponding prenyltransferases or corresponding prenyltransferases without the amino acid substitution(s). Experimental studies described demonstrate that a number of amino acid positions along the length of the prenyltransferase sequence can be substituted to provide non-natural prenyltransferases having increased activity and desired regioselectivity. Experimental studies associated with the disclosure show single substitutions and combinations of substitutions in a prenyltransferase template can provide increased activity and desired regioselectivity, and therefore provide single and combination variants of a starting or template or corresponding prenyltransferases, e.g., in particular enzymes of the class EC 2.5.1.102, having increased substrate conversion and/or regioselectivity.

This disclosure provides a set of 27 natural homologs that may serve as templates for introducing orthologous substitutions to improve substrate conversion and/or regioselectivity, which in turn may enhance capabilities to produce a diversity of prenylation aromatic molecules.

Amino acid variations can include those relative to SEQ ID NO: 1 (NphB) or a homolog thereof, having one or more variations at position(s) selected from the group consisting of: A17T, C25V, Q38G, V49A, V49L, V49S, S51T, A53C, A53D, A53E, A53F, A53G, A53H, A53I, A53K, A53L, A53M, A53N, A53P, A53Q, A53R, A53S, A53T, A53V, A53W, A53Y, M106E, A108G, E112D, E112G, K118N, K118Q, K119A, K119D, Y121W, F123L, F123A, F123H, F123W, T126R, Q161H, Q161R, Q161S, Q161T, Q161Y, Q161A, Q161F, Q161G, Q161I, Q161K, Q161L, Q161M, Q161C, Q161D, Q161E, Q161N, Q161P, Q161V, Q161W, M162A, M162F, D166E, N173D, L174V, S177E, S177W, S177Y, S177H, S177K, S177R, G205L, G205M, C209G, F213M, S214A, S214C, S214D, S214E, S214F, S214G, S214I, S214K, S214L, S214M, S214N, S214P, S214Q, S214R, S214T, S214V, S214W, S214Y, S214H, Y216A, L219F, D227E, R228E, R228Q, C230N, C230S, A232S, I234H, T269W, L270Y, V271E, L274V, Y283L, G286E, A287Y, Y288A, Y288F, Y288L, Y288M, Y288P, Y288T, Y288V, Y288C, Y288D, Y288E, Y288G, Y288H, Y288I, Y288K, Y288N, Y288Q, Y288R, Y288S, Y288W, V294A, V294F, V294N, Q295G, Q295K, Q295L, Q295N, Q295P, Q295R, Q295F, Q295W, Q295H, Q295C, Q295A, Q295S, Q295V, Q295D, Q295Y, Q295E, Q295I, Q295M, Q295T, L298A, L298Q, L298W, and F302K.

Amino acid variations can include those relative to SEQ ID NO: 16 or a homolog thereof, having one or more variations at position(s) selected from the group consisting of: A17T, C25V, Q38G, V47A, V47L, V47S, S49T, A51C, A51D, A51E, A51F, A51G, A51H, A51I, A51K, A51L, A51M, A51N, A51P, A51Q, A51R, A51S, A51T, A51V, A51W, A51Y, M104E, A106G, E110D, E110G, K116N, K116Q, K117A, K117D, Y119W, F121L, F121A, F121H, F121W, T124R, Q159H, Q159R, Q159S, Q159T, Q159Y, Q159A, Q159F, Q159G, Q159I, Q159K, Q159L, Q159M, Q159C, Q159D, Q159E, Q159N, Q159P, Q159V, Q159W, M160A, M160F, D164E, N171D, L172V, S175E, S175W, S175Y, S175H, S175K, S175R, G203L, G203M, C207G, F211M, S212A, S212C, S212D, S212E, S212F, S212G, S212I, S212K, S212L, S212M, S212N, S212P, S212Q, S212R, S212T, S212V, S212W, S212Y, S212H, Y214A, L217F, D225E, R226E, R226Q, C228N, C228S, A230S, I232H, T267W, L268Y, V269E, L272V, Y281L, G284E, A285Y, Y286A, Y286F, Y286L, Y286M, Y286P, Y286T, Y286V, Y286C, Y286D, Y286E, Y286G, Y286H, Y286I, Y286K, Y286N, Y286Q, Y286R, Y286S, Y286W, I292A, I292F, I292N, Q293G, Q293K, Q293L, Q293N, Q293P, Q293R, Q293F, Q293W, Q293H, Q293C, Q293A, Q293S, Q293V, Q293D, Q293Y, Q293E, Q293I, Q293M, Q293T, L296A, L296Q, L296W, and F300K.

Information based on crystal structure of NphB (PDB #1ZB6_A), allowed prediction of specific amino acid positions and identities that provided improved activity and/or regioselectivity, and then by using sequence alignment and 3-D modeling information of various other prenyltransferase homologs, the orthologous amino acid positions were identified that were suspected to also improve activity and/or regioselectivity for the respective homologs, assuming that basal enzymatic activity was present. Accordingly, the disclosure provides non-natural prenyltransferases with regards to orthologous amino acid positions and amino acid variants therein, wherein a non-natural prenyltransferase having 35% or greater identity to any one of SEQ ID NOs: 2-27 and having one or more substitutions that are orthologous to the following positions in SEQ ID NO: 1 (NphB): A17T, C25V, Q38G, V49A, V49L, V49S, S51T, A53C, A53D, A53E, A53F, A53G, A53H, A53I, A53K, A53L, A53M, A53N, A53P, A53Q, A53R, A53S, A53T, A53V, A53W, A53Y, M106E, A108G, E112D, E112G, K118N, K118Q, K119A, K119D, Y121W, F123L, F123A, F123H, F123W, T126R, Q161H, Q161R, Q161S, Q161T, Q161Y, Q161A, Q161F, Q161G, Q161I, Q161K, Q161L, Q161M, Q161C, Q161D, Q161E, Q161N, Q161P, Q161V, Q161W, M162A, M162F, D166E, N173D, L174V, S177E, S177W, S177Y, S177H, S177K, S177R, G205L, G205M, C209G, F213M, S214A, S214C, S214D, S214E, S214F, S214G, S214I, S214K, S214L, S214M, S214N, S214P, S214Q, S214R, S214T, S214V, S214W, S214Y, S214H, Y216A, L219F, D227E, R228E, R228Q, C230N, C230S, A232S, I234H, T269W, L270Y, V271E, L274V, Y283L, G286E, A287Y, Y288A, Y288F, Y288L, Y288M, Y288P, Y288T, Y288V, Y288C, Y288D, Y288E, Y288G, Y288H, Y288I, Y288K, Y288N, Y288Q, Y288R, Y288S, Y288W, V294A, V294F, V294N, Q295G, Q295K, Q295L, Q295N, Q295P, Q295R, Q295F, Q295W, Q295H, Q295C, Q295A, Q295S, Q295V, Q295D, Q295Y, Q295E, Q295I, Q295M, Q295T, L298A, L298Q, L298W, and F302K. (Orthologous sites may be inferred from the multiple sequence alignment in FIG. 5, and respective amino acid positions for each homologous prenyltransferase may be inferred from the submitted sequences SEQ ID NOs: 1-27.)

Some aspects of the current disclosure are directed to an engineered cell expressing a non-natural prenyltransferase comprising at least one amino acid substitution (including single and combination variants). The cells can be used to promote production of a cannabinoid, CBGA (3-GOLA), or a derivative thereof. Aspects of the engineered cell may further optionally include one or more additional metabolic pathway transgene(s) to promote improved cannabinoid formation by increasing cannabinoid precursor flux, to generate a cannabinoid derivative, or to improve recovery of the cannabinoid from the engineered cell.

Other aspects are directed to compositions including an engineered cell, such as cell culture compositions, and also compositions including one or more product(s) produced from the engineered cell. For example, a composition can include a target cannabinoid product produced by the cells, where the composition has been purified to remove cells or other components useful for cell culturing. The composition may be treated to enrich or purify the target product or intermediate thereof.

Other aspects are directed to methods for forming a prenylated aromatic compound. The method may include a step of contacting a hydrophobic substrate and an aromatic substrate with a non-natural prenyltransferase of the disclosure, wherein contacting forms a prenylated aromatic compound. Exemplary aromatic substrates include olivetol, olivetolic acid, divarinol, divarinolic acid, orcinol, and orsellinic acid. The hydrophobic substrate can include an isoprenoid portion, such as geranyl or farnesyl portions or dimethylallyl portions, and can include phosphate groups.

Other aspects of the disclosure are directed to products made from the target cannabinoid product obtained from methods using the engineered cell. Exemplary products include therapeutic or pharmaceutical compositions, medicinal compositions, systems for in vitro use, diagnostic compositions, and precursor compositions for further chemical modification (e.g., decarboxylation of CBGA to CBG by for example heat or a biocatalyst).

Other aspects of the disclosure are directed to nucleic acids encoding the non-natural prenyltransferases with one or more variant amino acids, as well as expression constructs including the nucleic acids, and engineered cells comprising the nucleic acids or expression constructs.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows prenyltransferase-catalyzed reaction of olivetolic acid (OLA) and geranyl diphosphate (GPP) to form the products 3-geranyl-olivetolate (3-GOLA; cannabigerolic acid; CBGA) and 5-geranyl-olivetolate (5-GOLA).

FIG. 2 is a diagram of exemplary metabolic pathways showing 3-GOLA formation from hexanoyl-CoA and geranyl diphosphate.

FIG. 3 shows the chemical structures of various aromatic substrate molecules that can be used in a prenyltransferase catalyzed reaction as substrates.

FIG. 4A is a phylogenetic tree (unrooted Neighbor Joining tree) representing the relatedness between the prenyltransferase homologs (SEQ ID NOs: 1-27)

FIG. 4B is the similarity matrix (measured in percent identity) for all pairwise comparisons of the prenyltransferase homologs (SEQ ID NOs: 1-27).

FIG. 5 shows a multiple sequence alignment (MSA) of NphB (SEQ ID NO: 1) to other prenyltransferase homologs (SEQ ID NOs: 2-27).

FIGS. 6A-6C are tables describing prenyltransferase amino acid positions and variant residues for NphB that were identified via HT screening of prenyltransferase variant libraries and that affect activity, selectivity, or both activity and selectivity on OLA, DVA, and OL, respectively with GPP donor.

FIG. 7A shows reaction of DVA with GPP to form CBGVA.

FIG. 7B shows reaction of OSA with GPP to form CBGOA.

FIG. 8 shows reaction of olivetol with GPP to form CBG.

FIG. 9A shows reaction of DVA with DMAPP to form the respective prenylation product.

FIG. 9B shows reaction of OSA with DMAPP to form the respective prenylation product.

FIG. 10 shows reaction of divarinol with DMAPP to form the respective prenylation product.

FIG. 11 shows crystal structure model for prenyltransferases SEQ ID NO: 1, SEQ ID NO: 15 and SEQ ID NO: 16 at positions orthologous to sites S214, Q161 and Q295 on SEQ ID NO: 1.

FIG. 12A and FIG. 12B show predicted mechanism to model the effect of select amino acid substitutions at sites S214, Q161 and Q295 on SEQ ID NO: 1.

FIG. 13 shows enzyme activity data for NphB for site Q161 for all non-natural prenyltransferase variants from a site saturation amino acid substitution series.

FIG. 14 shows enzyme activity data for NphB for site S214 for all non-natural prenyltransferase variants from a site saturation amino acid substitution series.

FIG. 15 shows enzyme activity data for NphB for site Q295 for all non-natural prenyltransferase variants from a site saturation amino acid substitution series.

FIG. 16 shows enzyme activity data for non-natural prenyltransferase variants derived from SEQ ID NO: 16 and SEQ ID NO: 23 scaffolds.

### DETAILED DESCRIPTION

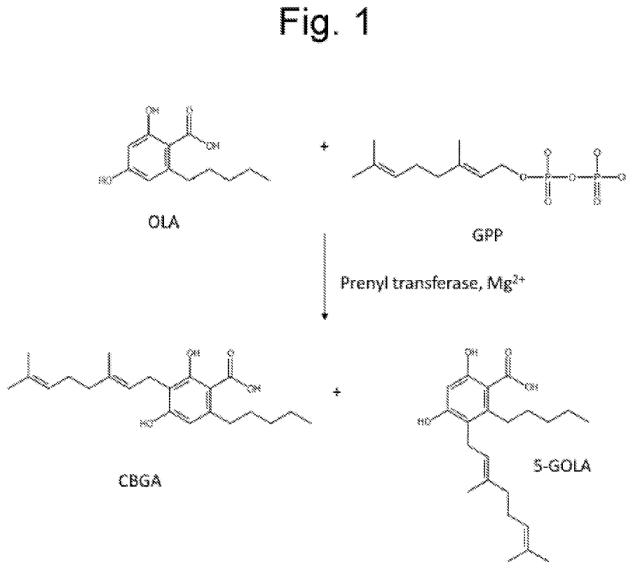

Generally, the disclosure provides non-natural prenyltransferases that are (a) enzymatically capable of at least two fold greater rate of formation of 3-geranyl-olivetolate (3-GOLA; cannabigerolic acid; CBGA) from geranyl pyrophosphate and olivetolic acid, as compared to the wild type prenyltransferase or; (b) regioselective to CBGA (3-geranyl-olivetolate, 3-GOLA); or both (a) and (b). Nucleic acids encoding the non-natural prenyltransferases, as well as expression constructs including the nucleic acids, and engineered cells comprising the nucleic acids or expression constructs are described. FIG. 1 shows reaction of olivetolic acid (OLA) and geranyl diphosphate (GPP) to form the products 3-geranyl-olivetolate (3-GOLA; cannabigerolic acid; CBGA) and 5-geranyl-olivetolate (5-GOLA).

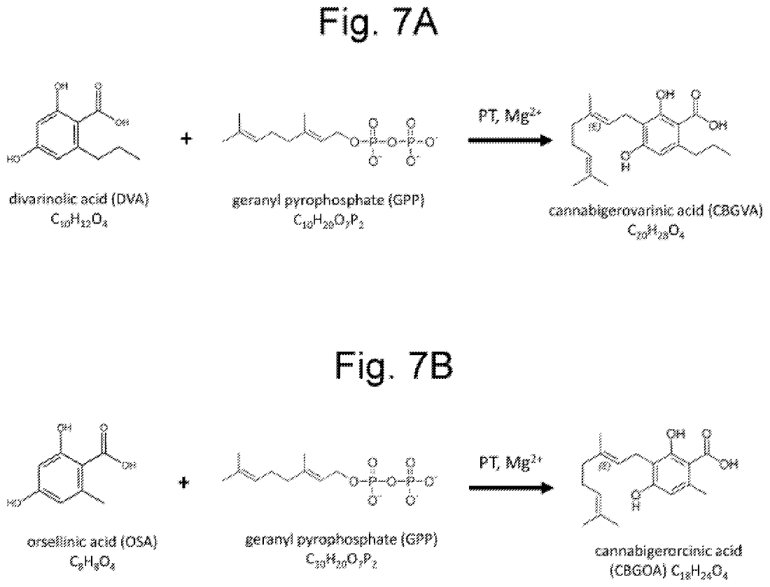

The disclosure also provides non-natural prenyltransferases that include at least one amino acid variation enzymatically capable of either (a1) at least two fold greater rate of formation of cannabigerovarinic acid (CBGVA) from geranyl pyrophosphate and divarinolic acid (DVA), as compared to the wild type prenyltransferase; (a2) 50% or greater regioselectivity to 3-geranyl-divarinolic acid (3-GDVA), or both (a1) and (a2); or (b1) at least two fold greater rate of formation of cannabigerorcinic acid (CBGOA) from geranyl pyrophosphate and orsellinic acid (OSA), as compared to the wild type prenyltransferase; (b2) 50% or greater regioselectivity to 3-geranyl-orsellinate (3-GOSA); or both (b1) and (b2). FIG. 7A shows reaction of divarinolic acid (DVA) and geranyl diphosphate (GPP) to form the product cannabigerovarinic acid (CBGVA); and FIG. 7B shows reaction of orsellinic acid (OSA) and geranyl diphosphate (GPP) to form the product cannabigerorcinic acid (CBGOA).

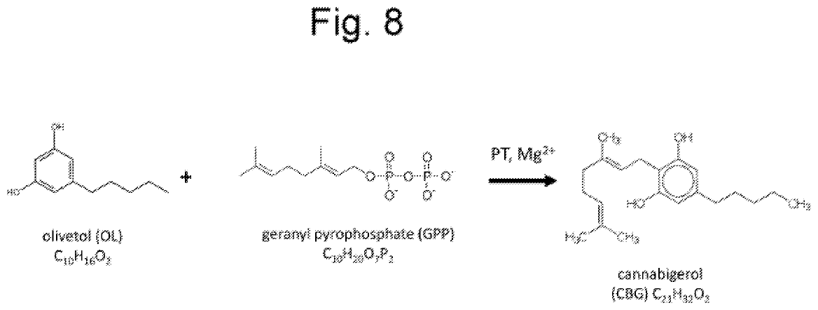

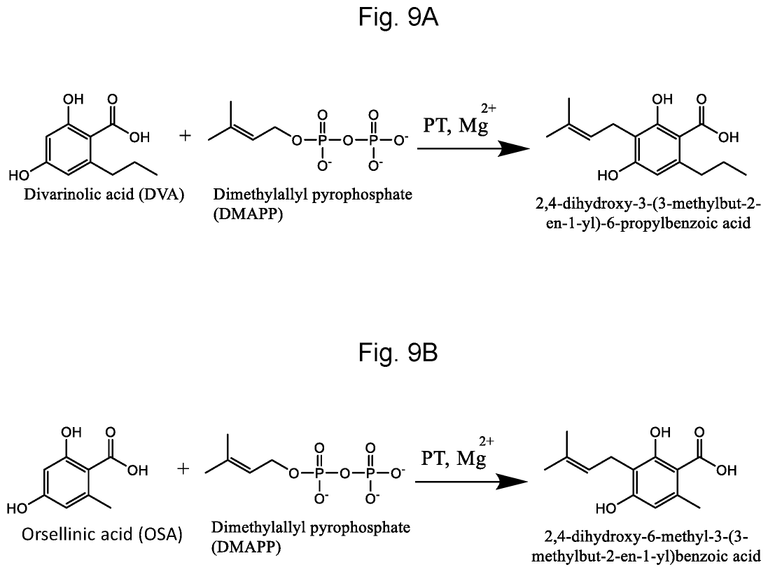

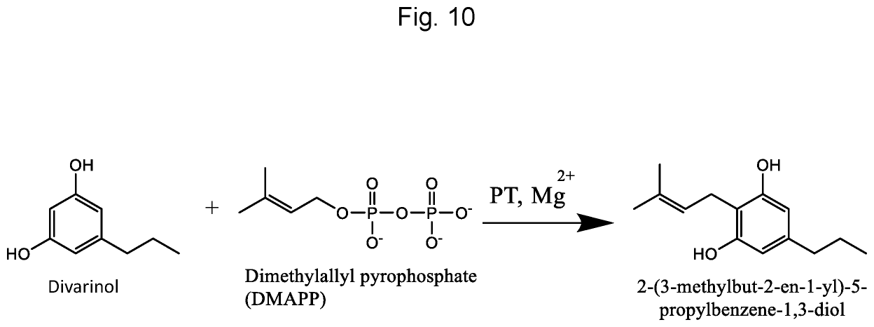

The disclosure also provides non-natural prenyltransferase variants enzymatically capable of regioselectively forming a 2-prenylated 5-alkylbenzene-1,3-diol from geranyl pyrophosphate and 5-alkylbenzene-1,3-diol. In some cases, the 5-alkylbenzene-1,3-diol substrate can be olivetol and the 2-prenylated 5-alkylbenzene-1,3-diol can be cannabigerol (CBG; 2-GOL), for example, the reaction which is shown in FIG. 8.

Cannabigerolic acid (CBGA; CAS #25555-57-1) has the following chemical names: (E)-3-(3,7-dimethyl-2,6-octadienyl)-2,4-dihydroxy-6-pentylbenzoic acid, and 3-[(2E)-3,7-dimethylocta-2,6-dien-1-yl]-2,4-dihydroxy-6-pentylbenzoic acid, and the following chemical structure:

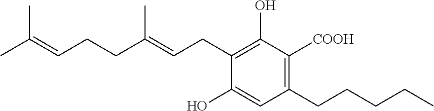 [SMILES: CCCCCc1cc(O)c(C/C=C(\C)CCC=C(C)C)c(O)c1C(=O)O]

CBGA can also be referred to as 3-geranyl-olivetolate (3-GOLA), which reflects the position of the geranyl moiety on the olivetolate moiety.

5-geranyl-olivetolate (5-GOLA) is an enzymatic reaction product of geranyl pyrophosphate and olivetolic acid and has the following structure.

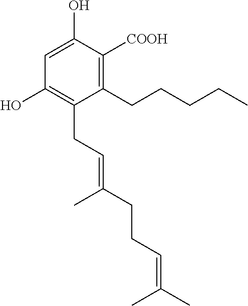 [SMILES: CCCCCc1c(C/C=C(\C)CCC=C(C)C)c(O)cc(O)c1C(=O)O]

As used herein, “geranyl-olivetolate” refers to either 3-GOLA or 5-GOLA. In an enzymatic reaction using a prenyltransferase variant, “geranyl-olivetolate” products (or “prenylation products”) can be produced, although for variants having high regioselectivity to 3-GOLA, very little or trace amounts of 5-GOLA may be produced.

Cannabigerol, the decarboxylated form of 3-GOLA, has the following structure.

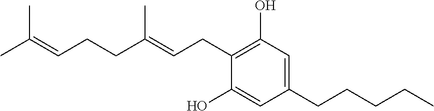 [SMILES: CCCCCc1cc(O)c(C/C=C(\C)CCC=C(C)C)c(O)c1]

Cannabigerol (CBG; 2-GOL; 2-[(2E)-3,7-dimethylocta-2,6-dienyl]-5-pentylbenzene-1.3-diol; CAS #: 25654-31-3) can be considered a “derivative” of GOLA/CBGA. 4-GOL is the isomer of 2-GOL prenylated at the 4 position on the aromatic ring (e.g., between a hydroxyl group at the 1 or 3 position on the aromatic ring and the pentyl group). CBG can be formed by decarboxylation of CBGA, for example by heat or by catalysis, which can be a biocatalyst such as an enzyme, whole cell, or cell extract. In addition to the use of olivetolic acid (OLA) for forming 3-GOLA/CBGA by reaction as catalyzed by prenyltransferase (see FIG. 1), the disclosure also contemplates the use of other substrate molecules as a replacement to OLA.

Cannabigerol (CBG; 2-GOL) can also be regioselectively formed (e.g., over formation of 4-GOL) from olivetol and geranyl pyrophosphate (see FIG. 8) using non-natural prenyltransferase variants of the disclosure.

Cannabigerovarinic acid (CBGVA; 3-GDVA; 3-[(2E)-3,7-dimethylocta-2,6-dienyl]-2.4-dihydroxy-6-propylbenzoic acid; C₂₀H₂₈O₄; #64924-07-8) is a minor cannabinoid.

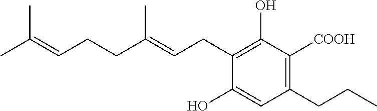 [SMILES: CCCc1cc(O)c(C/C=C(\C)CCC=C(C)C)c(O)c1C(=O)O]

5-GDVA is the isomeric form with the (2E)-3,7-dimethylocta-2,6-dienyl group attached to the 5 position on the aromatic ring. FIG. 7A shows reaction of divarinolic acid (DVA) and geranyl diphosphate (GPP) to form the product cannabigerovarinic acid (CBGVA).

Cannabigerorcinic acid (CBGOA; 3-GOSA; 3-[(2E)-3,7-dimethyl-2,6-octadien-1-yl]-2,4-dihydroxy-6-methyl-benzoic acid; C₁₈H₂₄O₄; #69734-83-4) is another minor cannabinoid.

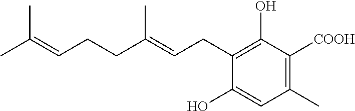 [SMILES: CC(C)=CCC/C(C)=C/Cc1c(O)cc(C)c(C(=O)O)c1O]

5-GOSA is the isomeric form with the (2E)-3,7-dimethyl-2,6-octadien-1-yl group attached to the 5 position on the aromatic ring. FIG. 7B shows reaction of orsellinic acid (OSA) and geranyl diphosphate (GPP) to form the product cannabigerorcinic acid (CBGOA).

The term “regioselective” and “regioselectivity” as used in a “regioselective reaction” refers to a direction of bond making or breaking that occurs preferentially over all other possible directions. A reaction between substrate A and substrate B may yield two or more reaction products (e.g., product C, product D, etc.). Regioselectivity can be understood by determining the molar amount of products formed. For example, in an enzymatic reaction wherein substrate A and substrate B react to form a product mixture of product C and product D, and wherein the molar ratio of product C:product D is greater than 1:1, respectively, in the product mixture, the reaction is regioselective to product C. When the molar ratio of product C:product D is 9:1 or greater, respectively, in the product mixture, the reaction has 90% or greater regioselectivity to product C.

The disclosure also contemplates methods for, generally, forming a prenylated aromatic compound. The method involves contacting a hydrophobic substrate and an aromatic substrate with a non-natural prenyltransferase of the disclosure to form a prenylated aromatic compound. For example, in particular, the disclosure contemplates use of various aromatic substrates such as olivetol, olivetolic acid, divarinol, divarinolic acid, orcinol, and orsellinic acid in such a prenyltransferase-catalyzed reaction. The hydrophobic substrate can include an isoprenoid portion, a geranyl portion, a farnesyl portions, a dimethylallyl portion and one or more phosphate groups. The method can be performed in vivo (e.g., within the engineered cell) or in vitro.

Also described are engineered cells expressing a non-natural prenyltransferase, optionally including one or more additional metabolic pathway transgene(s); cell culture compositions including the cells; methods for promoting production of the target cannabinoid or derivative thereof from the cells; compositions including the target cannabinoid or derivative; and products made from the target product or intermediate.

The term “non-naturally occurring”, when used in reference to an organism (e.g., microbial) is intended to mean that the organism has at least one genetic alteration not normally found in a naturally occurring organism of the referenced species. Naturally-occurring organisms can be referred to as “wild-type” such as wild type strains of the referenced species. Likewise, a “non-natural” polypeptide or nucleic acid can include at least one genetic alteration not normally found in a naturally-occurring polypeptide or nucleic acid. Naturally-occurring organisms, nucleic acids, and polypeptides can be referred to as “wild-type” or “original” such as wild type strains of the referenced species. Likewise, amino acids found in polypeptides of the wild type organism can be referred to as “original” with regards to any amino acid position.

A genetic alteration that makes an organism non-natural can include, for example, modifications introducing expressible nucleic acids encoding metabolic polypeptides, other nucleic acid additions, nucleic acid deletions and/or other functional disruption of the organism's genetic material. Such modifications include, for example, coding regions and functional fragments thereof, for heterologous, homologous or both heterologous and homologous polypeptides for the referenced species. Additional modifications include, for example, non-coding regulatory regions in which the modifications alter expression of a gene or operon.

For example, in order to provide a soluble aromatic prenyltransferase variant, a soluble ABBA type prenyltransferase from Streptomyces sp. CL190 (NCBI Accession number BAE00106.1; NphB; 307 amino acids long; SEQ ID NO: 1), can be selected as a template. Variants, as described herein, can be created by introducing into the template one or more amino acid substitutions to test for increased activity and improved regioselectivity to CBGA (3-GOLA).

For another example, in order to provide a soluble aromatic prenyltransferase variant, a soluble ABBA type prenyltransferase from Streptomyces antibioticus AQJ23 40425 (NCBI Accession number KUN17719.1; 305 amino acids long; SEQ ID NO: 16), can be selected as a template. Variants, as described herein, can be created by introducing into the template one or more amino acid substitutions to test for increased activity and improved regioselectivity to CBGA (3-GOLA).

In some cases, a “homolog” of the prenyltransferase SEQ ID NO:1 (NphB) is first identified. A homolog is a gene or genes that are related by vertical descent (i.e., orthologous) and are responsible for substantially similar or identical functions in different organisms. Genes are considered related by vertical descent when, for example, they share sequence similarity of sufficient amount to indicate they are homologous or related by evolution from a common ancestor. Genes that are orthologous can encode orthologous proteins (e.g., polypeptides) with sequence similarity of about 35% to 100% amino acid sequence identity, and more preferably about 60% to 100% amino acid sequence identity. Polypeptides can also be considered orthologs if they share three-dimensional structure but not necessarily significant sequence similarity, of a sufficient amount to indicate that they have evolved from a common ancestor to the extent that the primary sequence similarity is not identifiable. Paralogs are genes related by duplication within a genome, and can evolve new functions, even if these are related to the original one.

Polypeptides sharing a desired amount of identity (e.g., 35%, 45%, 50%, 55%, or 60% or greater) to the Streptomyces sp. CL190 prenyltransferase (NCBI Accession number BAE00106.1; NphB; SEQ ID NO: 1), including homologs, orthologs, and paralogs, can be determined by methods well known to those skilled in the art. For example, inspection of nucleic acid or amino acid sequences for two polypeptides may reveal sequence identity and similarities between the compared sequences. Based on such similarities, one skilled in the art can determine if the similarity is sufficiently high to indicate the proteins are related through evolution from a common ancestor.

Computational approaches to create multiple sequence alignment (MSA) and determination of sequence identity include global alignments and local alignments. Global alignment uses global optimization to force alignment to span the entire length of all query sequences. Local alignments, by contrast, identify regions of similarity within long sequences that are often widely divergent overall. For understanding the identity of a target sequence to the Streptomyces sp. CL190 prenyltransferase (NCBI Accession number BAE00106.1; NphB; SEQ ID NO: 1) template, a global alignment can be used. Optionally, amino terminal and/or carboxy-terminal sequences of the target sequence that share little or no identity with the template sequence can be excluded for a global alignment and generation of an identity score.

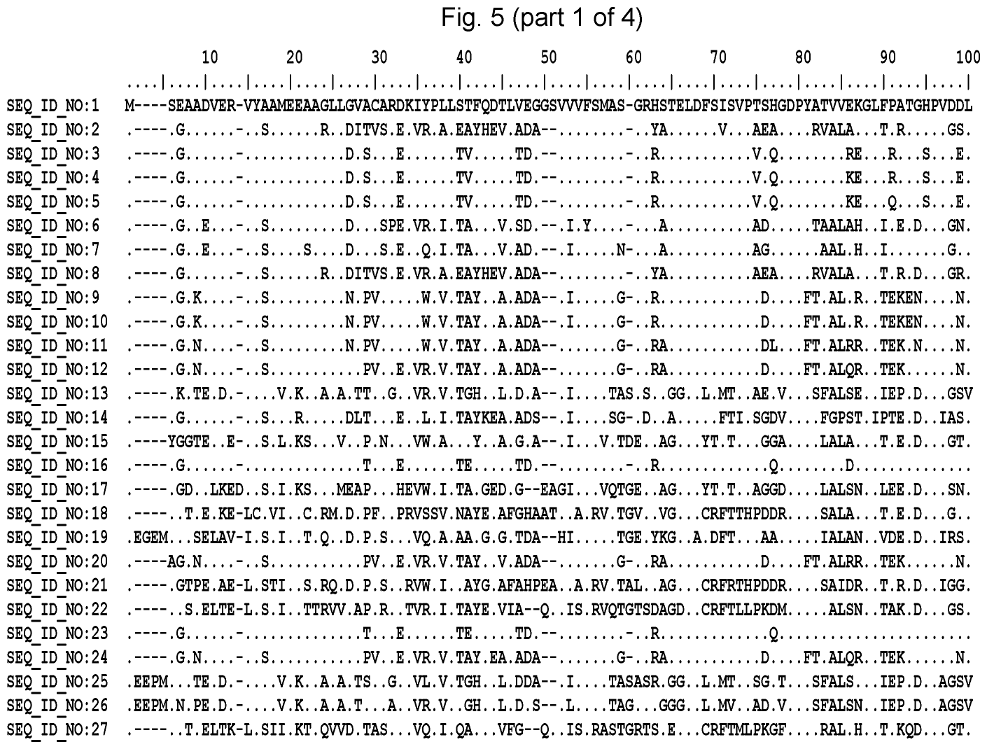

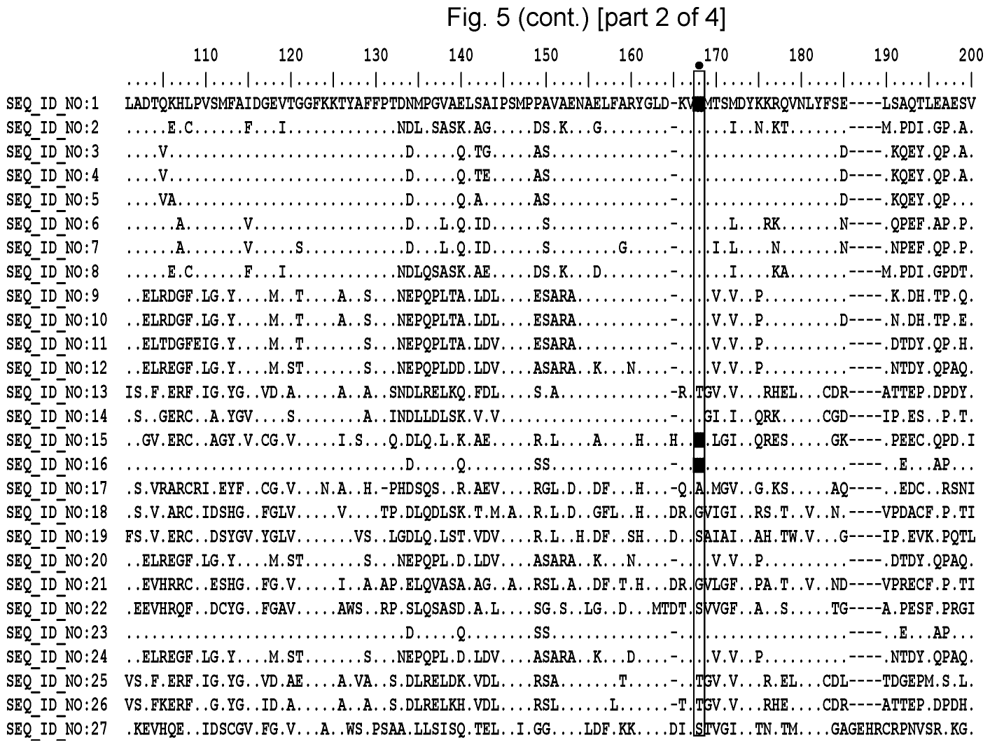

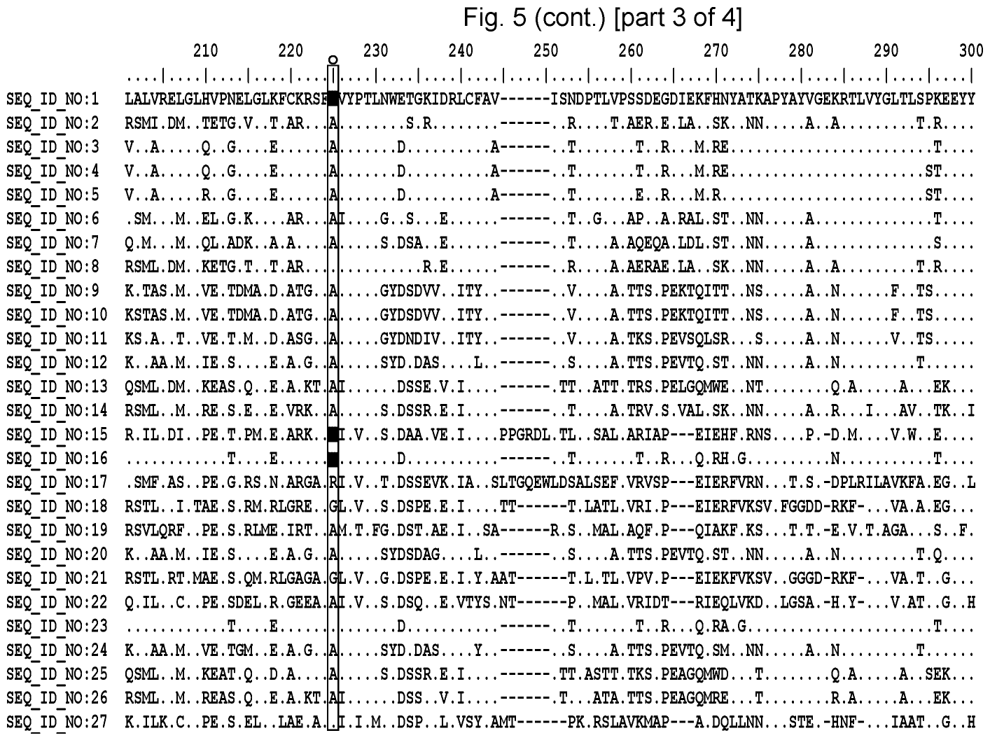

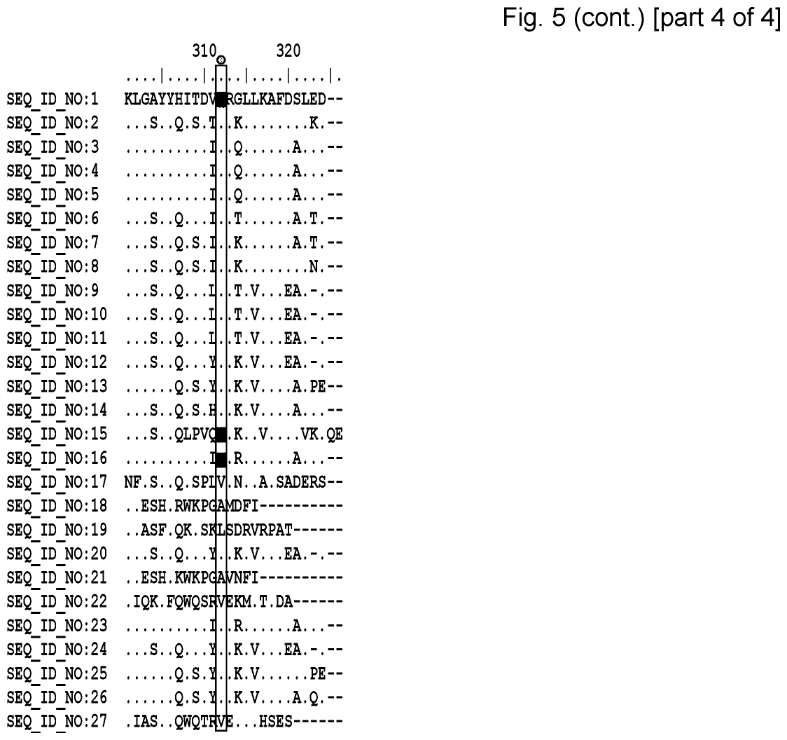

Algorithms well known to those skilled in the art, such as Align, BLAST, Clustal W and others compare and determine a raw sequence similarity or identity, and also determine the presence or significance of gaps in the sequence which can be assigned a weight or score. Such algorithms also are known in the art and are similarly applicable for determining nucleotide or amino acid sequence similarity or identity. Parameters for sufficient similarity to determine relatedness are computed based on well-known methods for calculating statistical similarity, or the chance of finding a similar match in a random polypeptide, and the significance of the match determined. A computer comparison of two or more sequences can, if desired, also be optimized visually by those skilled in the art. Related gene products or proteins can be expected to have a high similarity, for example, 35% to 100% sequence identity. Proteins that are unrelated can have an identity which is essentially the same as would be expected to occur by chance if a database of sufficient size is scanned (about 5%). Once a multiple sequence alignment (MSA) is established computationally and curated manually upon need by one skilled in the art (such as seen in FIG. 5), then amino acids found at a single alignment position in the MSA are recognized and considered as orthologous amino acid positions. These orthologous amino acid positions may serve similar roles structurally or functionally in the 3-dimensional polypeptide structure of the different respective homologs. It follows that identifying the effect of an amino acid substitution at a given position in one NphB homolog may predict similar effect in another related homolog at the same orthologous amino acid position. Importantly, the exact ordinal position of any two orthologous amino acids may not be identical between homologs. This is because the ordinal position number will shift from one homolog to the next depending on the presence of insertion or deletions (e.g., “indels”) of amino acid stretches of one orthologous amino acid or more found upstream of the given position in a comparison between different homologs.

Pairwise global sequence alignment can be carried out using Streptomyces sp. CL190 prenyltransferase (NCBI Accession number BAE00106.1; NphB; SEQ ID NO: 1) as the template. Alignment can be performed using the Needleman-Wunsch algorithm (Needleman, S. & Wunsch, C. A general method applicable to the search for similarities in the amino acid sequence of two proteins J. Mol. Biol, 1970, 48, 443-453) implemented through the BALIGN tool (balign.sourceforge.net). Default parameters are used for the alignment and BLOSUM62 was used as the scoring matrix. The disclosure also relates to the discovery of wild-type sequences disclosed herein as a prenyltransferase and as having improved activity as also described herein; such wild-type sequences previously annotated as “hypothetical protein” or “putative protein”. Based at least on identification, testing, identification of functionally important orthologous amino positions, and sequence alignments (see FIG. 5), the current disclosure further allows for the identification of prenyltransferases suitable for use in engineered cells and methods of the disclosure, such as creating variants as described herein.

For the purpose of amino acid position numbering, SEQ ID NO: 1 (NphB) is used as the reference sequence. For example, mention of orthologous amino acid position 51 is in reference to SEQ ID NO: 1 (NphB) but in the context of a different prenyltransferase sequence (a target sequence or other template sequence), the corresponding amino acid position for variant creation may have the same or different position number, (e.g., 48, 49 or 50). In some cases, the original amino acid and its position on the SEQ ID NO: 1 (NphB) reference template may precisely correlate with the original amino acid and position on the target prenyltransferase. In other cases, the original amino acid and its position on the SEQ ID NO: 1 (NphB) template may correlate with the original amino acid, but its position on the target may not be in the corresponding template position. However, the corresponding amino acid on the target can be a predetermined distance from the position on the template, such as: within greater than 10, 9, 8, 7, 6, 5, 4, 3, 2, or 1 amino acid positions from the template's position. In other cases, the original amino acid on the SEQ ID NO: 1 (NphB) template may not precisely correlate with the original amino acid on the target. However, one can understand what the corresponding amino acid on the target sequence is based on the general location of the amino acid on the template and the sequence of amino acids in the vicinity of the target amino acid, especially referring to the alignment provided in FIG. 5. It is understood that additional alignments can be generated with prenyltransferase sequences not specifically disclosed herein, and such alignments can be used to understand and generate new prenyltransferase variants in view of the current disclosure. In some cases, the alignments can allow one to understand common or similar amino acids in the vicinity of the target amino acid, and those amino acids may be viewed as orthologous amino acids positions between the template and target sequences.

In some cases, it can be useful to use the Basic Local Alignment Search Tool (BLAST) algorithm to understand the sequence identity between a template sequence (e.g., BLAST “query”) and a target sequence (e.g., BLAST “hit”). Therefore, in some aspects, BLAST is used to identify or understand the identity homologous and orthologous sequences. BLAST finds similar sequences using a heuristic method that approximates the Smith-Waterman algorithm by locating short matches between the two sequences. The (BLAST) algorithm can identify library sequences that resemble the query sequence above a certain threshold. Exemplary parameters for determining relatedness of two or more sequences using the BLAST algorithm, for example, can be as set forth below. Briefly, amino acid sequence alignments can be performed using BLASTP version 2.0.8 (Jan. 5, 1999) and the following parameters: Matrix: 0 BLOSUM62; gap open: 11; gap extension: 1; x_dropoff: 50; expect: 10.0; wordsize: 3; filter: on. Nucleic acid sequence alignments can be performed using BLASTN version 2.0.6 (Sep. 16, 1998) and the following parameters: Match: 1; mismatch: −2; gap open: 5; gap extension: 2; x_dropoff: 50; expect: 10.0; wordsize: 11; filter: off. Those skilled in the art will know what modifications can be made to the above parameters to either increase or decrease the stringency of the comparison, for example, and determine the relatedness of two or more sequences.

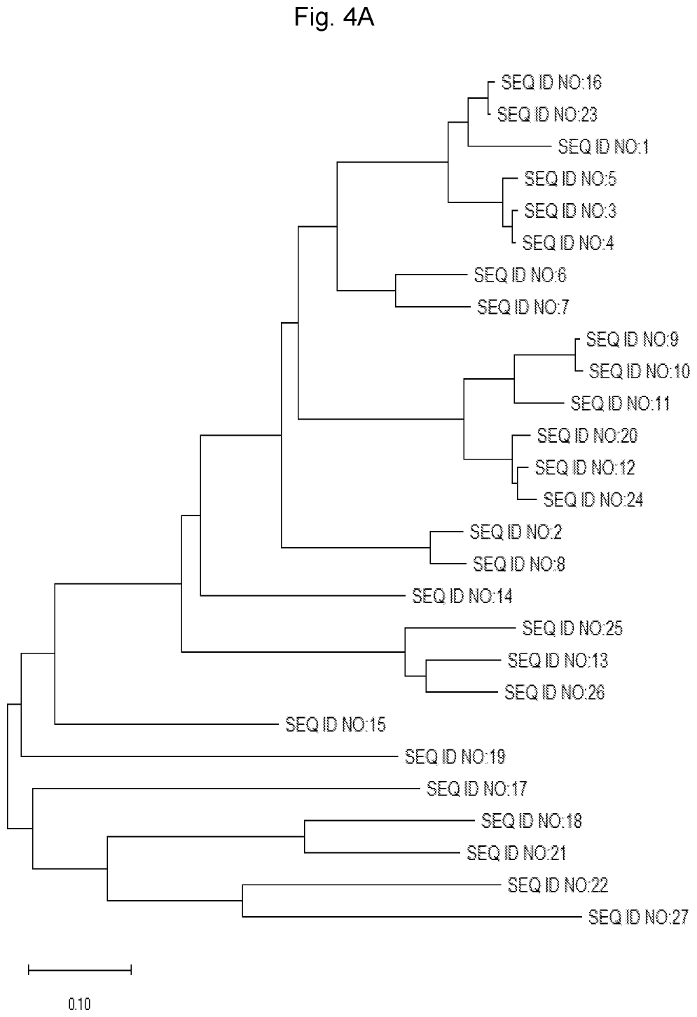

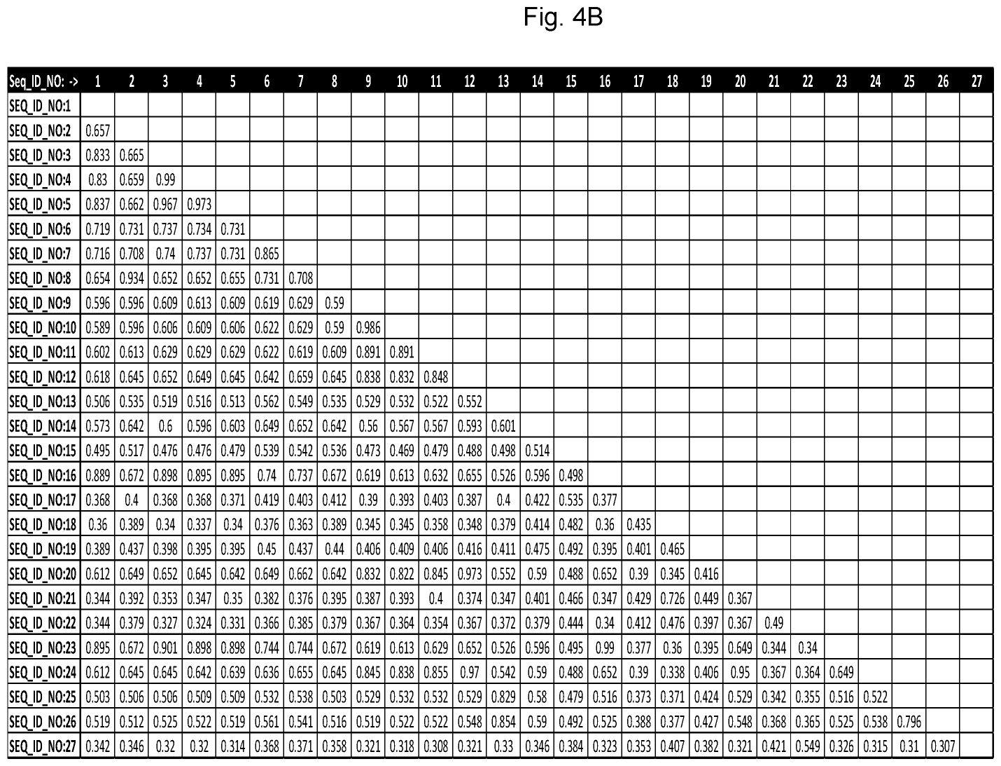

FIG. 5 shows an alignment of SEQ ID NO: 1 (Streptomyces sp. CL190; BAE00106.1; NphB) to other prenyltransferase homologs (SEQ ID NOs: 2-27). These homologs were found by BLAST search, and range in sequence identity to SEQ ID NO: 1 (NphB) from 89.5%-34.2% (SEQ ID NOs: 2-27) (FIG. 4B) (see also Table 1 for SEQ ID NOs: 1-27). Different homologs were tested for activity on OLA and GPP in cell lysate. Low, but measurable, activity was identified in a majority of the 27 homologs, with SEQ ID NO: 1 and SEQ ID NO: 16 among the most active enzymes. Low activities of wild-type homologs observed are in accord with that reported by Kumano, as previously mentioned.

TABLE 1
Amino acid sequences for NphB and
homologous prenyltransferases

| Accession No. | SEQ | |
|---|---|---|
| or | ID | |
| Descriptor | NO: | Amino Acid Sequence |
| BAE00106.1; | 1 | MSEAADVERVYAAMEEAAGLLGVAC |
| NphB; Orf2; | | ARDKIYPLLSTFQDTLVEGGSVVVF |
| 1ZB6A | | SMASGRHSTELDFSISVPTSHGDPY |
| | | ATVVEKGLFPATGHPVDDLLADTQK |
| | | HLPVSMFAIDGEVTGGFKKTYAFFP |
| | | TDNMPGVAELSAIPSMPPAVAENAE |
| | | LFARYGLDKVQMTSMDYKKRQVNLY |
| | | FSELSAQTLEAESVLALVRELGLHV |
| | | PNELGLKFCKRSFSVYPTLNWETGK |
| | | IDRLCFAVISNDPTLVPSSDEGDIE |
| | | KFHNYATKAPYAYVGEKRTLVYGLT |
| | | LSPKEEYYKLGAYYHITDVQRGLLK |
| | | AFDSLED |
| WP_101421563.1 | 2 | MSGAADVERVYSAMEEAARLLDITV |
| | | SREKVRPALEAYHEVLADAVVVFSM |
| | | ASGRYATELDFSVSVPAEAGDPYRV |
| | | ALAKGLTPRTGHPVGSLLADTQEHC |
| | | PVSMFAFDGEITGGFKKTYAFFPTN |
| | | DLPSASKLAGIPSMPDSVKENAGLF |
| | | ARYGLDKVQMTSIDYNKKTVNLYFS |
| | | EMSPDILGPEAVRSMIRDMGLTETG |
| | | EVGLTFARRSFAVYPTLNWESGRID |
| | | RLCFAVISRDPTLTPAEREEDLAKF |
| | | SKYANNAPYAYAGEARTLVYGLTLT |
| | | PREEYYKLGSYYQISDTQRKLLKAF |
| | | DSLKD |
| KPI30857.1 | 3 | MSGAADVERVYAAMEEAAGLLDVSC |
| | | AREKIYPLLTVFQDTLTDGVVVFSM |
| | | ASGRRSTELDFSISVPVSQGDPYAT |
| | | VVREGLFRATGSPVDELLADTVKHL |
| | | PVSMFAIDGEVTGGFKKTYAFFPTD |
| | | DMPGVAQLTGIPSMPASVAENAELF |
| | | ARYGLDKVQMTSMDYKKRQVNLYFS |
| | | DLKQEYLQPEAVVALARELGLQVPG |
| | | ELGLEFCKRSFAVYPTLNWDTGKID |
| | | RLCFAAISTDPTLVPSTDERDIEMF |
| | | REYATKAPYAYVGEKRTLVYGLTLS |
| | | PTEEYYKLGAYYHITDIQRQLLKAF |
| | | DALED |
| AFS18550.1 | 4 | MSGAADVERVYAAMEEAAGLLDVSC |
| | | AREKIYPLLTVFQDTLTDGVVVFSM |
| | | ASGRRSTELDFSISVPVSQGDPYAT |
| | | VVKEGLFRATGSPVDELLADTVKHL |
| | | PVSMFAIDGEVTGGFKKTYAFFPTD |
| | | DMPGVAQLTEIPSMPASVAENAELF |
| | | ARYGLDKVQMTSMDYKKRQVNLYFS |
| | | DLKQEYLQPEAVVALARELGLQVPG |
| | | ELGLEFCKRSFAVYPTLNWDTGKID |
| | | RLCFAAISTDPTLVPSTDERDIEMF |
| | | REYATKAPYAYVGEKRTLVYGLTLS |
| | | STEEYYKLGAYYHITDIQRQLLKAF |
| | | DALED |
| AWW43729.1 | 5 | MSGAADVERVYAAMEEAAGLLDVSCA |
| | | REKIYPLLTVFQDTLTDGVVVFSMA |
| | | SGRRSTELDFSISVPVSQGDPYATV |
| | | VKEGLFQATGSPVDELLADTVAHLP |
| | | VSMFAIDGEVTGGFKKTYAFFPTDD |
| | | MPGVAQLAAIPSMPASVAENAELFA |
| | | RYGLDKVQMTSMDYKKRQVNLYFSD |
| | | LKQEYLQPESWALARELGLRVPGEL |
| | | GLEFCKRSFAVYPTLNWDTGKIDRL |
| | | CFAAISTDPTLVPSEDERDIEMFRN |
| | | YATKAPYAYVGEKRTLVYGLTLSST |
| | | EEYYKLGAYYHITDIQRQLLKAFDA |
| | | LED |
| WP_073501789.1 | 6 | MSGAAEVERVYSAMEEAAGLLDVAC |
| | | SPEKVRPILTAFQDVLSDGVIVYSM |
| | | ASGRHATELDFSISVPADHGDPYTA |
| | | ALAHGLIPETDHPVGNLLADTQKAL |
| | | PVSMFAVDGEVTGGFKKTYAFFPTD |
| | | DMPGLAQLIDIPSMPPSVAENAELF |
| | | ARYGLDKVQMTSLDYKRKQVNLYFS |
| | | NLQPEFLAPEPVLSMVREMGLELPG |
| | | EKGLKFARRSFAIYPTLGWESGKIE |
| | | RLCFAVISTDPGLVPAPDEADRALF |
| | | STYANNAPYAYAGEKRTLVYGLTLS |
| | | PTEEYYKLGSYYQITDIQRTLLKAF |
| | | DALTD |
| WP_078582630.1 | 7 | MSGAAEVERVYSAMEESAGLLDVAC |
| | | SREKIQPILTAFQDVLADGVIVFSM |
| | | ANGRHATELDFSISVPAGHGDPYAA |
| | | ALEHGLIPATGHPVGDLLADTQKAL |
| | | PVSMFAVDGEVTSGFKKTYAFFPTD |
| | | DMPGLAQLIDIPSMPPSVAENAELF |
| | | GRYGLDKVQMISLDYKKNQVNLYFS |
| | | NLNPEFLQPEPVQAMVREMGLQLPA |
| | | DKGLAFAKRSFAVYPTLSWDSAKIE |
| | | RLCFAVISTDPTLAPAQEQADLDLF |
| | | STYANNAPYAYAGEKRTLVYGLTLS |
| | | PSEEYYKLGSYYQISDIQRKLLKAF |
| | | DALTD |
| WP_047018069.1 | 8 | MSGAADVERVYSAMEEAARLLDITV |
| | | SREKVRPALEAYHEVLADAVVVFSM |
| | | ASGRYATELDFSISVPAEAGDPYRV |
| | | ALAKGLTPRTDHPVGRLLADTQEHC |
| | | PVSMFAFDGEITGGFKKTYAFFPTN |
| | | DLQSASKLAEIPSMPDSVKENADLF |
| | | ARYGLDKVQMTSIDYKKKAVNLYFS |
| | | EMSPDILGPDTVRSMLRDMGLKETG |
| | | ETGLTFARRSFSVYPTLNWETGRIE |
| | | RLCFAVISRDPTLAPAERAEDLAKF |
| | | SKYANNAPYAYAGEARTLVYGLTLT |
| | | PREEYYKLGSYYQISDIQRKLLKAF |
| | | DSLND |
| Unknown ORF | 9 | MSGAKDVERVYSAMEEAAGLLNVPV |
| | | ARDKIWPVLTAYQDALADAVIVFSM |
| | | AGGRRSTELDFSISVPTDHGDPFTT |
| | | ALERGLTEKENHPVDNLLAELRDGF |
| | | PLGMYAIDGMVTTGFKKAYASFPTN |
| | | EPQPLTALLDLPSMPESARANAELF |
| | | ARYGLDKVQMVSVDYPKRQVNLYFS |
| | | ELKADHLTPEQVKATASEMGLVEPT |
| | | DMALDFATGSFAVYPTLGYDSDVVD |
| | | RITYAVISVDPTLAPTTSEPEKTQI |
| | | TTYANSAPYAYAGENRTLVYGFTLT |
| | | SKEEYYKLGSYYQITDLQRTLVKAF |
| | | EALD |
| WP_078627616.1 | 10 | MSGAKDVERVYSAMEEAAGLLNVPV |
| | | ARDKIWPVLTAYQDALADAVIVFSM |
| | | AGGRRSTELDFSISVPTDHGDPFTT |
| | | ALERGLTEKENHPVDNLLAELRDGF |
| | | PLGMYAIDGMVTTGFKKAYASFPTN |
| | | EPQPLTALLDLPSMPESARANAELF |
| | | ARYGLDKVQMVSVDYPKRQVNLYFS |
| | | DLNADHLTPEEVKSTASEMGLVEPT |
| | | DMALDFATGSFAVYPTLGYDSDVVD |
| | | RITYAVISVDPTLAPTTSEPEKTQI |
| | | TTYANSAPYAYAGENRTLVYGFTLT |
| | | SKEEYYKLGSYYQITDLQRTLVKAF |
| | | EALD |
| Unknown ORF | 11 | MSGANDVERVYSAMEEAAGLLNVPV |
| | | ARDKIWPVLTAYQDALADAVVVFSM |
| | | AGGRRATELDFSISVPTDLGDPFTT |
| | | ALRRGLTEKTNHPVDNLLAELTDGF |
| | | EIGMYAIDGMVTTGFKKTYASFPTN |
| | | EPQPLTALLDVPSMPESARANAELF |
| | | ARYGLDKVQMVSVDYPKRQVNLYFS |
| | | ELDTDYLQPEHVKSLARETGLVEPT |
| | | EMGLDFASGSFAVYPTLGYDNDIVD |
| | | RITYAVISVDPTLAPTKSEPEVSQL |
| | | SRYATSAPYAYAGENRTLVYGVTLT |
| | | SKEEYYKLGSYYQITDLQRTLVKAF |
| | | EALD |
| WP_052770383.1 | 12 | MSGANDVERVYSAMEEAAGLLGVPV |
| | | AREKVRPVLTAYQDALADAVVVFSM |
| | | AGGRRATELDFSISVPTDHGDPFTT |
| | | ALQRGLTEKTGHPVDNLLAELREGF |
| | | PLGMYAIDGMVSTGFKKTYASFPTN |
| | | EPQPLDDLLDVPSMPASARANAKLF |
| | | ANYGLDKVQMVSVDYPKRQVNLYFS |
| | | ELNTDYLQPAQVKALAAEMGLIEPS |
| | | ELGLEFAKGSFAVYPTLSYDTDASD |
| | | RLCLAVISSDPTLAPTTSEPEVTQF |
| | | STYANNAPYAYAGENRTLVYGLTLT |
| | | PKEEYYKLGSYYQITDYQRKLVKAF |
| | | EALD |
| WP 078616106.1 | 13 | MSKATEVDRVYAAVEKAAALAGTTC |
| | | AGDKVRPVLTGHQDLLDEAVIVFSM |
| | | TASGSHSGGLDLSMTVPAEHVDPYS |
| | | FALSEGLIEPTDHPVGSVISDFQER |
| | | FPIGMYGIDVDVAGGFKKAYAAFPS |
| | | NDLRELKQLFDLPSMPSAAAENAEL |
| | | FARYGLDRVTGVSVDYKRHELNLYC |
| | | DRATTEPLDPDYVQSMLRDMGLKEA |
| | | SEQGLEFAKKTFAIYPTLNWDSSEI |
| | | VRICFAVITTDPATTPTRSEPELGQ |
| | | MWEYANTAPYAYVGEQRALVYGLAL |
| | | SPEKEYYKLGAYYQISDYQRKLVKA |
| | | FDALPE |
| AQU65790.1 | 14 | MSGAADVERVYSAMERAAGLLDLTC |
| | | AREKILPILTAYKEALADSVIVFSM |
| | | SGGDHSAELDFSFTIPSGDVDPYAF |
| | | GPSTGIPTETDHPIASLLSDTGERC |
| | | PVAMYGVDGEVSGGFKKTYAAFPIN |
| | | DLLDLSKLVAVPSMPPAVAENAELF |
| | | ARYGLDKVQGISIDYQRKQVNLYCG |
| | | DIPAESLEPETVRSMLREMGLREPS |
| | | EEGLEFVRKSFAVYPTLSWDSSRIE |
| | | RICFAVISTDPTLAPTRVESDVALF |
| | | SKYANNAPYAYAGERRTLIYGLAVS |
| | | PTKEYIKLGSYYQISDHQRKLVKAF |
| | | DALED |
| WP_027748955.1 | 15 | MYGGTEVEEVYSALEKSAGLVGVPC |
| | | NRDKVWPALSTYQDALGEAVIVFSV |
| | | ATDERHAGELDYTITVPTGGADPYA |
| | | LALAKGLTPETDHPVGTLLAGVQER |
| | | CPVAGYAVDCGVVGGFKKIYSFFPQ |
| | | DDLQGLAKLAEIPSMPRALAENAAL |
| | | FARHGLDHKVTMLGIDYQRESVNLY |
| | | FGKLPEECLQPDSIRAILRDIGLPE |
| | | PTEPMLEFARKSFAIYVTLSWDAAK |
| | | VERICFAVPPGRDLITLDPSALPAR |
| | | IAPEIEHFARNSPYAYPGDRMLVYG |
| | | VTWSPEEEYYKLGSYYQLPVQTRKL |
| | | LVAFDSVKDQE |
| KUN17719.1 | 16 | MSGAADVERVYAAMEEAAGLLGVTC |
| | | AREKIYPLLTEFQDTLTDGVVVFSM |
| | | ASGRRSTELDFSISVPTSQGDPYAT |
| | | VVDKGLFPATGHPVDDLLADTQKHL |
| | | PVSMFAIDGEVTGGFKKTYAFFPTD |
| | | DMPGVAQLSAIPSMPSSVAENAELF |
| | | ARYGLDKVQMTSMDYKKRQVNLYFS |
| | | ELSEQTLAPESVLALVRELGLHVPT |
| | | ELGLEFCKRSFSVYPTLNWDTGKID |
| | | RLCFAVISTDPTLVPSTDERDIEQF |
| | | RHYGTKAPYAYVGENRTLVYGLTLS |
| | | PTEEYYKLGAYYHITDIQRRLLKAF |
| | | DALED |
| WP_130468244.1 | 17 | MSGDADLKEDVYSAIEKSAGLMEAP |
| | | CARHEVWPILTAFGEDLGEAGIVFS |
| | | VQTGERHAGELDYTITVPAGGDDPY |
| | | ALALSNGLLEETDHPVSNLLSDVRA |
| | | RCRISEYFIDCGVVGGFNKAYAHFP |
| | | HDSQSVARLAEVPSMPRGLADNADF |
| | | FARHGLDQVAMMGVDYGKKSVNLYF |
| | | AQLSEDCLERSNILSMFRASGLPEP |
| | | GERSLNFARGAFRIYVTLTWDSSEV |
| | | KRIAFASLTGQEWLDSALSEFPVRV |
| | | SPEIERFVRNAPYTYSGDPLRILAV |
| | | KFAPEGEYLNFGSYYQISPLVRNLL |
| | | AASADERS |
| WP_117400900.1 | 18 | MSETAEVKELCAVIEECARMLDVPF |
| | | ARPRVSSVLNAYEDAFGHAATVVAF |
| | | RVATGVRHVGELDCRFTTHPDDRDP |
| | | YASALAKGLTPETDHPVGDLLSDVQ |
| | | ARCPIDSHGIDFGLVGGFKKVYAFF |
| | | TPDDLQDLSKLTAMPAMPRALADNA |
| | | GFLARHGLDDRVGVIGIDYRSRTVN |
| | | VYFNEVPDACFEPETIRSTLREIGT |
| | | AEPSERMLRLGRESFGLYVTLSWDS |
| | | PEIERICFAVTTTDLATLPVRIEPE |
| | | IERFVKSVPFGGDDRKFVYGVALAP |
| | | EGEYYKLESHYRWKPGAMDFI |
| CDH35382.1 | 19 | MEGEMSEASELAVIYSAIEETAQLL |
| | | DVPCSRDKVQPALAAFGDGLTDAHI |
| | | VFSMATGERYKGELAFDFTVPTAAG |
| | | DPYAIALANGLVDETDHPIRSLFSD |
| | | VQERCPVDSYGVDYGLVGGFKKTYV |
| | | SFPLGDLQGLSTLVDVPSMPRALAE |
| | | HADFFASHGLDDKVSAIAIDYAHRT |
| | | WNVYFSGIPAEVKEPQTLRSVLQRF |
| | | GLPEPSERLMEFIRTSFAMYTTFGW |
| | | DSTKAERICFSARSSDPMALPAQFE |
| | | PQIAKFAKSAPYTYTGERVLTYAGA |
| | | LSPSEEFYKLASFYQKTSKLSDRVR |
| | | PAT |
| WP_125936269.1 | 20 | MAGANDVERVYSAMEEAAGLLGVPV |
| | | AREKVRPVLTAYQDVLADAVVVFSM |
| | | AGGRRATELDFSISVPTDHGDPFTT |
| | | ALRRGLTEKTGHPVDNLLAELREGF |
| | | PLGMYAIDGMVSTGFKKTYASFPTN |
| | | EPQPLADLLDVPSMPASARANAKLF |
| | | ANYGLDKVQMVSVDYPKRQVNLYFS |
| | | ELDTDYLQPAQVKALAAEMGLIEPS |
| | | ELGLEFAKGSFAVYPTLSYDSDAGD |
| | | RLCLAVISSDPTLAPTTSEPEVTQF |
| | | STYANNAPYAYAGENRTLVYGLTLT |
| | | PQEEYYKLGSYYQITDYQRKLVKAF |
| | | EALD |
| WP_03 0499012.1 | 21 | MSGTPEVAELYSTIEESARQLDVPC |
| | | SRDRVWPILSAYGDAFAHPEAVVAF |
| | | RVATALRHAGELDCRFRTHPDDRDP |
| | | YASAIDRGLTPRTDHPIGGLLAEVH |
| | | RRCPVESHGIDFGVVGGFKKIYAAF |
| | | APDELQVASALAGIPAMPRSLAANA |
| | | DFFTRHGLDDRVGVLGFDYPARTVN |
| | | VYFNDVPRECFEPETIRSTLRRTGM |
| | | AEPSEQMLRLGAGAFGLYVTLGWDS |
| | | PEIERICYAAATTDLTTLPVPVEPE |
| | | IEKFVKSVPYGGGDRKFVYGVALTP |
| | | KGEYYKLESHYKWKPGAVNFI |
| C4PWA1.1 | 22 | MSESAELTELYSAIEETTRVVGAPC |
| | | RRDTVRPILTAYEDVIAQSVISFRV |
| | | QTGTSDAGDLDCRFTLLPKDMDPYA |
| | | TALSNGLTAKTDHPVGSLLEEVHRQ |
| | | FPVDCYGIDFGAVGGFKKAWSFFRP |
| | | DSLQSASDLAALPSMPSGVSENLGL |
| | | FDRYGMTDTVSVVGFDYAKRSVNLY |
| | | FTGASPESFEPRGIQAILRECGLPE |
| | | PSDELLRFGEEAFAIYVTLSWDSQK |
| | | IERVTYSVNTPDPMALPVRIDTRIE |
| | | QLVKDAPLGSAGHRYVYGVTATPKG |
| | | EYHKIQKYFQWQSRVEKMLTADA |
| WP_109499705.1 | 23 | MSGAADVERVYAAMEEAAGLLGVTC |
| | | AREKIYPLLTEFQDTLTDGVVVFSM |
| | | ASGRRSTELDFSISVPTSQGDPYAT |
| | | VVEKGLFPATGHPVDDLLADTQKHL |
| | | PVSMFAIDGEVTGGFKKTYAFFPTD |
| | | DMPGVAQLSAIPSMPSSVAENAELF |
| | | ARYGLDKVQMTSMDYKKRQVNLYFS |
| | | ELSEQTLAPESVLALVRELGLHVPT |
| | | ELGLEFCKRSFSVYPTLNWDTGKID |
| | | RLCFAVISTDPTLVPSTDERDIEQF |
| | | RAYGTKAPYAYVGEKRTLVYGLTLS |
| | | PTEEYYKLGAYYHITDIQRRLLKAF |
| | | DALED |
| WP_101422827.1 | 24 | MSGANDVERVYSAMEEAAGLLGVPV |
| | | AREKVRPVLTAYQEALADAWVFSMA |
| | | GGRRATELDFSISVPTDHGDPFTTA |
| | | LQRGLTEKTGHPVDNLLAELREGFP |
| | | LGMYAIDGMVSTGFKKTYASFPTNE |
| | | PQPLADLLDVPSMPASARANAKLFA |
| | | DYGLDKVQMVSVDYPKRQVNLYFSE |
| | | LNTDYLQPAQVKALAAEMGLVEPTG |
| | | MGLEFAKGSFAVYPTLSYDTDASDR |
| | | LCYAVISSDPTLAPTTSEPEVTQFS |
| | | MYANNAPYAYAGENRTLVYGLTLTP |
| | | KEEYYKLGSYYQITDYQRKLVKAFE |
| | | ALD |
| WP_103783028.1 | 25 | MEEPMSEATEVDRVYAAVEKAAALA |
| | | GTSCAGDKVLPVLTGHQDLLDDAVI |
| | | VFSMTASASRSGGLDLSMTVPSGHT |
| | | DPYSFALSKGLIEPTDHPAGSVVSD |
| | | FQERFPIGMYGIDVDVAEGFKKAYV |
| | | AFPSDDLRELDKLVDLPSMPRSAAE |
| | | NAELFTRYGLDKVTGVSVDYKRREL |
| | | NLYCDLTDGEPMESELVQSMLREMG |
| | | LKEATEQGLDFAKRSFAVYPTLSWD |
| | | SSRIERICFAVITTDASTTPTKSEP |
| | | EAGQMWDYATTAPYAYVGEQRALVY |
| | | GLALSSEKEYYKLGAYYQISDYQRK |
| | | LVKAFDSLPE |
| WP_143644462.1 | 26 | MEEPMSNAPEVDRVYAAVEKAAALA |
| | | GTACAADKVRPVLSGHQDLLDESVL |
| | | VFSMTAGGRHGGGLDLSMVVPADHV |
| | | DPYSFALSNGLIEPTDHPAGSVVSD |
| | | FKERFPVGMYGIDIDVAGGFKKAYA |
| | | AFPSDDLRELKHLVDLPSMPRSLAE |
| | | NAELLARYGLDTVTGVSVDYKRHEV |
| | | NLYCDRATTEPLDPDHVRSMLREMG |
| | | LREASEQGLEFAKKTFAIYPTLNWD |
| | | SSKIVRICFAVITNDPATAPTTSEP |
| | | EAGQMREYATTAPYAYVGERRALVY |
| | | GLALSPEKEYYKLGAYYQISDYQRK |
| | | LVKAFDALQD |
| AEW22941.1 | 27 | MSETAELTKLYSIIEKTAQVVDVTA |
| | | SRDKVQPILQAFQDVFGQSVISFRA |
| | | STGRTSSEELDCRFTMLPKGFDPYA |
| | | RALEHGLTPKQDHPVGTLLKEVHQE |
| | | LPIDSCGVDFGVVGGFAKTWSFPSA |
| | | ANLLSISQLTELPSIPGGVAENLDF |
| | | FKKYGLDDIVSTVGIDYTNRTMNLY |
| | | FGAGEHRCRPNVSRAKGVKAILKEC |
| | | GLPEPSEELLKLAERAFSIYITMNW |
| | | DSPKILRVSYAAMTPKPRSLAVKMA |
| | | PAFDQLLNNAPYSTEGHNFVYGIAA |
| | | TPKGEYHKIASYYQWQTRVEGLLHS |
| | | ES |
| Q161H + Q295W | 28 | MSGAADVERVYAAMEEAAGLLGVTC |
| INS45 on | | AREKIYPLLTEFQDTLTDGGSVVVF |
| (scaffold) | | SMASGRRSTELDFSISVPTSQGDPY |
| SEQ ID NO: 16 | | ATVVDKGLFPATGHPVDDLLADTQK |
| | | HLPVSMFAIDGEVTGGFKKTYAFFP |
| | | TDDMPGVAQLSAIPSMPSSVAENAE |
| | | LFARYGLDKVHMTSMDYKKRQVNLY |
| | | FSELSEQTLAPESVLALVRELGLHV |
| | | PTELGLEFCKRSFSVYPTLNWDTGK |
| | | IDRLCFAVISTDPTLVPSTDERDIE |
| | | QFRHYGTKAPYAYVGENRTLVYGLT |
| | | LSPTEEYYKLGAYYHITDIWRRLLK |
| | | AFDALED |
| Q161S + Q295W | 29 | MSGAADVERVYAAMEEAAGLLGVTC |
| INS45 | | AREKIYPLLTEFQDTLTDGGSVVVF |
| on | | SMASGRRSTELDFSISVPTSQGDPY |
| (scaffold) | | ATVVDKGLFPATGHPVDDLLADTQK |
| SEQ ID NO: 16 | | HLPVSMFAIDGEVTGGFKKTYAFFP |
| | | TDDMPGVAQLSAIPSMPSSVAENAE |
| | | LFARYGLDKVSMTSMDYKKRQVNLY |
| | | FSELSEQTLAPESVLALVRELGLHV |
| | | PTELGLEFCKRSFSVYPTLNWDTGK |
| | | IDRLCFAVISTDPTLVPSTDERDIE |
| | | QFRHYGTKAPYAYVGENRTLVYGLT |
| | | LSPTEEYYKLGAYYHITDIWRRLLK |
| | | AFDALED |
| Q159S + S212H | 30 | MSGAADVERVYAAMEEAAGLLGVTC |
| on | | AREKIYPLLTEFQDTLTDGVVVFSM |
| (scaffold) | | ASGRRSTELDFSISVPTSQGDPYAT |
| SEQ ID NO: 16 | | VVDKGLFPATGHPVDDLLADTQKHL |
| | | PVSMFAIDGEVTGGFKKTYAFFPTD |
| | | DMPGVAQLSAIPSMPSSVAENAELF |
| | | ARYGLDKVSMTSMDYKKRQVNLYFS |
| | | ELSEQTLAPESVLALVRELGLHVPT |
| | | ELGLEFCKRSFHVYPTLNWDTGKID |
| | | RLCFAVISTDPTLVPSTDERDIEQF |
| | | RHYGTKAPYAYVGENRTLVYGLTLS |
| | | PTEEYYKLGAYYHITDIQRRLLKAF |
| | | DALED |
| Q159S + Y286V | 31 | MSGAADVERVYAAMEEAAGLLGVTC |
| on | | AREKIYPLLTEFQDTLTDGVVVFSM |
| (scaffold) | | ASGRRSTELDFSISVPTSQGDPYAT |
| SEQ ID NO: 16 | | VVDKGLFPATGHPVDDLLADTQKHL |
| | | PVSMFAIDGEVTGGFKKTYAFFPTD |
| | | DMPGVAQLSAIPSMPSSVAENAELF |
| | | ARYGLDKVSMTSMDYKKRQVNLYFS |
| | | ELSEQTLAPESVLALVRELGLHVPT |
| | | ELGLEFCKRSFSVYPTLNWDTGKID |
| | | RLCFAVISTDPTLVPSTDERDIEQF |
| | | RHYGTKAPYAYVGENRTLVYGLTLS |
| | | PTEEYYKLGAVYHITDIQRRLLKAF |
| | | DALED |
| S212H + Y286V | 32 | MSGAADVERVYAAMEEAAGLLGVTC |
| on | | AREKIYPLLTEFQDTLTDGVVVFSM |
| (scaffold) | | ASGRRSTELDFSISVPTSQGDPYAT |
| SEQ ID NO: 16 | | VVDKGLFPATGHPVDDLLADTQKHL |
| | | PVSMFAIDGEVTGGFKKTYAFFPTD |
| | | DMPGVAQLSAIPSMPSSVAENAELF |
| | | ARYGLDKVQMTSMDYKKRQVNLYFS |
| | | ELSEQTLAPESVLALVRELGLHVPT |
| | | ELGLEFCKRSFHVYPTLNWDTGKID |
| | | RLCFAVISTDPTLVPSTDERDIEQF |
| | | RHYGTKAPYAYVGENRTLVYGLTLS |
| | | PTEEYYKLGAVYHITDIQRRLLKAF |
| | | DALED |
| Q161S + INS45 | 33 | MSGAADVERVYAAMEEAAGLLGVTC |
| on | | AREKIYPLLTEFQDTLTDGGSVVVF |
| (scaffold) | | SMASGRRSTELDFSISVPTSQGDPY |
| SEQ ID NO: 16 | | ATVVDKGLFPATGHPVDDLLADTQK |
| | | HLPVSMFAIDGEVTGGFKKTYAFFP |
| | | TDDMPGVAQLSAIPSMPSSVAENAE |
| | | LFARYGLDKVSMTSMDYKKRQVNLY |
| | | FSELSEQTLAPESVLALVRELGLHV |
| | | PTELGLEFCKRSFSVYPTLNWDTGK |
| | | IDRLCFAVISTDPTLVPSTDERDIE |
| | | QFRHYGTKAPYAYVGENRTLVYGLT |
| | | LSPTEEYYKLGAYYHITDIQRRLLK |
| | | AFDALED |
| S214H + INS45 | 34 | MSGAADVERVYAAMEEAAGLLGVTC |
| on | | AREKIYPLLTEFQDTLTDGGSVVVF |
| (scaffold) | | SMASGRRSTELDFSISVPTSQGDPY |
| SEQ ID NO: 16 | | ATVVDKGLFPATGHPVDDLLADTQK |
| | | HLPVSMFAIDGEVTGGFKKTYAFFP |
| | | TDDMPGVAQLSAIPSMPSSVAENAE |
| | | LFARYGLDKVQMTSMDYKKRQVNLY |
| | | FSELSEQTLAPESVLALVRELGLHV |
| | | PTELGLEFCKRSFHVYPTLNWDTGK |
| | | IDRLCFAVISTDPTLVPSTDERDIE |
| | | QFRHYGTKAPYAYVGENRTLVYGLT |
| | | LSPTEEYYKLGAYYHITDIQRRLLK |
| | | AFDALED |
| Y288V + INS45 | 35 | MSGAADVERVYAAMEEAAGLLGVTC |
| on | | AREKIYPLLTEFQDTLTDGGSVVVF |
| (scaffold) | | SMASGRRSTELDFSISVPTSQGDPY |
| SEQ ID NO: 16 | | ATVVDKGLFPATGHPVDDLLADTQK |
| | | HLPVSMFAIDGEVTGGFKKTYAFFP |
| | | TDDMPGVAQLSAIPSMPSSVAENAE |
| | | LFARYGLDKVQMTSMDYKKRQVNLY |
| | | FSELSEQTLAPESVLALVRELGLHV |
| | | PTELGLEFCKRSFSVYPTLNWDTGK |
| | | IDRLCFAVISTDPTLVPSTDERDIE |
| | | QFRHYGTKAPYAYVGENRTLVYGLT |
| | | LSPTEEYYKLGAVYHITDIQRRLLK |
| | | AFDALED |
| Q159H + Q293W | 36 | MSGAADVERVYAAMEEAAGLLGVTC |
| on | | AREKIYPLLTEFQDTLTDGVVVFSM |
| (scaffold) | | ASGRRSTELDFSISVPTSQGDPYAT |
| SEQ ID NO: 16 | | VVDKGLFPATGHPVDDLLADTQKHL |
| | | PVSMFAIDGEVTGGFKKTYAFFPTD |
| | | DMPGVAQLSAIPSMPSSVAENAELF |
| | | ARYGLDKVHMTSMDYKKRQVNLYFS |
| | | ELSEQTLAPESVLALVRELGLHVPT |
| | | ELGLEFCKRSFSVYPTLNWDTGKID |
| | | RLCFAVISTDPTLVPSTDERDIEQF |
| | | RHYGTKAPYAYVGENRTLVYGLTLS |
| | | PTEEYYKLGAYYHITDIWRRLLKAF |
| | | DALED |
| Q161H + INS45 | 37 | MSGAADVERVYAAMEEAAGLLGVTC |
| on | | AREKIYPLLTEFQDTLTDGGSVVVF |
| (scaffold) | | SMASGRRSTELDFSISVPTSQGDPY |
| SEQ ID NO: 16 | | ATVVDKGLFPATGHPVDDLLADTQK |
| | | HLPVSMFAIDGEVTGGFKKTYAFFP |
| | | TDDMPGVAQLSAIPSMPSSVAENAE |
| | | LFARYGLDKVHMTSMDYKKRQVNLY |
| | | FSELSEQTLAPESVLALVRELGLHV |
| | | PTELGLEFCKRSFSVYPTLNWDTGK |
| | | IDRLCFAVISTDPTLVPSTDERDIE |
| | | QFRHYGTKAPYAYVGENRTLVYGLT |
| | | LSPTEEYYKLGAYYHITDIQRRLLK |
| | | AFDALED |
| Q295W + INS45 | 38 | MSGAADVERVYAAMEEAAGLLGVTC |
| on | | AREKIYPLLTEFQDTLTDGGSVVVF |
| (scaffold) | | SMASGRRSTELDFSISVPTSQGDPY |
| SEQ ID NO: 16 | | ATVVDKGLFPATGHPVDDLLADTQK |
| | | HLPVSMFAIDGEVTGGFKKTYAFFP |
| | | TDDMPGVAQLSAIPSMPSSVAENAE |
| | | LFARYGLDKVQMTSMDYKKRQVNLY |
| | | FSELSEQTLAPESVLALVRELGLHV |
| | | PTELGLEFCKRSFSVYPTLNWDTGK |
| | | IDRLCFAVISTDPTLVPSTDERDIE |
| | | QFRHYGTKAPYAYVGENRTLVYGLT |
| | | LSPTEEYYKLGAYYHITDIWRRLLK |
| | | AFDALED |
| Q159S + S212H | 39 | MSGAADVERVYAAMEEAAGLLGVTC |
| Y286V on | | AREKIYPLLTEFQDTLTDGVVVFSM |
| (scaffold) | | ASGRRSTELDFSISVPTSQGDPYAT |
| SEQ ID NO: 16 | | VVDKGLFPATGHPVDDLLADTQKHL |
| | | PVSMFAIDGEVTGGFKKTYAFFPTD |
| | | DMPGVAQLSAIPSMPSSVAENAELF |
| | | ARYGLDKVSMTSMDYKKRQVNLYFS |
| | | ELSEQTLAPESVLALVRELGLHVPT |
| | | ELGLEFCKRSFHVYPTLNWDTGKID |
| | | RLCFAVISTDPTLVPSTDERDIEQF |
| | | RHYGTKAPYAYVGENRTLVYGLTLS |
| | | PTEEYYKLGAVYHITDIQRRLLKAF |
| | | DALED |
| Q161S+ S214H | 40 | MSGAADVERVYAAMEEAAGLLGVTC |
| Y288V + INS45 | | AREKIYPLLTEFQDTLTDGGSVVVF |
| on | | SMASGRRSTELDFSISVPTSQGDPY |
| (scaffold) | | ATVVDKGLFPATGHPVDDLLADTQK |
| SEQ ID NO: 16 | | HLPVSMFAIDGEVTGGFKKTYAFFP |
| | | TDDMPGVAQLSAIPSMPSSVAENAE |
| | | LFARYGLDKVSMTSMDYKKRQVNLY |
| | | FSELSEQTLAPESVLALVRELGLHV |
| | | PTELGLEFCKRSFHVYPTLNWDTGK |
| | | IDRLCFAVISTDPTLVPSTDERDIE |
| | | QFRHYGTKAPYAYVGENRTLVYGLT |
| | | LSPTEEYYKLGAVYHITDIQRRLLK |
| | | AFDALED |
| Q161H + Q295W | 41 | MSGAADVERVYAAMEEAAGLLGVTC |
| + INS45 on | | AREKIYPLLTEFQDTLTDGGSVVVF |
| (scaffold) | | SMASGRRSTELDFSISVPTSQGDPY |
| SEQ ID NO: 23 | | ATVVEKGLFPATGHPVDDLLADTQK |
| | | HLPVSMFAIDGEVTGGFKKTYAFFP |
| | | TDDMPGVAQLSAIPSMPSSVAENAE |
| | | LFARYGLDKVHMTSMDYKKRQVNLY |
| | | FSELSEQTLAPESVLALVRELGLHV |
| | | PTELGLEFCKRSFSVYPTLNWDTGK |
| | | IDRLCFAVISTDPTLVPSTDERDIE |
| | | QFRAYGTKAPYAYVGEKRTLVYGLT |
| | | LSPTEEYYKLGAYYHITDIWRRLLK |
| | | AFDALED |
| Q161S + Q295W | 42 | MSGAADVERVYAAMEEAAGLLGVTC |
| INS45 on | | AREKIYPLLTEFQDTLTDGGSVVVF |
| (scaffold) | | SMASGRRSTELDFSISVPTSQGDPY |
| SEQ ID NO: 23 | | ATVVEKGLFPATGHPVDDLLADTQK |
| | | HLPVSMFAIDGEVTGGFKKTYAFFP |
| | | TDDMPGVAQLSAIPSMPSSVAENAE |
| | | LFARYGLDKVSMTSMDYKKRQVNLY |
| | | FSELSEQTLAPESVLALVRELGLHV |
| | | PTELGLEFCKRSFSVYPTLNWDTGK |
| | | IDRLCFAVISTDPTLVPSTDERDIE |
| | | QFRAYGTKAPYAYVGEKRTLVYGLT |
| | | LSPTEEYYKLGAYYHITDIWRRLLK |
| | | AFDALED |
| Q159S + S212H | 43 | MSGAADVERVYAAMEEAAGLLGVTC |
| on | | AREKIYPLLTEFQDTLTDGVVVFSM |
| (scaffold) | | ASGRRSTELDFSISVPTSQGDPYAT |
| SEQ ID NO: 23 | | VVEKGLFPATGHPVDDLLADTQKHL |
| | | PVSMFAIDGEVTGGFKKTYAFFPTD |
| | | DMPGVAQLSAIPSMPSSVAENAELF |
| | | ARYGLDKVSMTSMDYKKRQVNLYFS |
| | | ELSEQTLAPESVLALVRELGLHVPT |
| | | ELGLEFCKRSFHVYPTLNWDTGKID |
| | | RLCFAVISTDPTLVPSTDERDIEQF |
| | | RAYGTKAPYAYVGEKRTLVYGLTLS |
| | | PTEEYYKLGAYYHITDIQRRLLKAF |
| | | DALED |
| Q159S + Y286V | 44 | MSGAADVERVYAAMEEAAGLLGVTC |
| on | | AREKIYPLLTEFQDTLTDGVVVFSM |
| (scaffold) | | ASGRRSTELDFSISVPTSQGDPYAT |
| SEQ ID NO: 23 | | VVEKGLFPATGHPVDDLLADTQKHL |
| | | PVSMFAIDGEVTGGFKKTYAFFPTD |
| | | DMPGVAQLSAIPSMPSSVAENAELF |
| | | ARYGLDKVSMTSMDYKKRQVNLYFS |
| | | ELSEQTLAPESVLALVRELGLHVPT |
| | | ELGLEFCKRSFSVYPTLNWDTGKID |
| | | RLCFAVISTDPTLVPSTDERDIEQF |
| | | RAYGTKAPYAYVGEKRTLVYGLTLS |
| | | PTEEYYKLGAVYHITDIQRRLLKAF |
| | | DALED |
| S212H + Y286V | 45 | MSGAADVERVYAAMEEAAGLLGVTC |
| on | | AREKIYPLLTEFQDTLTDGVVVFSM |
| (scaffold) | | ASGRRSTELDFSISVPTSQGDPYAT |
| SEQ ID NO: 23 | | VVEKGLFPATGHPVDDLLADTQKHL |
| | | PVSMFAIDGEVTGGFKKTYAFFPTD |
| | | DMPGVAQLSAIPSMPSSVAENAELF |
| | | ARYGLDKVQMTSMDYKKRQVNLYFS |
| | | ELSEQTLAPESVLALVRELGLHVPT |
| | | ELGLEFCKRSFHVYPTLNWDTGKID |
| | | RLCFAVISTDPTLVPSTDERDIEQF |
| | | RAYGTKAPYAYVGEKRTLVYGLTLS |
| | | PTEEYYKLGAVYHITDIQRRLLKAF |
| | | DALED |
| Q161S + INS45 | 46 | MSGAADVERVYAAMEEAAGLLGVTC |
| on | | AREKIYPLLTEFQDTLTDGGSVVVF |
| (scaffold) | | SMASGRRSTELDFSISVPTSQGDPY |
| SEQ ID NO: 23 | | ATVVEKGLFPATGHPVDDLLADTQK |
| | | HLPVSMFAIDGEVTGGFKKTYAFFP |
| | | TDDMPGVAQLSAIPSMPSSVAENAE |
| | | LFARYGLDKVSMTSMDYKKRQVNLY |
| | | FSELSEQTLAPESVLALVRELGLHV |
| | | PTELGLEFCKRSFSVYPTLNWDTGK |
| | | IDRLCFAVISTDPTLVPSTDERDIE |
| | | QFRAYGTKAPYAYVGEKRTLVYGLT |
| | | LSPTEEYYKLGAYYHITDIQRRLLK |
| | | AFDALED |
| S214H + INS45 | 47 | MSGAADVERVYAAMEEAAGLLGVTC |
| on | | AREKIYPLLTEFQDTLTDGGSVVVF |
| (scaffold) | | SMASGRRSTELDFSISVPTSQGDPY |
| SEQ ID NO: 23 | | ATVVEKGLFPATGHPVDDLLADTQK |
| | | HLPVSMFAIDGEVTGGFKKTYAFFP |
| | | TDDMPGVAQLSAIPSMPSSVAENAE |
| | | LFARYGLDKVQMTSMDYKKRQVNLY |
| | | FSELSEQTLAPESVLALVRELGLHV |
| | | PTELGLEFCKRSFHVYPTLNWDTGK |
| | | IDRLCFAVISTDPTLVPSTDERDIE |
| | | QFRAYGTKAPYAYVGEKRTLVYGLT |
| | | LSPTEEYYKLGAYYHITDIQRRLLK |
| | | AFDALED |
| Y288V + INS45 | 48 | MSGAADVERVYAAMEEAAGLLGVTC |
| on | | AREKIYPLLTEFQDTLTDGGSVVVF |
| (scaffold) | | SMASGRRSTELDFSISVPTSQGDPY |
| SEQ ID NO: 23 | | ATVVEKGLFPATGHPVDDLLADTQK |
| | | HLPVSMFAIDGEVTGGFKKTYAFFP |
| | | TDDMPGVAQLSAIPSMPSSVAENAE |
| | | LFARYGLDKVQMTSMDYKKRQVNLY |
| | | FSELSEQTLAPESVLALVRELGLHV |
| | | PTELGLEFCKRSFSVYPTLNWDTGK |
| | | IDRLCFAVISTDPTLVPSTDERDIE |
| | | QFRAYGTKAPYAYVGEKRTLVYGLT |
| | | LSPTEEYYKLGAVYHITDIQRRLLK |
| | | AFDALED |
| Q159H + Q293W | 49 | MSGAADVERVYAAMEEAAGLLGVTC |
| on | | AREKIYPLLTEFQDTLTDGVVVFSM |
| (scaffold) | | ASGRRSTELDFSISVPTSQGDPYAT |
| SEQ ID NO: 23 | | VVEKGLFPATGHPVDDLLADTQKHL |
| | | PVSMFAIDGEVTGGFKKTYAFFPTD |
| | | DMPGVAQLSAIPSMPSSVAENAELF |
| | | ARYGLDKVHMTSMDYKKRQVNLYFS |
| | | ELSEQTLAPESVLALVRELGLHVPT |
| | | ELGLEFCKRSFSVYPTLNWDTGKID |
| | | RLCFAVISTDPTLVPSTDERDIEQF |
| | | RAYGTKAPYAYVGEKRTLVYGLTLS |
| | | PTEEYYKLGAYYHITDIWRRLLKAF |
| | | DALED |
| Q161H + INS45 | 50 | MSGAADVERVYAAMEEAAGLLGVTC |
| on | | AREKIYPLLTEFQDTLTDGGSVVVF |
| (scaffold) | | SMASGRRSTELDFSISVPTSQGDPY |
| SEQ ID NO: 23 | | ATVVEKGLFPATGHPVDDLLADTQK |
| | | HLPVSMFAIDGEVTGGFKKTYAFFP |
| | | TDDMPGVAQLSAIPSMPSSVAENAE |
| | | LFARYGLDKVHMTSMDYKKRQVNLY |
| | | FSELSEQTLAPESVLALVRELGLHV |
| | | PTELGLEFCKRSFSVYPTLNWDTGK |
| | | IDRLCFAVISTDPTLVPSTDERDIE |
| | | QFRAYGTKAPYAYVGEKRTLVYGLT |
| | | LSPTEEYYKLGAYYHITDIQRRLLK |
| | | AFDALED |
| Q295W + INS45 | 51 | MSGAADVERVYAAMEEAAGLLGVTC |
| on | | AREKIYPLLTEFQDTLTDGGSVVVF |
| (scaffold) | | SMASGRRSTELDFSISVPTSQGDPY |
| SEQ ID NO: 23 | | ATVVEKGLFPATGHPVDDLLADTQK |
| | | HLPVSMFAIDGEVTGGFKKTYAFFP |
| | | TDDMPGVAQLSAIPSMPSSVAENAE |
| | | LFARYGLDKVQMTSMDYKKRQVNLY |
| | | FSELSEQTLAPESVLALVRELGLHV |
| | | PTELGLEFCKRSFSVYPTLNWDTGK |
| | | IDRLCFAVISTDPTLVPSTDERDIE |
| | | QFRAYGTKAPYAYVGEKRTLVYGLT |
| | | LSPTEEYYKLGAYYHITDIWRRLLK |
| | | AFDALED |
| Q159S + S212H | 52 | MSGAADVERVYAAMEEAAGLLGVTC |
| Y286V on | | AREKIYPLLTEFQDTLTDGVVVFSM |
| (scaffold) | | ASGRRSTELDFSISVPTSQGDPYAT |
| SEQ ID NO: 23 | | VVEKGLFPATGHPVDDLLADTQKHL |
| | | PVSMFAIDGEVTGGFKKTYAFFPTD |
| | | DMPGVAQLSAIPSMPSSVAENAELF |
| | | ARYGLDKVSMTSMDYKKRQVNLYFS |
| | | ELSEQTLAPESVLALVRELGLHVPT |
| | | ELGLEFCKRSFHVYPTLNWDTGKID |
| | | RLCFAVISTDPTLVPSTDERDIEQF |
| | | RAYGTKAPYAYVGEKRTLVYGLTLS |
| | | PTEEYYKLGAVYHITDIQRRLLKAF |
| | | DALED |
| Q161S + S214H | 53 | MSGAADVERVYAAMEEAAGLLGVTC |
| Y288V + INS45 | | AREKIYPLLTEFQDTLTDGGSVVVF |
| on | | SMASGRRSTELDFSISVPTSQGDPY |
| (scaffold) | | ATVVEKGLFPATGHPVDDLLADTQK |
| SEQ ID NO: 23 | | HLPVSMFAIDGEVTGGFKKTYAFFP |
| | | TDDMPGVAQLSAIPSMPSSVAENAE |
| | | LFARYGLDKVSMTSMDYKKRQVNLY |
| | | FSELSEQTLAPESVLALVRELGLHV |
| | | PTELGLEFCKRSFHVYPTLNWDTGK |
| | | IDRLCFAVISTDPTLVPSTDERDIE |
| | | QFRAYGTKAPYAYVGEKRTLVYGLT |
| | | LSPTEEYYKLGAVYHITDIQRRLLK |
| | | AFDALED |
| INS45 | 54 | MSGAADVERVYAAMEEAAGLLGVTC |
| (GS insertion) | | AREKIYPLLTEFQDTLTDGGSVVVF |
| on | | SMASGRRSTELDFSISVPTSQGDPY |
| Scaffold | | ATVVDKGLFPATGHPVDDLLADTQK |
| SEQ ID NO: 16 | | HLPVSMFAIDGEVTGGFKKTYAFFP |
| | | TDDMPGVAQLSAIPSMPSSVAENAE |
| | | LFARYGLDKVQMTSMDYKKRQVNLY |
| | | FSELSEQTLAPESVLALVRELGLHV |
| | | PTELGLEFCKRSFSVYPTLNWDTGK |
| | | IDRLCFAVISTDPTLVPSTDERDIE |
| | | QFRHYGTKAPYAYVGENRTLVYGLT |
| | | LSPTEEYYKLGAYYHITDIQRRLLK |
| | | AFDALED |
| INS45 | 55 | MSGAADVERVYAAMEEAAGLLGVTC |
| (GS insertion) | | AREKIYPLLTEFQDTLTDGGSVVVF |
| on | | SMASGRRSTELDFSISVPTSQGDPY |
| Scaffold | | ATVVEKGLFPATGHPVDDLLADTQK |
| SEQ ID NO: 23 | | HLPVSMFAIDGEVTGGFKKTYAFFP |
| | | TDDMPGVAQLSAIPSMPSSVAENAE |
| | | LFARYGLDKVQMTSMDYKKRQVNLY |
| | | FSELSEQTLAPESVLALVRELGLHV |
| | | PTELGLEFCKRSFSVYPTLNWDTGK |
| | | IDRLCFAVISTDPTLVPSTDERDIE |
| | | QFRAYGTKAPYAYVGEKRTLVYGLT |
| | | LSPTEEYYKLGAYYHITDIQRRLLK |
| | | AFDALED |

In some aspects, a prenyltransferase template into which the one or more variations (also referred to herein as “mutation” or “substitution”) are introduced to create a variant is a prenyltransferase sequence having 35% or greater identity, 50% or greater identity, 60% or greater identity, 65% or greater identity, 70% or greater identity, 75% or greater identity, 80% or greater identity, 85% or greater identity, 87.5% or greater identity, 90% or greater identity, 92.5% or greater identity, or 95% or greater identity, to SEQ ID NO: 1. In other aspects, the prenyltransferase template is any one of SEQ ID NOs: 2-27, and preferably SEQ ID NO: 16 or SEQ ID NO: 15. Variants of the prenyltransferase template SEQ ID NO: 16 preferably include at least (i) Q159H; and (ii) Q293W, Q293H, Q293C, Q293A, Q293S, Q293V, Q293D, Q293Y, or Q293E mutations as described herein, or even more preferably (i) Q159H; and (ii) Q293W mutations as described herein.

One, or more than one, amino acid variation can be described relative to the location of a particular amino acid in a wild type prenyltransferase template sequence. Identification of locations in the template that when substituted with variant amino acids which provide desired activity and regioselectivity can be determined by testing methods as described herein. For example, in the prenyltransferase template SEQ ID NO: 1, one or more of the following positions may be subject to substitution with an amino acid that is different than the wild type amino acid at that location: A17, C25, Q38, V49, S51, A53, M106, A108, E112, K118, K119, Y121, F123, T126, Q161, M162, D166, N173, L174, S177, G205, C209, F213, S214, Y216, L219, D227, 8228, C230, A232, 1234, T269, L270, V271, L274, Y283, G286, A287, Y288, V294, Q295, L298, and F302K.

However, in other prenyltransferase templates, the location of the target amino acid for substitution may be different but corresponds to the orthologous positions identified for SEQ ID NO: 1, which is the reference template herein. For example, in a prenyltransferase sequence that is different than SEQ ID NO: 1, the target orthologous amino acids can be shifted by an “indel” in the range of 10 to −1, or in the range of +1 to +10, based on the particular amino acid variation location. Note that “indel” events for other homologs could be in a range that is of an absolute size that is greater than 10 amino acids, although the current alignment of FIG. 5 contains indels of absolute size that are smaller than 10. For example, using the alignment of FIG. 5 as a guide, amino acid position 161 of SEQ ID NO: 1 corresponds to position 159 in SEQ ID NO: 16, and likewise amino acid position 295 of SEQ ID NO: 1 corresponds to position 293 in SEQ ID NO: 16. In some cases, the shift can vary along the length of the sequence that is aligned to SEQ ID NO: 1. For example, the shift may increase or decrease after a first stretch of amino acids in the aligned sequence, and then may increase or decrease after a second stretch of amino acids in the aligned sequence, etc. The shift of shifts can be determined by the gaps between the template and aligned sequence along the length of the proteins.

Art known methods can be used for the testing the enzymatic activity of prenyltransferase, and such methods can be used to test activity of prenyltransferase variant enzymes as well. As a general matter, an in vitro reaction composition including a prenyltransferase variant (purified or in cell lysate or cell extract), geranyl pyrophosphate and olivetolic acid (substrates) can convert the substrates to the product geranyl-olivetolate (e.g., GOLA). Of particular interest herein is conversion of geranyl pyrophosphate and olivetolic acid to CBGA.

In some aspects, non-natural prenyltransferases with one or more variant amino acids as described herein are enzymatically capable of at least two-fold, at least three-fold, at least four-fold, at least five-fold, at least six-fold, at least seven-fold, at least eight-fold, at least nine-fold, at least ten-fold, at least eleven-fold, at least twelve-fold, at least thirteen-fold, at least fourteen-fold, at least fifteen-fold, at least sixteen-fold, at least seventeen-fold, at least eighteen-fold, at least-nineteen fold, or at least-twenty fold greater rate of formation of cannabigerolic acid from geranyl pyrophosphate and olivetolic acid, as compared to the wild type prenyltransferase. Variants were also identified that displayed very high activity on the order of about 300 fold or greater rate of formation of cannabigerolic acid from geranyl pyrophosphate and olivetolic acid, as compared to the wild type prenyltransferase. For example, the increase in rate of formation of cannabigerolic acid from geranyl pyrophosphate and olivetolic acid, as compared to the wild type prenyltransferase, can be in the range of about 2× to about 300×, about 5× to about 300×, or about 10× to about 300× as determined in an in vitro enzymatic reaction using purified prenyltransferase variant.

Non-natural prenyltransferases with one or more variant amino acids of the disclosure can be enzymatically capable of at least two-fold, at least three-fold, at least four-fold, at least five-fold, at least six-fold, at least seven-fold, at least eight-fold, at least nine-fold, at least ten-fold, at least eleven-fold, at least twelve-fold, at least-thirteen fold, at least fourteen-fold, at least fifteen-fold, at least sixteen-fold, at least seventeen-fold, at least eighteen-fold, at least nineteen-fold, or at least-twenty fold greater rate of formation of cannabigerovarinic acid (CBGVA) from geranyl pyrophosphate (GPP) and divarinolic acid (DVA), or of cannabigerorcinic acid (CBGOA) from geranyl pyrophosphate (GPP) and orsellinic acid (OSA), as compared to the wild type prenyltransferase. For example, the increase in rate of formation of CBGVA from GPP and DVA as compared to the wild type prenyltransferase, can be in the range of about 2× to about 450×, about 5× to about 400×, or about 10× to about 375×. The increase in rate of formation of CBGOA from GPP and OSA as compared to the wild type prenyltransferase, can be in the range of about 2× to about 600×, about 5× to about 575×, or about 10× to about 550×.

Non-natural prenyltransferases with one or more variant amino acids of the disclosure can be enzymatically capable of at least two-fold, at least three-fold, at least four-fold, at least five-fold, at least six-fold, at least seven-fold, at least eight-fold, at least nine-fold, at least ten-fold, at least eleven-fold, at least twelve-fold, at least-thirteen fold, at least fourteen-fold, at least fifteen-fold, at least sixteen-fold, at least seventeen-fold, at least eighteen-fold, at least nineteen-fold, or at least-twenty fold greater rate of formation of cannabigerovarinic acid (CBGVA) from geranyl pyrophosphate (GPP) and divarinolic acid (DVA), or of cannabigerorcinic acid (CBGOA) from geranyl pyrophosphate (GPP) and orsellinic acid (OSA), as compared to the wild type prenyltransferase. For example, the increase in rate of formation of CBGVA from GPP and DVA as compared to the wild type prenyltransferase, can be in the range of about 2× to about 450×, about 5× to about 400×, or about 10× to about 375×. The increase in rate of formation of CBGOA from GPP and OSA as compared to the wild type prenyltransferase, can be in the range of about 2× to about 600×, about 5× to about 575×, or about 10× to about 550×.

Non-natural prenyltransferases with one or more variant amino acids of the disclosure can be enzymatically capable of at least two-fold, at least three-fold, at least four-fold, at least five-fold, at least six-fold, at least seven-fold, at least eight-fold, at least nine-fold, at least ten-fold, at least eleven-fold, at least twelve-fold, at least-thirteen fold, at least fourteen-fold, at least fifteen-fold, at least sixteen-fold, at least seventeen-fold, at least eighteen-fold, at least nineteen-fold, or at least-twenty fold greater rate of formation of a 2-prenylated 5-alkylbenzene-1,3-diol (e.g., CBG; 2-GOL) from geranyl pyrophosphate and 5-alkylbenzene-1,3-diol (e.g., olivetol), as compared to the wild type prenyltransferase. For example, the increase in rate of formation of CBG from GPP and olivetol, as compared to the wild type prenyltransferase, can be in the range of about 2× to about 200×, about 5× to about 175×, or about 10× to about 150×.

Using a purified prenyltransferase preparation the rate of formation of CBGA can be determined. The rate can be expressed in terms of mM CBGA/min/mM enzyme. Reaction conditions can be as follows: each prenylation reaction assay was performed in a volume of 20 microliters and contained 20 millimolar magnesium chloride (MgCl₂), 2 millimolar donor molecule (e.g., GPP), 100 millimolar HEPES buffer at a pH of 7.5, 2 millimolar substrate (e.g., olivetolic acid), and 20 micrograms prenyltransferase protein. These reactions were incubated for 16 hours at 30° C. To assess prenylation activity by HPLC the prenylation products were extracted from the assay reaction with the following protocol: 40 microliters of ethyl acetate was added to each reaction and vortexed thoroughly. After vortexing, each reaction was centrifuged for 10 minutes at 14,000G. The top layer (“organic layer”) was collected. This was repeated twice. The collected organic layer was evaporated, and the resulting residue was resuspended in 40 microliters of 100% methanol. After resuspending in methanol, 40 microliters of 100% HPLC grade water was added to bring the final solution to 50% methanol. These will be referred to as the “variant reactions with GPP”. For analysis of CBGA (3-GOLA) and 5-GOLA, the final 50% methanol solutions were run on a Thermo Fisher UltiMate 3000 UHPLC with an Acclaim RSLC 120 angstrom C18 column with a 4 millimeter Phenomonex Securityguard guard column (54 millimeter total column length).

Likewise, using a purified prenyltransferase variant preparation, the rate of formation of cannabigerovarinic acid (CBGVA) from geranyl pyrophosphate and divarinolic acid (DVA) can be determined using similar methods, as well as the rate of formation of cannabigerorcinic acid (CBGOA) from geranyl pyrophosphate and orsellinic acid (OSA) (see FIG. 7A and FIG. 7B) and the rate of formation of cannabigerol (CBG) from olivetol and geranyl pyrophosphate (see FIG. 8).

In some aspects, the prenyltransferase variants may provide a rate of formation of CBGA of greater than 0.005 mM CBGA/min/mM enzyme, greater than about 0.010 mM CBGA/min/mM enzyme, greater than about 0.020 mM CBGA/min/mM enzyme, greater than about 0.050 mM CBGA/min/mM enzyme, greater than about 0.100 mM CBGA/min/mM enzyme, greater than about 0.250 mM CBGA/min/mM enzyme, greater than about 0.500 mM CBGA/min/mM enzyme, such as in the range of about 0.005 mM or 0.010 mM to about 1.250 mM CBGA/min/mM enzyme, or in the range of about 0.020 mM to about 1.0 mM CBGA/min/mM enzyme.

In some aspects, the prenyltransferase variants may provide a rate of formation of CBGVA from DVA and GPP, of CBGOA from OSA and GPP, or of CBG from olivetol and GPP, according to any of the rates as described herein.

In some aspects, non-natural prenyltransferases with one or more variant amino acids as describe herein may be enzymatically capable of providing regioselectivity to 3-geranyl-olivetolate (CBGA; 3-GOLA). In some aspects, the non-natural prenyltransferases with one or more variant amino acids provide an amount of regioselectivity to 3-geranyl-olivetolate CBGA of 60% or greater, 70% or greater, 80% or greater, 85% or greater, 90% or greater, 91% or greater, 92% or greater, 93% or greater, 94% or greater, 95% or greater, 96% or greater, 97% or greater, 98% or greater, 99% or greater, 99.2% or greater, 99.4% or greater, 99.5% or greater, 99.6% or greater, 99.7% or greater, 99.8% or greater, 99.9% or greater, 99.95% or greater, or 100% regioselectivity to 3-geranyl-olivetolate (CBGA; 3-GOLA) of the total geranyl olivetolate (3-GOLA plus 5-GOLA) as determined in an in vitro enzymatic reaction using purified prenyltransferase variant. Accordingly, of the geranyl-olivetolate reaction products, 5-GOLA may be in an amount of less than 10% (wt), less than 9% (wt), less than 8% (wt), less than 7% (wt), less than 6 (wt), less than 5% (wt), less than 4% (wt), less than 3% (wt), less than 2% (wt), less than 1% (wt), less than 0.8% (wt), less than 0.6% (wt), less than 0.5% (wt), less than 0.4% (wt), less than 0.3% (wt), less than 0.2% (wt), less than 0.1% (wt), less than 0.05% (wt) or 0.0% (wt). In view of the improved regioselectivity of the prenyltransferase variants, the disclosure also provides compositions that are enriched for desired cannabinoids and derivatives thereof. In particular, the disclosure provides compositions enriched for CBGA (3-GOLA) and/or CBG. Enriched compositions include those that are pharmaceutical compositions as well as those that are used for non-pharmaceutical purposes, such as having 90% or greater 3-GOLA as described herein, or other desired derivatives depending on the provided substrate (e.g., olivetol, olivetolic acid, etc.) as described elsewhere herein. In some aspects, non-natural prenyltransferase with one or more variant amino acids as described herein display an increase in rate of formation of cannabigerolic acid from geranyl pyrophosphate and olivetolic acid, in any of the amounts described herein, and regioselectivity in any of the amounts as described herein.

In some aspects, non-natural prenyltransferases with one or more variant amino acids as described herein are enzymatically capable of providing regioselectivity to 3-geranyl-orsellinate (3-GOSA), an isomer of cannabigerorcinic acid (CBGOA) formed after reacting GPP and OSA. In some aspects, the non-natural prenyltransferases with one or more variant amino acids provide an amount of regioselectivity to 3-GOSA of 60% or greater, 70% or greater, 80% or greater, 85% or greater, 90% or greater, 91% or greater, 92% or greater, 93% or greater, 94% or greater, 95% or greater, 96% or greater, 97% or greater, 98% or greater, 99% or greater, 99.2% or greater, 99.4% or greater, 99.5% or greater, 99.6% or greater, 99.7% or greater, 99.8% or greater, 99.9% or greater, 99.95% or greater, or 100% regioselectivity to 3-GOSA of the total geranyl-orsellinate (3-GOSA plus 5-GOSA) as determined in an in vitro enzymatic reaction using purified prenyltransferase variant.

Accordingly, of the geranyl-orsellinate reaction product, 5-GOSA may be in an amount of less than 10% (wt), less than 9% (wt), less than 8% (wt), less than 7% (wt), less than 6 (wt), less than 5% (wt), less than 4% (wt), less than 3% (wt), less than 2% (wt), less than 1% (wt), less than 0.8% (wt), less than 0.6% (wt), less than 0.5% (wt), less than 0.4% (wt), less than 0.3% (wt), less than 0.2% (wt), less than 0.1% (wt), less than 0.05% (wt) or 0.0% (wt). In view of the improved regioselectivity of the prenyltransferase variants, the disclosure also provides compositions that are enriched for 3-GOSA, and derivatives thereof, such as pharmaceutical and non-pharmaceutical compositions having 90% or greater 3-GOSA as described herein, or other desired derivatives thereof.

In some aspects, non-natural prenyltransferases with one or more variant amino acids as describe herein are enzymatically capable of providing regioselectivity to cannabigerol (CBG; 2-GOL) instead of the 4-GOL isomer, formed after reacting olivetol and GPP. In some aspects, the non-natural prenyltransferases with one or more variant amino acids provide an amount of regioselectivity to 2-GOL of 60% or greater, 70% or greater, 80% or greater, 85% or greater, 90% or greater, 91% or greater, 92% or greater, 93% or greater, 94% or greater, 95% or greater, 96% or greater, 97% or greater, 98% or greater, 99% or greater, 99.2% or greater, 99.4% or greater, 99.5% or greater, 99.6% or greater, 99.7% or greater, 99.8% or greater, 99.9% or greater, 99.95% or greater, or 100% regioselectivity to 2-GOL of the total cannabigerol isomers (2-GOL plus 4-GOL) as determined in an in vitro enzymatic reaction using purified prenyltransferase variant.

Accordingly, of the GPP—olivetol reaction product, 4-GOL may be in an amount of less than 10% (wt), less than 9% (wt), less than 8% (wt), less than 7% (wt), less than 6 (wt), less than 5% (wt), less than 4% (wt), less than 3% (wt), less than 2% (wt), less than 1% (wt), less than 0.8% (wt), less than 0.6% (wt), less than 0.5% (wt), less than 0.4% (wt), less than 0.3% (wt), less than 0.2% (wt), less than 0.1% (wt), less than 0.05% (wt) or 0.0% (wt). In view of the improved regioselectivity of the prenyltransferase variants, the disclosure also provides compositions that are enriched for 2-GOL, and derivatives thereof, such as pharmaceutical and non-pharmaceutical compositions having 90% or greater 2-GOL as described herein, or other desired derivatives thereof.

The non-natural prenyltransferases of the disclosure can include one amino acid variation, two amino acid variations, three amino acid variations, four amino acid variations, five amino acid variations, or more than five amino acid variations, from a wild type prenyltransferase template sequence. The variation(s) can be any single or combinations as described herein. Optional variations, other than those described herein, can be used with any single or combinations as described herein, wherein the optional variations are not detrimental to the desired activity of the prenyltransferase variants. Exemplary optional variations include those such as conservative amino acid substitutions that do not considerably alter protein properties.

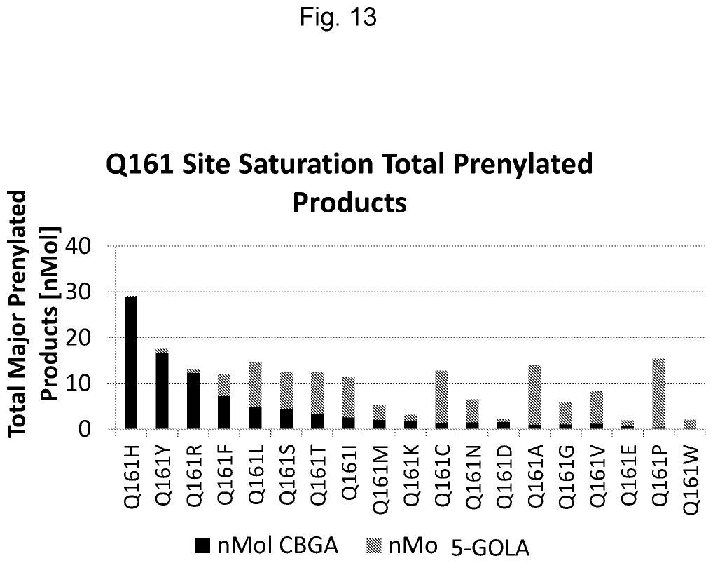

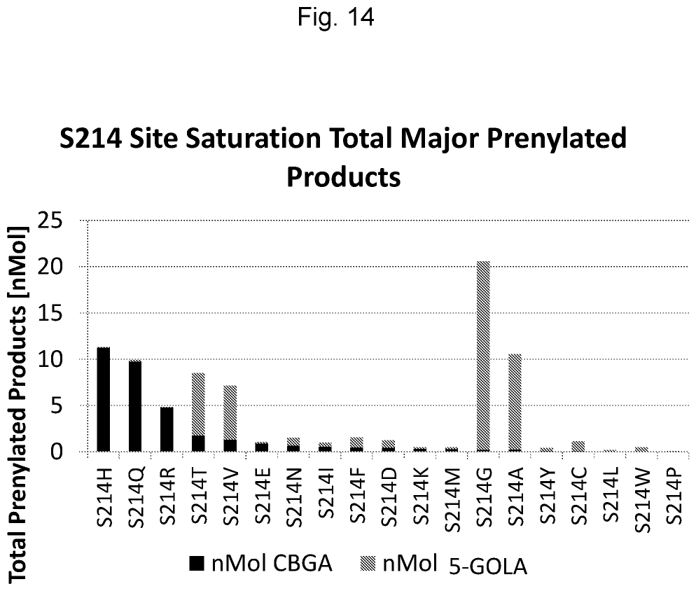

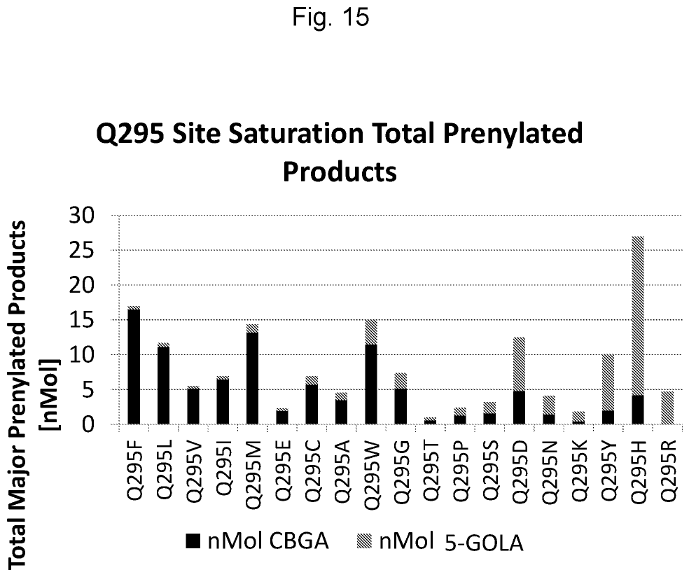

FIGS. 13-15 exhibit performance of variants of amino acid site saturation for Q161, S214, and Q295 with the prenyltransferase SEQ ID NO: 1 used as a template. These results present some positions of amino acids mutations providing (a) enzymatic activity of at least two-fold greater rate of formation of cannabigerolic acid (CBGA) from geranyl pyrophosphate and olivetolic acid, as compared to the wild type prenyltransferase or; (b) 50% or greater regioselectivity to CBGA (3-GOLA); or both (a) and (b). The mutations are described with reference to the numbering of amino acid positions in SEQ ID NO: 1 (NphB); however, one or more of the mutations can be introduced into SEQ ID NO: 16 at the respective orthologous positions or into other prenyltransferase homologs at corresponding orthologous amino acid positions to provide variants with desired activity and/or regioselectivity. The alignments shown in FIG. 5, or alignments of any other soluble prenyltransferase sequence with SEQ ID NO: 1 (NphB), can be used as a guide for introducing one or more variations into a desired template sequence at orthologous positions.

Results of the mutagenesis procedures revealed a number of amino acid variants along the prenyltransferase template showing at least two-fold greater rate of formation of cannabigerolic acid (CBGA) from geranyl pyrophosphate and olivetolic acid, as compared to the wild type prenyltransferase; or (b) 50% or greater regioselectivity to CBGA (3-GOLA); or both (a) and (b).

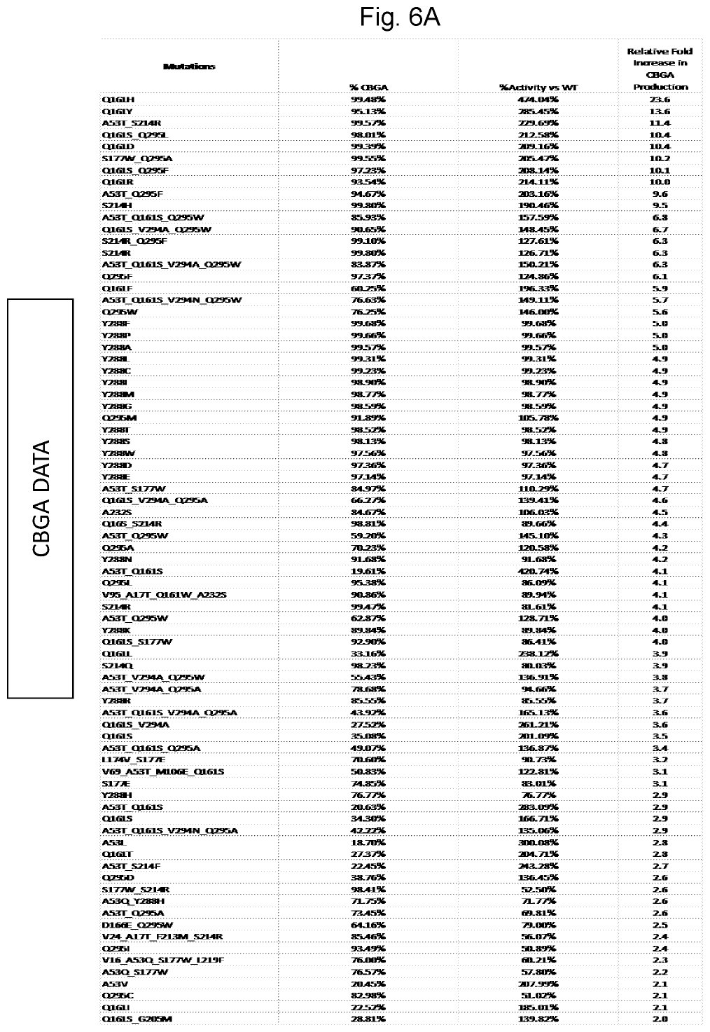

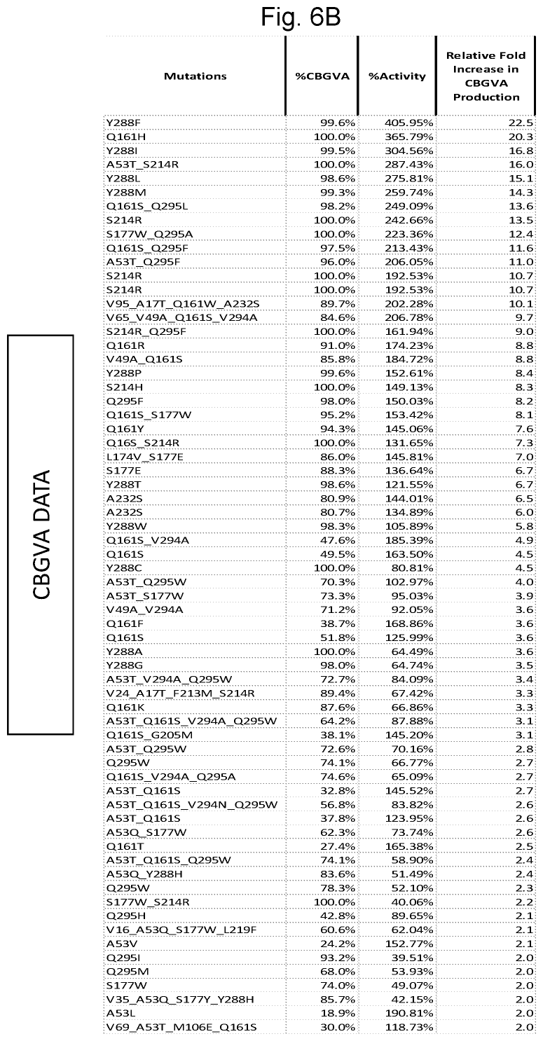

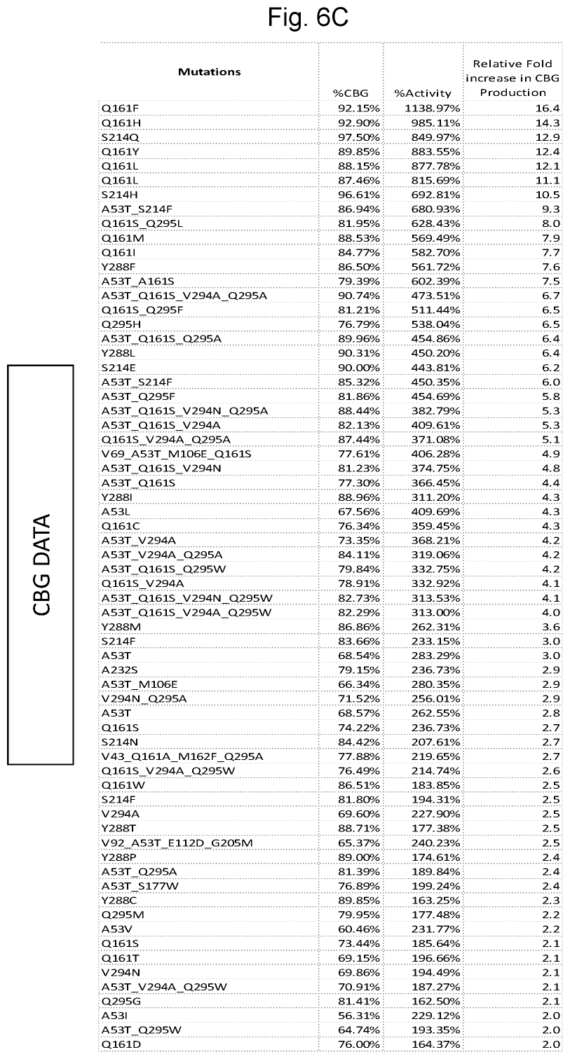

FIGS. 6A-6C list positions of amino acids mutations and substitution combinations providing (a) enzymatic activity of at least two-fold greater rate of formation of cannabigerolic acid (CBGA) from geranyl pyrophosphate (GPP) and olivetolic acid; (b) cannabigerovarinic acid (3-GDVA) from GPP and divarinolic acid (DVA); and (c) CBG (2-GOL) from olivetol and GPP, as compared to the wild type prenyltransferase. The data in FIGS. 6A-6C also reflect the regioselectivity to CDBA (3-GOLA), 3-GDVA, and CBG (2-GOL). The mutations are described with reference to the numbering of amino acid positions in SEQ ID NO: 1 (NphB). The variant location and identities as set forth in this table, used in combination with the alignments shown in FIG. 5, can be used to introduce the variant amino acids into any other soluble prenyltransferase sequence that can be aligned with SEQ ID NO: 1.

In some aspects, the non-natural prenyltransferase is based on reference template SEQ ID NO: 1 (NphB) or has 35% or greater identity, 50% or greater identity, 60% or greater identity, 65% or greater identity, 70% or greater identity, 75% or greater identity, 80% or greater identity, 85% or greater identity, 87.5% or greater identity, 90% or greater identity, 92.5% or greater identity, or 95% or greater identity, to SEQ ID NO: 1 and has one or more amino acid variations at position(s) selected from the group consisting of: A17, C25, Q38, V49, S51, A53, M106, A108, E112, K118, K119, Y121, F123, T126, Q161, M162, D166, N173, L174, S177, G205, C209, F213, S214, Y216, L219, D227, R228, C230, A232, 1234, T269, L270, V271, L274, Y283, G286, A287, Y288, V294, Q295, L298, and F302K, with reference to the amino acid sequence of SEQ ID NO: 1. As noted previously, positions recited herein are with reference to the amino acid sequence of SEQ ID NO: 1, even if not expressly recited as such.

In other aspects, the prenyltransferase template is any one of SEQ ID NOs: 2-27, or a homolog thereof, that include these variations at the corresponding positions. For example, in SEQ ID NO: 16, the variant positions are shifted −2 from these locations, and therefore SEQ ID NO: 16 can have one or more amino acid variations at position(s) selected from the group consisting of: A17, C25, Q38, V47, S49, A51, M104, A106, E110, K116N, K117, Y119, F121, T124, Q159, M160, D164, N171, L172, S175, G203, C207, F211, S212, Y214, L217, D225, R226, C228, A230, 1232, T267, L268, V269, L272, Y281, G284, A285, Y286, 1292, Q293, L296, and F300, with reference to the orthologous amino acid positions of SEQ ID NO: 1. As another example, in SEQ ID NO: 15, the variant positions are shifted by different amounts along the length of the protein (e.g., 0 (first stretch), +1 (second stretch), +2 (third stretch), +8 (fourth stretch), and +4 (fifth stretch)). As based on the alignment, SEQ ID NO: 15 can have one or more amino acid variations at position(s) selected from the group consisting of: S17, C25, Q38, V47, S49, A51, G105, A107, G111, K117, K118, Y120, F122, Q125, T161, M162, D166, N173, L174, G177, M205, A209, F213, A214, Y216, L219, E227, R228, C230, A232, 1240, M271, L272, V273, V276, Y285, G288, S289, Y290, Q296, T297, L300, and F304, with reference to the orthologous amino acid positions of SEQ ID NO: 1.

In some aspects, the non-natural prenyltransferase is based on reference template SEQ ID NO: 16 or has 50% or greater identity, 60% or greater identity, 65% or greater identity, 70% or greater identity, 75% or greater identity, 80% or greater identity, 85% or greater identity, 87.5% or greater identity, 90% or greater identity, 92.5% or greater identity, or 95% or greater identity, to SEQ ID NO: 16 and has one or more amino acid variations at position(s) selected from the group consisting of: A17T, C25V, Q38G, V47A, V47L, V47S, S49T, A51C, A51D, A51E, A51F, A51G, A51H, A51I, A51K, A51L, A51M, A51N, A51P, A51Q, A51R, A51S, A51T, A51V, A51W, A51Y, M104E, A106G, E110D, E110G, K116N, K116Q, K117A, K117D, Y119W, F121L, F121A, F121H, F121W, T124R, Q159H, Q159R, Q159S, Q159T, Q159Y, Q159A, Q159F, Q159G, Q159I, Q159K, Q159L, Q159M, Q159C, Q159D, Q159E, Q159N, Q159P, Q159V, Q159W, M160A, M160F, D164E, N171D, L172V, S175E, S175W, S175Y, S175H, S175K, S175R, G203L, G203M, C207G, F211M, S212A, S212C, S212D, S212E, S212F, S212G, S212I, S212K, S212L, S212M, S212N, S212P, S212Q, S212R, S212T, S212V, S212W, S212Y, S212H, Y214A, L217F, D225E, R226E, R226Q, C228N, C228S, A230S, I232H, T267W, L268Y, V269E, L272V, Y281L, G284E, A285Y, Y286A, Y286F, Y286L, Y286M, Y286P, Y286T, Y286V, Y286C, Y286D, Y286E, Y286G, Y286H, Y286I, Y286K, Y286N, Y286Q, Y286R, Y286S, Y286W, I292A, I292F, I292N, Q293G, Q293K, Q293L, Q293N, Q293P, Q293R, Q293F, Q293W, Q293H, Q293C, Q293A, Q293S, Q293V, Q293D Q293Y, Q293E, Q293I, Q293M, Q293T, L296A, L296Q, L296W, and F300K.

In other aspects, the prenyltransferase template is any one of SEQ ID NOs: 2-27, or a homolog thereof, that include these variations at the corresponding orthologous positions as determined in the MSA of FIG. 5. For example, in SEQ ID NO: 1 (NphB) the variant positions are shifted +2, from the locations in SEQ ID NO: 16. As such, SEQ ID NO: 1 (NphB) or a sequence having different degree of identity to SEQ ID NO: 1 (NphB) (e.g., 50% or greater or up to 95% or greater as discussed herein) has one or more amino acid variations at position(s) selected from the group consisting of: A17T, C25V, Q38G, V49A, V49L, V49S, S51T, A53C, A53D, A53E, A53F, A53G, A53H, A53I, A53K, A53L, A53M, A53N, A53P, A53Q, A53R, A53S, A53T, A53V, A53W, A53Y, M106E, A108G, E112D, E112G, K118N, K118Q, K119A, K119D, Y121W, F123L, F123A, F123H, F123W, T126R, Q161H, Q161R, Q161S, Q161T, Q161Y, Q161A, Q161F, Q161G, Q161I, Q161K, Q161L, Q161M, Q161C, Q161D, Q161E, Q161N, Q161P, Q161V, Q161W, M162A, M162F, D166E, N173D, L174V, S177E, S177W, S177Y, S177H, S177K, S177R, G205L, G205M, C209G, F213M, S214A, S214C, S214D, S214E, S214F, S214G, S214I, S214K, S214L, S214M, S214N, S214P, S214Q, S214R, S214T, S214V, S214W, S214Y, S214H, Y216A, L219F, D227E, R228E, R228Q, C230N, C230S, A232S, I234H, T269W, L270Y, V271E, L274V, Y283L, G286E, A287Y, Y288A, Y288F, Y288L, Y288M, Y288P, Y288T, Y288V, Y288C, Y288D, Y288E, Y288G, Y288H, Y288I, Y288K, Y288N, Y288Q, Y288R, Y288S, Y288W, V294A, V294F, V294N, Q295G, Q295K, Q295L, Q295N, Q295P, Q295R, Q295F, Q295W, Q295H, Q295C, Q295A, Q295S, Q295V, Q295D, Q295Y, Q295E, Q295I, Q295M, Q295T, L298A, L298Q, L298W, and F302K.

Accordingly, expressly contemplated for each template herein, the non-natural prenyltransferase is based on any one of templates SEQ ID NOs: 2-27, or has 50% or greater identity, 60% or greater identity, 65% or greater identity, 70% or greater identity, 75% or greater identity, 80% or greater identity, 85% or greater identity, 87.5% or greater identity, 90% or greater identity, 92.5% or greater identity, or 95% or greater identity, to any one of SEQ ID NOs: 2-27, and has one or more amino acid variations at orthologous position(s) selected from the group consisting of those positions corresponding to those listed for SEQ ID NO: 1 (NphB), as determined in the MSA in FIG. 5. In a similar fashion, these orthologous positions may be determined by multiple sequence alignment for other homologs not presented here. These orthologous positions listed for SEQ ID NO:1 are considered novel across all prenyltransferase homologs.

In some aspects, the non-natural prenyltransferase is based on template SEQ ID NO: 16 or has 50% or greater identity, 60% or greater identity, 65% or greater identity, 70% or greater identity, 75% or greater identity, 80% or greater identity, 85% or greater identity, 87.5% or greater identity, 90% or greater identity, 92.5% or greater identity, or 95% or greater identity, to SEQ ID NO: 16 and has two or more amino acid variations at position(s) selected from, but not limited to, the group consisting of: (i) Q159A and (ii) Q293F, Q293M, Q293F, Q293F; (i) Q159F and (ii) Q293F, Q293W, or Q293H; (i) Q159G and (ii) Q293F; (i) Q159H and (ii) Q293W, Q293H, Q293C, Q293A, Q293S, Q293V, Q293D, Q293Y, or Q293E; (i) Q159I and (ii) Q293F; (i) Q159K and (ii) Q293V or Q293V; (i) Q159L and (ii) Q293W or Q293F; (i) Q159M and (ii) Q293F or Q293W; (i) Q159R and (ii) Q293V, Q293M, or Q293T; (i) Q159S and (ii) Y286I; and (i) S175H and (ii) Q293V.

In other aspects, the prenyltransferase template is any of SEQ ID NOs: 1-15, 17-27, or a homolog thereof, that include these variations at the corresponding positions. For example, in SEQ ID NO: 1 (NphB) the variant positions are shifted +2, compared to the orthologous positions shown above for SEQ ID NO:16. Accordingly, expressly contemplated for each template, the non-natural prenyltransferase is based on any one of the template SEQ ID NOs: 1-15, 17-27 or has 50% or greater identity, 60% or greater identity, 65% or greater identity, 70% or greater identity, 75% or greater identity, 80% or greater identity, 85% or greater identity, 87.5% or greater identity, 90% or greater identity, 92.5% or greater identity, or 95% or greater identity, to the template selected from SEQ ID NOs: 1-15, 17-27, and has two or more amino acid variations at orthologous position(s) selected from, but not limited to, the group consisting of the following based on SEQ ID NO: 16: (i) Q159A and (ii) Q293F, Q293M, Q293F, Q293F; (i) Q159F and (ii) Q293F, Q293W, or Q293H; (i) Q159G and (ii) Q293F; (i) Q159H and (ii) Q293W, Q293H, Q293C, Q293A, Q293S, Q293V, Q293D, Q293Y, or Q293E; (i) Q159I and (ii) Q293F; (i) Q159K and (ii) Q293V or Q293V; (i) Q159L and (ii) Q293W or Q293F; (i) Q159M and (ii) Q293F or Q293W; (i) Q159R and (ii) Q293V, Q293M, or Q293T; (i) Q159S and (ii) Y286I; and (i) S175H and (ii) Q293V.

In some aspects, the non-natural prenyltransferase is based on template SEQ ID NO: 16 or has 50% or greater identity, 60% or greater identity, 65% or greater identity, 70% or greater identity, 75% or greater identity, 80% or greater identity, 85% or greater identity, 87.5% or greater identity, 90% or greater identity, 92.5% or greater identity, or 95% or greater identity, to SEQ ID NO: 16 and has three or more amino acid variations at position(s) selected from, but not limited to, the group consisting of (i) Q159H, (ii) Y286A, and (iii) Q293F, Q293M, or Q293V; (i) Q159H, (ii) Y286I, and (iii) Q293M or Q293V; (i) Q159H, (ii) Y286V, and (iii) Q293F, Q293M, Q293V, or Q293W; (i) Q159L, (ii) S175H, and (iii) Q293F; (i) S175H, (ii), Y286V, and (iii) Q293M; (i) S175H, (ii), Y286I, and (iii) Q293M or Q293V; (i) Q159S, (ii) S175H, and (iii) Y286I; (i) Q159S, (ii) S175R, and (iii) Y286V; (i) Q159S, (ii) S175S, and (iii) Y286I; and (i) Q159S, (ii) S212H, and (iii) Y286A or Y286V.

In other aspects, the prenyltransferase template is any of SEQ ID NOs: 1-15, 17-27, or a homolog thereof, that include these variations at the corresponding positions. For example, in SEQ ID NO: 1 (NphB), the variant positions are shifted +2, compared to the orthologous positions shown above for SEQ ID NO:16. Accordingly, expressly contemplated for each template, the non-natural prenyltransferase is based on any one of the template SEQ ID NOs: 1-15, 17-27 or has 50% or greater identity, 60% or greater identity, 65% or greater identity, 70% or greater identity, 75% or greater identity, 80% or greater identity, 85% or greater identity, 87.5% or greater identity, 90% or greater identity, 92.5% or greater identity, or 95% or greater identity, to the template selected from SEQ ID NOs: 1-15, 17-27, and has three or more amino acid variations at position(s) selected from, but not limited to, the group consisting of the following based on SEQ ID NO: 16: (i) Q159H, (ii) Y286A, and (iii) Q293F, Q293M, or Q293V; (i) Q159H, (ii) Y286I, and (iii) Q293M or Q293V; (i) Q159H, (ii) Y286V, and (iii) Q293F, Q293M, Q293V, or Q293W; (i) Q159L, (ii) S175H, and (iii) Q293F; (i) S175H, (ii), Y286V, and (iii) Q293M; (i) S175H, (ii), Y286I, and (iii) Q293M or Q293V; (i) Q159S, (ii) S175H, and (iii) Y286I; (i) Q159S, (ii) S175R, and (iii) Y286V; (i) Q159S, (ii) S175S, and (iii) Y286I; and (i) Q159S, (ii) S212H, and (iii) Y286A or Y286V.

In some aspects, the non-natural prenyltransferase is based on template SEQ ID NO: 16 or has 50% or greater identity, 60% or greater identity, 65% or greater identity, 70% or greater identity, 75% or greater identity, 80% or greater identity, 85% or greater identity, 87.5% or greater identity, 90% or greater identity, 92.5% or greater identity, or 95% or greater identity, to SEQ ID NO: 16 and has four or more amino acid variations at position(s) selected from, but not limited to, the group consisting of (i) Q159H, (ii) S175H, (iii) Y286A, and (iv) Q293V; (i) Q159H, (ii) S175H, (iii) Y286V, and (iv) Q293M or Q293V; (i) Q159H, (ii) S175R, (iii) Y286I, and (iv) Q293M; (i) Q159L, (ii) S175K, (iii) Y286A, and (iv) Q293V; (i) Q159M, (ii) S175H, (iii) Y286V, and (iv) Q293F; (i) Q159R, (ii) S175H, (iii) Y286I, and (iv) Q293Q; (i) Q159S, (ii) S175H, (iii) Y286V, and (iv) Q293F; (i) Q159S, (ii) S175K, (iii) Y286V, and (iv) Q293V; and (i) Q159S, (ii) S212H, (iii) Y286V, and (iv) Q293M.

In other aspects, the prenyltransferase template is any of SEQ ID NOs: 1-15, 17-27, or a homolog thereof, that include these variations at the corresponding positions. For example, in SEQ ID NO: 1 (NphB), the variant positions are shifted +2, compared to the orthologous positions shown above for SEQ ID NO:16. Accordingly, expressly contemplated for each template, the non-natural prenyltransferase is based on any one of the template SEQ ID NOs: 1-15, 17-27 or has 50% or greater identity, 60% or greater identity, 65% or greater identity, 70% or greater identity, 75% or greater identity, 80% or greater identity, 85% or greater identity, 87.5% or greater identity, 90% or greater identity, 92.5% or greater identity, or 95% or greater identity, to the template selected from SEQ ID NOs: 1-15, 17-27, and has four or more amino acid variations at position(s) selected from, but not limited to, the group consisting of the following based on SEQ ID NO: 16: (i) Q159H, (ii) S175H, (iii) Y286A, and (iv) Q293V; (i) Q159H, (ii) S175H, (iii) Y286V, and (iv) Q293M or Q293V; (i) Q159H, (ii) S175R, (iii) Y286I, and (iv) Q293M; (i) Q159L, (ii) S175K, (iii) Y286A, and (iv) Q293V; (i) Q159M, (ii) S175H, (iii) Y286V, and (iv) Q293F; (i) Q159R, (ii) S175H, (iii) Y286I, and (iv) Q293Q; (i) Q159S, (ii) S175H, (iii) Y286V, and (iv) Q293F; (i) Q159S, (ii) S175K, (iii) Y286V, and (iv) Q293V; and (i) Q159S, (ii) S212H, (iii) Y286V, and (iv) Q293M.

In other aspects, the non-natural prenyltransferase is based on template SEQ ID NO: 16 or has 50% or greater identity, 60% or greater identity, 65% or greater identity, 70% or greater identity, 75% or greater identity, 80% or greater identity, 85% or greater identity, 87.5% or greater identity, 90% or greater identity, 92.5% or greater identity, or 95% or greater identity, to SEQ ID NO: 16 and has five or more amino acid variations at position(s) selected from, but not limited to, the group consisting of (i) Q159H, (ii) S175R, (iii) S212H, (iv) Y286A, and (v) Q293V; and (i) Q159R, (ii) S175R, (iii) S212H, (iv) Y286I, and (v) Q293M.

In other aspects, the prenyltransferase template is any of SEQ ID NO: 1-15, 17-27, or a homolog thereof, that include these variations at the corresponding positions. For example, in SEQ ID NO: 1 (NphB), the variant positions are shifted +2, compared to the orthologous positions shown above for SEQ ID NO:16. Accordingly, expressly contemplated for each template, the non-natural prenyltransferase is based on any one of the template SEQ ID NOs: 1-15, 17-27 or has 50% or greater identity, 60% or greater identity, 65% or greater identity, 70% or greater identity, 75% or greater identity, 80% or greater identity, 85% or greater identity, 87.5% or greater identity, 90% or greater identity, 92.5% or greater identity, or 95% or greater identity, to the template selected from SEQ ID NOs: 1-15, 17-27, and has five or more amino acid variations at position(s) selected from the group consisting of positions selected from the group consisting of the following based on SEQ ID NO: 16: (i) Q159H, (ii) S175R, (iii) S212H, (iv) Y286A, and (v) Q293V; and (i) Q159R, (ii) S175R, (iii) S212H, (iv) Y286I, and (v) Q293M.

Optionally, the non-natural prenyltransferase of the disclosure can further include, in addition to the one or more variant amino acids as described herein, one or more amino acid variations at positions selected from: F211N, F211 S, A230S, G284S, and Y286N, relative to SEQ ID NO: 16; or F213N, F213S, A232S, G286S, and Y288N, relative to SEQ ID NO: 1 (NphB). See, for example, Valliere, M. A., et al. (2019) Nature Communications, 10; 565.

Site-directed mutagenesis or sequence alteration (e.g., site-specific mutagenesis or oligonucleotide-directed) can be used to make specific changes to a target prenyltransferase DNA sequence to provide a variant DNA sequence encoding prenyltransferase with the desired amino acid substitution. As a general matter, an oligonucleotide having a sequence that provides a codon encoding the variant amino acid may be used. Alternatively, artificial gene sequence of the entire coding region of the variant prenyltransferase DNA sequence can be performed as preferred prenyltransferase targeted for substitution are generally less than 400 amino acids long.

Exemplary techniques using mutagenic oligonucleotides for generation of a variant prenyltransferase sequence include the Kunkel method which may utilize a prenyltransferase gene sequence placed into a phagemid. The phagemid in E. coli produces prenyltransferase ssDNA which is the template for mutagenesis using an oligonucleotide which is a primer extended on the template.

Depending on the restriction enzyme sites flanking a location of interest in the prenyltransferase DNA, cassette mutagenesis may be used to create a variant sequence of interest. For cassette mutagenesis, a DNA fragment is synthesized, inserted into a plasmid, cleaved with a restriction enzyme, and then subsequently ligated to a pair of complementary oligonucleotides containing the prenyltransferase variant mutation. The restriction fragments of the plasmid and oligonucleotide can be ligated to one another.

Another technique that can be used to generate the variant prenyltransferase sequence is PCR site directed mutagenesis. Mutagenic oligonucleotide primers are used to introduce the desired mutation and to provide a PCR fragment carrying the mutated sequence.

Additional oligonucleotides may be used to extend the ends of the mutated fragment to provide restriction sites suitable for restriction enzyme digestion and insertion into the gene.

Commercial kits for site-directed mutagenesis techniques are also available. For example, the Quikchange™ kit uses complementary mutagenic primers to PCR amplify a gene region using a high-fidelity non-strand-displacing DNA polymerase such as pfu polymerase. The reaction generates a nicked, circular DNA which is relaxed. The template DNA is eliminated by enzymatic digestion with a restriction enzyme such as Dpnl which is specific for methylated DNA.

An expression vector or vectors can be constructed to include one or more variant prenyltransferase encoding nucleic acids as exemplified herein operably linked to expression control sequences functional in the host organism. Expression vectors applicable for use in the microbial host organisms provided include, for example, plasmids, phage vectors, viral vectors, episomes and artificial chromosomes, including vectors and selection sequences or markers operable for stable integration into a host chromosome. Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more exogenous encoding nucleic acids are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The transformation of exogenous nucleic acid sequences involved in a metabolic or synthetic pathway can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the exogenous nucleic acid is expressed in a sufficient amount to produce the desired product, and it is further understood that expression levels can be optimized to obtain sufficient expression using methods well known in the art and as disclosed herein.

The term “exogenous” is intended to mean that the referenced molecule or the referenced activity is introduced into the host microbial organism. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the microbial organism. When used in reference to a biosynthetic activity, the term refers to an activity that is introduced into the host reference organism. The source can be, for example, a homologous or heterologous encoding nucleic acid that expresses the referenced activity following introduction into the host microbial organism. Therefore, the term “endogenous” refers to a referenced molecule or activity that is present in the host. Similarly, the term when used in reference to expression of an encoding nucleic acid refers to expression of an encoding nucleic acid contained within the microbial organism. The term “heterologous” refers to a molecule or activity derived from a source other than the referenced species whereas “homologous” refers to a molecule or activity derived from the host microbial organism. Accordingly, exogenous expression of an encoding nucleic acid can utilize either or both a heterologous or homologous encoding nucleic acid.

It is understood that when more than one exogenous nucleic acid is included in a microbial organism, the more than one exogenous nucleic acid(s) refers to the referenced encoding nucleic acid or biosynthetic activity, as discussed above. It is further understood, as disclosed herein, that more than one exogenous nucleic acid(s) can be introduced into the host microbial organism on separate nucleic acid molecules, on polycistronic nucleic acid molecules, or a combination thereof, and still be considered as more than one exogenous nucleic acid. For example, as disclosed herein a microbial organism can be engineered to express two or more exogenous nucleic acids encoding a desired pathway enzyme or protein. In the case where two exogenous nucleic acids encoding a desired activity are introduced into a host microbial organism, it is understood that the two exogenous nucleic acids can be introduced as a single nucleic acid, for example, on a single plasmid, on separate plasmids, can be integrated into the host chromosome at a single site or multiple sites, and still be considered as two exogenous nucleic acids. Similarly, it is understood that more than two exogenous nucleic acids can be introduced into a host organism in any desired combination, for example, on a single plasmid, on separate plasmids, can be integrated into the host chromosome at a single site or multiple sites, and still be considered as two or more exogenous nucleic acids, for example three exogenous nucleic acids. Thus, the number of referenced exogenous nucleic acids or biosynthetic activities refers to the number of encoding nucleic acids or the number of biosynthetic activities, not the number of separate nucleic acids introduced into the host organism.

Exogenous variant prenyltransferase-encoding nucleic acid sequences can be introduced stably or transiently into a host cell using techniques well known in the art including, but not limited to, conjugation, electroporation, chemical transformation, transduction, transfection, and ultrasound transformation. Optionally, for exogenous expression in E. coli or other prokaryotic cells, some nucleic acid sequences in the genes or cDNAs of eukaryotic nucleic acids can encode targeting signals such as an N-terminal mitochondrial or other targeting signal, which can be removed before transformation into prokaryotic host cells, if desired. For example, removal of a mitochondrial leader sequence leads to increased expression in E. coli (Hofmeister et ah, J. Biol. Chem. 280:4329-4338 (2005)). For exogenous expression in yeast or other eukaryotic cells, genes can be expressed in the cytosol without the addition of leader sequence, or can be targeted to mitochondrion or other organelles, or targeted for secretion, by the addition of a suitable targeting sequence such as a mitochondrial targeting or secretion signal suitable for the host cells. Thus, it is understood that appropriate modifications to a nucleic acid sequence to remove or include a targeting sequence can be incorporated into an exogenous nucleic acid sequence to impart desirable properties. Furthermore, genes can be subjected to codon optimization with techniques well known in the art to achieve optimized expression of the proteins.

The terms “microbial,” “microbial organism” or “microorganism” are intended to mean any organism that exists as a microscopic cell that is included within the domains of archaea, bacteria or eukarya. Therefore, the term is intended to encompass prokaryotic or eukaryotic cells or organisms having a microscopic size and includes bacteria, archaea and eubacteria of all species as well as eukaryotic microorganisms such as yeast and fungi. The term also includes cell cultures of any species that can be cultured for the production of a biochemical.

The term “isolated” when used in reference to a microbial organism is intended to mean an organism that is substantially free of at least one component that the referenced microbial organism is found with in nature. The term includes a microbial organism that is removed from some or all components as it is found in its natural environment. The term also includes a microbial organism that is removed from some or all components as the microbial organism is found in non-naturally occurring environments.

In some aspects, the prenyltransferase variant gene is introduced into a cell with a gene disruption. The term “gene disruption,” or grammatical equivalents thereof, is intended to mean a genetic alteration that renders the encoded gene product inactive or attenuated. The genetic alteration can be, for example, deletion of the entire gene, deletion of a regulatory sequence required for transcription or translation, deletion of a portion of the gene which results in a truncated gene product, or by any of various mutation strategies that inactivate or attenuate the encoded gene product. One particularly useful method of gene disruption is complete gene deletion because it reduces or eliminates the occurrence of genetic reversions. The phenotypic effect of a gene disruption can be a null mutation, which can arise from many types of mutations including inactivating point mutations, entire gene deletions, and deletions of chromosomal segments or entire chromosomes. Specific antisense nucleic acid compounds and enzyme inhibitors, such as antibiotics, can also produce null mutant phenotype, therefore being equivalent to gene disruption.

A metabolic modification refers to a biochemical reaction that is altered from its naturally occurring state. Therefore, microorganisms may have genetic modifications to nucleic acids encoding metabolic polypeptides, or functional fragments thereof. Exemplary metabolic modifications are disclosed herein.

The microorganisms provided herein can contain stable genetic alterations, which refers to microorganisms that can be cultured for greater than five generations without loss of the alteration. Generally, stable genetic alterations include modifications that persist greater than 10 generations, particularly stable modifications will persist more than about 25 generations, and more particularly, stable genetic modifications will be greater than 50 generations, including indefinitely.

Those skilled in the art will understand that the genetic alterations, including metabolic modifications exemplified herein, are described with reference to a suitable host organism such as E. coli and their corresponding metabolic reactions or a suitable source organism for desired genetic material such as genes for a desired metabolic pathway.

However, given the complete genome sequencing of a wide variety of organisms and the high level of skill in the area of genomics, those skilled in the art will readily be able to apply the teachings and guidance provided herein to essentially all other organisms. For example, the E. coli metabolic alterations exemplified herein can readily be applied to other species by incorporating the same or analogous encoding nucleic acid from species other than the referenced species. Such genetic alterations include, for example, genetic alterations of species homologs, in general, and in particular, orthologs, paralogs or non-orthologous gene displacements.

A variety of microorganisms may be suitable for incorporating the variant prenyltransferase, optionally with one or more other transgenes. Such organisms include both prokaryotic and eukaryotic organisms including, but not limited to, bacteria, including archaea and eubacteria, and eukaryotes, including yeast, plant, insect, animal, and mammal, including human. Exemplary species are reported in U.S. application Ser. No. 13/975,678 (filed Aug. 26, 2013), which is incorporated herein by reference, and include, for example, Escherichia coli, Saccharomyces cerevisiae, Saccharomyces kluyveri, Candida boidinii, Clostridium kluyveri, Clostridium acetobutylicum, Clostridium beijerinckii, Clostridium saccharoperbutylacetonicum, Clostridium perfringens, Clostridium difficile, Clostridium botulinum, Clostridium tyrobutyricum, Clostridium tetanomorphum, Clostridium tetani, Clostridium propionicum, Clostridium aminobutyricum, Clostridium subterminale, Clostridium sticklandii, Ralstonia eutropha, Mycobacterium bovis, Mycobacterium tuberculosis, Porphyromonas gingivalis, Arabidopsis thaliana, Thermus thermophilus, Pseudomonas species, including Pseudomonas aeruginosa, Pseudomonas putida, Pseudomonas stutzeri, Pseudomonas fluorescens, Homo sapiens, Oryctolagus cuniculus, Rhodobacter spaeroides, Thermoanaerobacter brockii, Metallosphaera sedula, Leuconostoc mesenteroides, Chloroflexus aurantiacus, Roseiflexus castenholzii, Erythrobacter, Simmondsia chinensis, Acinetobacter species, including Acinetobacter calcoaceticus and Acinetobacter baylyi, Porphyromonas gingivalis, Sulfolobus tokodaii, Sulfolobus solfataricus, Sulfolobus acidocaldarius, Bacillus subtilis, Bacillus cereus, Bacillus megaterium, Bacillus brevis, Bacillus pumilus, Rattus norvegicus, Klebsiella pneumonia, Klebsiella oxytoca, Euglena gracilis, Treponema denticola, Moorella thermoacetica, Thermotoga maritima, Halobacterium salinarum, Geobacillus stearothermophilus, Aeropyrum pernix, Sus scrofa, Caenorhabditis elegans, Corynebacterium glutamicum, Acidaminococcus fermentans, Lactococcus lactis, Lactobacillus plantarum, Streptococcus thermophilus, Enterobacter aerogenes, Candida, Aspergillus terreus, Pedicoccus pentosaceus, Zymomonas mobilus, Acetobacter pasteurians, Kluyveromyces lactis, Eubacterium barkeri, Bacteroides capillosus, Anaerotruncus colihominis, Natranaerobius thermophilusm, Campylobacter jejuni, Haemophilus influenzae, Serratia marcescens, Citrobacter amalonaticus, Myxococcus xanthus, Fusobacterium nuleatum, Penicillium chrysogenum, marine gamma proteobacterium, butyrate producing bacterium, Nocardia iowensis, Nocardia farcinica, Streptomyces griseus, Schizosaccharomyces pombe, Geobacillus thermoglucosidasius, Salmonella typhimurium, Vibrio cholera, Heliobacter pylori, Nicotiana tabacum, Oryza sativa, Haloferax mediterranei, Agrobacterium tumefaciens, Achromobacter denitrifwans, Fusobacterium nucleatum, Streptomyces clavuligenus, Acinetobacter baumanii, Mus musculus, Lachancea kluyveri, Trichomonas vaginalis, Trypanosoma brucei, Pseudomonas stutzeri, Bradyrhizobium japonicum, Mesorhizobium loti, Bos taurus, Nicotiana glutinosa, Vibrio vulnificus, Selenomonas ruminantium, Vibrio parahaemolyticus, Archaeoglobus fulgidus, Haloarcula marismortui, Pyrobaculum aerophilum, Mycobacterium smegmatis MC2 155, Mycobacterium avium subsp. paratuberculosis K-10, Mycobacterium marinum M, Tsukamurella paurometabola DSM 20162, Cyanobium PCC7001, Dictyostelium discoideum AX4, as well as other exemplary species disclosed herein or available as source organisms for corresponding genes.

In certain aspects, suitable organisms include Acinetobacter baumannii Naval-82, Acinetobacter sp. ADP1, Acinetobacter sp. strain M-1, Actinobacillus succinogenes 130Z, Allochromatium vinosum DSM 180, Amycolatopsis methanolica, Arabidopsis thaliana, Atopobium parvulum DSM 20469, Azotobacter vinelandii DJ Bacillus alcalophilus ATCC 27647, Bacillus azotoformans LMG 9581, Bacillus coagulans 36D1, Bacillus megaterium, Bacillus methanolicus MGA3, Bacillus methanolicus PB1, Bacillus methanolicus PB-1, Bacillus selenitireducens MLS 10, Bacillus smithii, Bacillus subtilis, Burkholderia cenocepacia, Burkholderia cepacia, Burkholderia multivorans, Burkholderia pyrrocinia, Burkholderia stabilis, Burkholderia thailandensis E264, Burkholderiales bacterium Joshi 001, Butyrate—producing bacterium L2-50, Campylobacter jejuni, Candida albicans, Candida boidinii, Candida methylica, Carboxydothermus hydrogenoformans, Carboxydothermus hydrogenoformans Z-2901, Caulobacter sp. AP07, Chloroflexus aggregans DSM 9485, Chloroflexus aurantiacus J-10-fl, Citrobacter freundii, Citrobacter koseri ATCC BAA-895, Citrobacter youngae, Clostridium, Clostridium acetobutylicum, Clostridium acetobutylicum ATCC 824, Clostridium acidurici, Clostridium aminobutyricum, Clostridium asparagiforme DSM 15981, Clostridium beijerinckii, Clostridium beijerinckii NCIMB 8052, Clostridium bolteae ATCC BAA-613, Clostridium carboxidivorans P7, Clostridium cellulovorans 743B, Clostridium difficile, Clostridium hiranonis DSM 13275, Clostridium hylemonae DSM 15053, Clostridium kluyveri, Clostridium kluyveri DSM 555, Clostridium ljungdahli, Clostridium ljungdahlii DSM 13528, Clostridium methylpentosum DSM 5476, Clostridium pasteur ianum, Clostridium pasteurianum DSM 525, Clostridium perfringens, Clostridium perfringens ATCC 13124, Clostridium perfringens str. 13, Clostridium phytofermentans ISDg, Clostridium saccharobutylicum, Clostridium saccharoperbutylacetonicum, Clostridium saccharoperbutylacetonicum N1-4, Clostridium tetani, Corynebacterium glutamicum ATCC 14067, Corynebacterium glutamicum R, Corynebacterium sp. U-96, Corynebacterium variabile, Cupriavidus necator N-1, Cyanobium PCC7001, Desulfatibacillum alkenivorans AK-01, Desulfitobacterium hafniense, Desulfitobacterium metallireducens DSM 15288, Desulfotomaculum reducens MI-1, Desulfovibrio africanus str. Walvis Bay, Desulfovibrio fructosovorans JJ, Desulfovibrio vulgaris str. Hildenborough, Desulfovibrio vulgaris str. Miyazaki F′, Dictyostelium discoideum AX4, Escherichia coli, Escherichia coli K-12, Escherichia coli K-12 MG 1655, Eubacterium hallii DSM 3353, Flavobacterium frigoris, Fusobacterium nucleatum subsp. polymorphum ATCC 10953, Geobacillus sp. Y4.1MC1, Geobacillus themodenitrifwans NG80-2, Geobacter bemidjiensis Bern, Geobacter sulfurreducens, Geobacter sulfur reducens PCA, Geobacillus stearothermophilus DSM 2334, Haemophilus influenzae, Helicobacter pylori, Homo sapiens, Hydrogenobacter thermophilus, Hydrogenobacter thermophilus TK-6, Hyphomicrobium denitrificans ATCC 51888, Hyphomicrobium zavarzinii, Klebsiella pneumoniae, Klebsiella pneumoniae subsp. pneumoniae MGH 78578, Lactobacillus brevis ATCC 367, Leuconostoc mesenteroides, Lysinibacillus fusiformis, Lysinibacillus sphaericus, Mesorhizobium loti MAFF 303099, Metallosphaera sedula, Methanosarcina acetivorans, Methanosarcina acetivorans C2A, Methanosarcina barkeri, Methanosarcina mazei TucOl, Methylobacter marinus, Methylobacterium extorquens, Methylobacterium extorquens AMI, Methylococcus capsulatas, Methylomonas aminofaciens, Moorella thermoacetica, Mycobacter sp. strain JC1 DSM 3803, Mycobacterium avium subsp. paratuberculosis K-10, Mycobacterium bovis BCG, Mycobacterium gastri, Mycobacterium marinum M, Mycobacterium smegmatis, Mycobacterium smegmatis MC2 155, Mycobacterium tuberculosis, Nitrosopumilus salaria BD31, Nitrososphaera gargensis Ga9.2, Nocar dia farcinica IFM 10152, Nocardia iowensis (sp. NRRL 5646), Nostoc sp. PCC 7120, Ogataea angusta, Ogataea par apolymorpha DL-1 (Hansenula polymorpha DL-1), Paenibacillus peoriae KCTC 3763, Paracoccus denitrificans, Penicillium chrysogenum, Photobacterium profundum 3TCK, Phytofermentans ISDg, Pichia pastoris, Picrophilus torridus DSM9790, Porphyromonas gingivalis, Porphyromonas gingivalis W83, Pseudomonas aeruginosa P AO 1, Pseudomonas denitrificans, Pseudomonas knackmussii, Pseudomonas putida, Pseudomonas sp, Pseudomonas syringae pv. syringae B728a, Pyrobaculum islandicum DSM 4184, Pyrococcus abyssi, Pyrococcus furiosus, Pyrococcus horikoshii OT3, Ralstonia eutropha, Ralstonia eutropha HI 6, Rhodobacter capsulatus, Rhodobacter sphaeroides, Rhodobacter sphaeroides ATCC 17025, Rhodopseudomonas palustris, Rhodopseudomonas palustris CGA009, Rhodopseudomonas palustris DX-1, Rhodospirillum rubrum, Rhodospirillum rubrum ATCC 11170, Ruminococcus obeum ATCC 29174, Saccharomyces cerevisiae, Saccharomyces cerevisiae S288c, Salmonella enterica, Salmonella enterica subsp. enterica serovar Typhimurium str. LT2, Salmonella enterica typhimurium, Salmonella typhimurium, Schizosaccharomyces pombe, Sebaldella termitidis ATCC 33386, Shewanella oneidensis MR-1, Sinorhizobium meliloti 1021, Streptomyces coelicolor, Streptomyces griseus subsp. griseus NBRC 13350, Sulfolobus acidocalarius, Sulfolobus solfataricus P-2, Synechocystis str. PCC 6803, Syntrophobacter fumaroxidans, Thauera aromatica, Thermoanaerobacter sp. X514, Thermococcus kodakaraensis, Thermococcus litoralis, Thermoplasma acidophilum, Thermoproteus neutrophilus, Thermotoga maritima, Thiocapsa roseopersicina, Tolumonas auensis DSM 9187, Trichomonas vaginalis G3, Trypanosoma brucei, Tsukamurella paurometabola DSM 20162, Vibrio cholera, Vibrio harveyi ATCC BAA-1116, Xanthobacter autotrophicus Py2, Yersinia intermedia, or Zea mays.

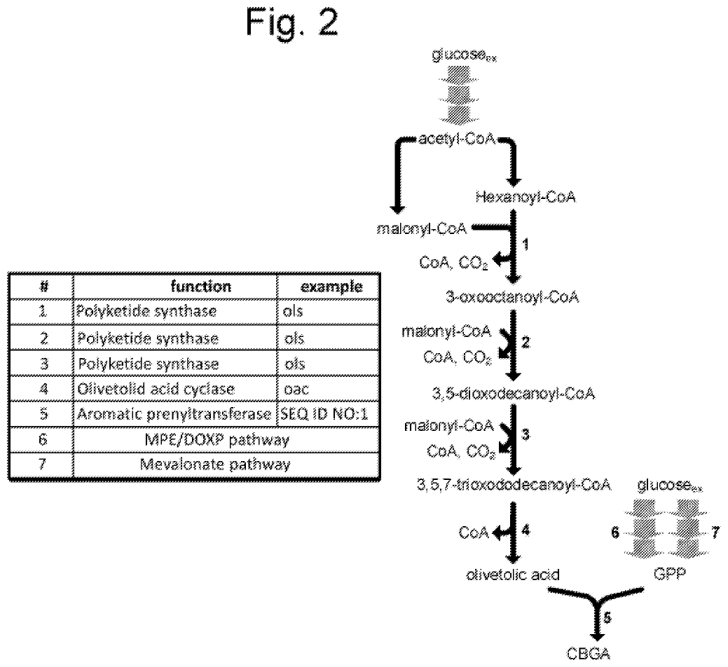

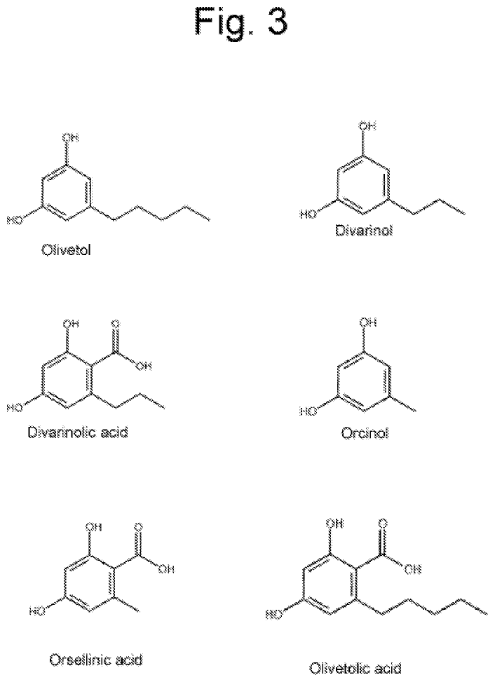

FIG. 2 shows exemplary pathways to CBGA formation from hexanoyl-CoA, and geranyl diphosphate. In some cases, the engineered cell of the disclosure can utilize hexanoyl-CoA that is produced from a cellular fatty acid biosynthesis pathway. For example, hexanoyl-CoA can be formed endogenously via reverse beta-oxidation of fatty acids.

In other aspects, the engineered cell can further include hexanoyl-CoA synthetase, such as expressed on a transgene. Exemplary hexanoyl-CoA synthetase genes include enzymes endogenous to bacteria, including E. coli, as well as eukaryotes, including yeast and C. sativa (see for example Stout et al, Plant J., 2012; 71:353-365).

FIG. 2 also shows pathway formation of malonyl-CoA, which is used for the formation of olivetolic acid along with hexanoyl-CoA. Endogenous malonyl-CoA formation can be supplemented by formation from acetyl CoA using overexpression of acetyl-CoA carboxylase. Accordingly, the engineered cell can further include acetyl-CoA carboxylase, such as expressed on a transgene or integrated into the genome.

Acetyl-CoA carboxylase (EC 6.4.1.2) catalyzes the ATP-dependent carboxylation of acetyl-CoA to malonyl-CoA. This enzyme is biotin dependent and is the first reaction of fatty acid biosynthesis initiation in several organisms.

TABLE 2
Exemplary enzymes are encoded by accABCD of E. coli
(Davis et al, J Biol Chem 275: 28593-8 (2000)), ACC1
of Saccharomyces cerevisiae and homologs (Sumper
et al, Methods Enzym 71: 34-7 (1981)).

| Protein | GenBank ID | GI Number | Organism |
|---|---|---|---|
| ACC1 | CAA96294.1 | 1302498 | Saccharomyces |
| | | | cerevisiae |
| KLLA0F06072g | XP_455355.1 | 50310667 | Kluyveromyces |
| | | | lactis |
| ACC1 | XP_718624.1 | 68474502 | Candida |
| | | | albicans |
| YALI0C11407p | XP_501721.1 | 50548503 | Yarrowia |
| | | | lipolytica |
| ANI_1_1724104 | XP_001395476.1 | 145246454 | Aspergillus |
| | | | niger |
| accA | AAC73296.1 | 1786382 | Escherichia |
| | | | coli |
| accB | AAC76287.1 | 1789653 | Escherichia |
| | | | coli |
| accC | AAC76288.1 | 1789654 | Escherichia |
| | | | coli |
| accD | AAC75376.1 | 1788655 | Escherichia |
| | | | coli |

FIG. 2 also shows polyketide synthase converts hexanoyl-CoA to olivetolic acid through poly-keto intermediates. Accordingly, the engineered cell can further include polyketide synthase, such as expressed on a transgene or integrated into the genome. The engineered cell can further include olivetolic acid cyclase (oac), to convert 3,5,7-trioxododecanoyl-CoA to olivetolic acid.

In some aspects, the engineered cell preferentially uses a 5-alkylbenzene-1,3-diol as an (alcohol) substrate instead of an acid derivative of an alkylbenzene-1,3-diol. The 5-alkylbenzene-1,3-diol can be reacted with GPP to form a 2-prenylated 5-alkylbenzene-1,3-diol. For example, reaction of olivetol and GPP promoted with the non-natural prenyltransferase variants of the disclosure can form cannabigerol (CBG; 2-GOL).

Accordingly, formation of the acid derivative of an alkylbenzene-1,3-diol can be avoided in cell. To avoid formation of the acid derivative, the olivetolic acid cyclase (oac) gene can be excluded from the pathway, or can be deleted from the cell. Gagne, S. J. et al (PNAS, 109: 12811-12816, 2012) describes a pathway utilizing hexanoyl-CoA which can be converted to olivetol using tetraketide synthase (TKS), or further to olivetolic acid by action of olivetolic acid cyclase (oac).

Optionally, the engineered cell can include one or more exogenous genes which allow the cell to grow on carbon sources the cell would not normally metabolize, or one or more exogenous genes or modifications to endogenous genes that allow the cell to have improved growth on carbon sources the cell normally uses. For example, WO2015/051298 (MDH variants) and WO2017/075208 (MDH fusions) describe genetic modifications that provide pathways allowing to cell to grow on methanol; WO2009/094485 (syngas) describes genetic modifications that provide pathways allowing to cell to grow on synthesis gas.

As used herein, the term “bioderived” means derived from or synthesized by a biological organism and can be considered a renewable resource since it can be generated by a biological organism. Such a biological organism, in particular the microbial organisms disclosed herein, can utilize feedstock or biomass, such as, sugars or carbohydrates obtained from an agricultural, plant, bacterial, or animal source. Alternatively, the biological organism can utilize atmospheric carbon. As used herein, the term “biobased” means a product as described above that is composed, in whole or in part, of a bioderived compound of the disclosure. A biobased or bioderived product is in contrast to a petroleum derived product, wherein such a product is derived from or synthesized from petroleum or a petrochemical feedstock.

Depending on the desired microorganism or strain to be used, the appropriate culture medium may be used. For example, descriptions of various culture media may be found in “Manual of Methods for General Bacteriology” of the American Society for Bacteriology (Washington D.C., USA, 1981). As used herein, “medium” as it relates to the growth source refers to the starting medium be it in a solid or liquid form. “Cultured medium”, on the other hand and as used herein refers to medium (e.g., liquid medium) containing microbes that have been fermentatively grown and can include other cellular biomass. The medium generally includes one or more carbon sources, nitrogen sources, inorganic salts, vitamins and/or trace elements.

Exemplary carbon sources include sugar carbons such as sucrose, glucose, galactose, fructose, mannose, isomaltose, xylose, pannose, maltose, arabinose, cellobiose and 3-, 4-, or 5-oligomers thereof. Other carbon sources include alcohol carbon sources such as methanol, ethanol, glycerol, formate and fatty acids. Still other carbon sources include carbon sources from gas such as synthesis gas, waste gas, methane, CO, CO₂ and any mixture of CO, CO₂ with H₂. Other carbon sources can include renewal feedstocks and biomass. Exemplary renewal feedstocks include cellulosic biomass, hemicellulosic biomass and lignin feedstocks.

In some aspects, culture conditions include anaerobic or substantially anaerobic growth or maintenance conditions. Exemplary anaerobic conditions have been described previously and are well known in the art. Exemplary anaerobic conditions for fermentation processes are disclosed, for example, in U.S. Patent Application Publication No 2009/0047719, filed Aug. 10, 2007. Any of these conditions can be employed with the microbial organisms as well as other anaerobic conditions well known in the art.

The culture conditions can include, for example, liquid culture procedures as well as fermentation and other large-scale culture procedures. Useful yields of the products can be obtained under anaerobic or substantially anaerobic culture conditions.

An exemplary growth condition for achieving one or more cannabinoid product(s) includes anaerobic culture or fermentation conditions. In certain aspects, the microbial organism can be sustained, cultured or fermented under anaerobic or substantially anaerobic conditions. Briefly, anaerobic conditions refer to an environment devoid of oxygen.

Substantially anaerobic conditions include, for example, a culture, batch fermentation or continuous fermentation such that the dissolved oxygen concentration in the medium remains between 0 and 10% of saturation. Substantially anaerobic conditions also includes growing or resting cells in liquid medium or on solid agar inside a sealed chamber maintained with an atmosphere of less than 1% oxygen. The percent of oxygen can be maintained by, for example, sparging the culture with an N₂/CO₂ mixture or other suitable non-oxygen gas or gases.

The culture conditions can be scaled up and grown continuously for manufacturing cannabinoid product. Exemplary growth procedures include, for example, fed-batch fermentation and batch separation; fed-batch fermentation and continuous separation, or continuous fermentation and continuous separation. All of these processes are well known in the art. Fermentation procedures are particularly useful for the biosynthetic production of commercial quantities of cannabinoid product. Generally, and as with non-continuous culture procedures, the continuous and/or near-continuous production of cannabinoid product will include culturing a cannabinoid producing organism on sufficient nutrients and medium to sustain and/or nearly sustain growth in an exponential phase. Continuous culture under such conditions can include, for example, 1 day, 2, 3, 4, 5, 6 or 7 days or more. Additionally, continuous culture can include 1 week, 2, 3, 4 or 5 or more weeks and up to several months. Alternatively, the desired microorganism can be cultured for hours, if suitable for a particular application. It is to be understood that the continuous and/or near-continuous culture conditions also can include all time intervals in between these exemplary periods. It is further understood that the time of culturing the microbial organism is for a sufficient period of time to produce a sufficient amount of product for a desired purpose.

Fermentation procedures are well known in the art. Briefly, fermentation for the biosynthetic production of cannabinoid product can be utilized in, for example, fed-batch fermentation and batch separation; fed-batch fermentation and continuous separation, or continuous fermentation and continuous separation. Examples of batch and continuous fermentation procedures are well known in the art.

The culture medium at the start of fermentation may have a pH of about 5 to about 7. The pH may be less than 11, less than 10, less than 9, or less than 8. In other aspects, the pH may be at least 2, at least 3, at least 4, at least 5, at least 6, or at least 7. In other aspects, the pH of the medium may be about 6 to about 9.5; 6 to about 9, about 6 to 8 or about 8 to 9.

Suitable purification and/or assays to test, e.g., for the production of 3-geranyl-olivetolate can be performed using well known methods. Suitable replicates such as triplicate cultures can be grown for each engineered strain to be tested. For example, product and byproduct formation in the engineered production host can be monitored. The final product and intermediates, and other organic compounds, can be analyzed by methods such as HPLC (High Performance Liquid Chromatography), GC-MS (Gas Chromatography-Mass Spectroscopy) and LC-MS (Liquid Chromatography-Mass Spectroscopy) or other suitable analytical methods using routine procedures well known in the art. The release of product in the fermentation broth can also be tested with the culture supernatant. Byproducts and residual glucose can be quantified by HPLC using, for example, a refractive index detector for glucose and alcohols, and a UV detector for organic acids (Lin et ah, Biotechnol. Bioeng. 90:775-779 (2005)), or other suitable assay and detection methods well known in the art. The individual enzyme or protein activities from the exogenous DNA sequences can also be assayed using methods well known in the art.

The 3-geranyl-olivetolate (CBGA) or other target molecules may be separated from other components in the culture using a variety of methods well known in the art. Such separation methods include, for example, extraction procedures as well as methods that include continuous liquid-liquid extraction, pervaporation, evaporation, filtration, membrane filtration (including reverse osmosis, nanofiltration, ultrafiltration, and microfiltration), membrane filtration with diafiltration, membrane separation, reverse osmosis, electrodialysis, distillation, extractive distillation, reactive distillation, azeotropic distillation, crystallization and recrystallization, centrifugation, extractive filtration, ion exchange chromatography, size exclusion chromatography, adsorption chromatography, carbon adsorption, hydrogenation, and ultrafiltration. All of the above methods are well known in the art.

In view of the regioselectivity of the prenyltransferase variants, the disclosure also provides compositions that are enriched for desired cannabinoids and derivatives thereof. In particular, the disclosure provides compositions enriched for CBGA (3-geranyl-olivetolate (3-GOLA)) and/or CBG compared to the undesired isomer, e.g., 5-GOLA or 4-GOL (decarboxylated 5-GOLA). Such enriched compositions include those that are pharmaceutical compositions as well as those that are used for non-pharmaceutical purposes, including medicinal purposes. Accordingly, in some aspects, provided are compositions, such as pharmaceutical compositions or medicinal compositions, with CBGA and/or CBG that are 90% or greater, 91% or greater, 92% or greater, 93% or greater, 94% or greater, 95% or greater, 96% or greater, 97% or greater, 98% or greater, 99% or greater, 99.2% or greater, 99.4% or greater, 99.5% or greater, 99.6% or greater, 99.7% or greater, 99.8% or greater, 99.9% or greater, 99.95% or greater or even 100% 3-geranyl-olivetolate (3-GOLA) or its decarboxylated derivative CBG (2-GOL), of all geranyl-olivetolate compounds, including 5-GOLA and 4-GOL compounds, which can be less desirable when present in various compositions.

### EXAMPLES

### Example 1. Library Constructs and Strains

Mutant variants of prenyltransferase were constructed as libraries on plasmid by single-site and multi-site (combinatorial) mutagenesis methods, using specific primers at the positions undergoing mutagenesis, amplifying fragments via PCR, and circularizing plasmid via Gibson ligation. Prenyltransferase variants identified by BLAST searches were codon optimized for expression in E. coli. Gene blocks were synthesized from Integrated DNA Technologies (San Diego, Calif.) and cloned into pD441-SR by Gibson Assembly (NEB Catalog #E2611L). Some DNA plasmids encoding variants of homologs were ordered and delivered in the background of the T5 expression vector pD441-SR from DNA2.0 (now ATUM, catalog pD441-SR). All other mutational variants and wild-type homologs were created using site directed mutagenesis with QuikChange II Site-Directed Mutagenesis Kit (Agilent catalog #200523). Standard manufacturer protocols were employed. Plasmids harboring the mutant libraries of prenyltransferase genes were transformed into E. coli strain BL21(DE3) and plated onto Agar plates with suitable antibiotic selection. Active variants were identified based on the activity assay described below.

### Example 2. Cell Culture and Protein Purification for Screening Homologs and Mutant Libraries

DNA plasmids containing each of the NphB variants were individually transformed into OneShot BL21(DE3) chemically competent E. coli cells (Invitrogen catalog C600003) according to the chemically competent cell transformation protocol provided by Invitrogen. This resulted in individual E. coli cell lines, each containing one plasmid encoding a homolog variant.

To induce protein expression, individual cell lines encoding each of the “NphB variants” or prenyltransferase homologs was individually inoculated into 10 milliliters LB media with 50 micrograms per milliliter of Kanamycin sulfate in 15 milliliter culture tubes and grown at 37 degrees Celsius for 16 hours with vigorous shaking. After 16 hours, each culture was diluted into 90 milliliters LB media with 50 micrograms per milliliter of Kanamycin sulfate for a total of 100 milliliters. The absorbance at 600 nm (0D600) was monitored until it reached a value of 0.6 absorbance units. When the OD600 reached a value of 0.6, then IPTG was added to each culture to a final concentration of 500 micrograms per milliliter, resulting in an “induced culture”. Each “induced culture” was grown at 20 degrees Celsius with vigorous shaking for 20 hours.

After the cultures were grown under protein induction conditions, the target protein was extracted following a standard protein purification protocol. Each “induced culture” was spun at 4,000G for 5 minutes. The supernatant was discarded, leaving only a cell pellet. Each individual cell pellet was resuspended in 2 milliliters of a solution containing 20 millimolar Tris-HCL, 500 millimolar sodium chloride, 5 millimolar imidazole, and 10% glycerol (“lysis buffer”), resulting in a “cell slurry.” To each individual “cell slurry”, 1 microliter of 25 units per microliter Benzonase (Millipore, Benzonase, catalog number 70664-1), as well as 20 microliters of phosphatase and protease inhibitor (Thermo-Fisher, Halt Protease and Phosphatase Inhibitor Cocktail, EDTA-free, catalog number 78441) was added. Each individual “cell slurry” was then subjected to 30 second pulses of sonication, 4 times each, for a total of 120 seconds, using the Fisher Scientific Sonic Dismembrator Model 500 under 30% amplitude conditions. In between each 30 second pulse of sonication, the “cell slurry” was placed on ice for 30 seconds. After sonication, each individual “cell slurry” was centrifuged for 15 minutes at 14,000 times gravity.

Protein purification columns (Qiagen, Ni-NTA Spin Columns, catalog number 31014) were prepared by adding 600 microliters of “lysis buffer” and centrifuging at 900×g for 30 seconds. The columns were then uncapped and the resulting flow-through was discarded. 600 microliters of the supernatant from the “cell slurry” was added to the spin columns. Columns were spun at 270×g for 5 minutes. The flow through was discarded and this step was repeated until all the supernatant had been passed through the column. The resin was then washed 2 times with 600 microliters of a solution containing 20 millimolar Tris-HCl, 500 millimolar sodium chloride, and 20 millimolar imidazole (“wash buffer”) and centrifuged at 900×g for 30 seconds. The flow-through from the wash steps was discarded. The protein was then eluted off the column with 300 microliters of a solution containing 20 millimolar Tris-HCl, 200 millimolar sodium chloride, and 250 millimolar imidazole by centrifuging at 900×g for 30 seconds. This step was repeated for a final elution of 600 microliters. The eluted protein was collected and dialyzed overnight in 4 liters of a solution containing 200 millimolar Tris-HCl and 800 millimolar sodium chloride in 3.5-5.0 kilodalton dialysis tubing (Spectrum Labs, Spectra/Por dialysis tubing, catalog number 133198). After overnight dialysis, protein was concentrated to approximately 10 milligrams per milliliter using centrifugal protein filters according to manufacturer protocols (Millipore Amicon Ultra-0.5 mL Centrifugal Filter Unit, catalog number UFC500396).

### Example 3. High-Throughput Activity Assay

The library of prenyltransferase homologs and mutants were screened for protein expression by western blot with an anti-HIS antibody (Cell Signaling Technologies, anti-his monoclonal antibody, catalog number 23655) according to the protocol provided by Cell Signaling Technologies for the antibody. The variants that had detectable levels of protein expression as determined by western blot were used in a prenylation assay.

Proteins that exhibited detectable expression by Western blot were assayed for prenylation activity using a substrate (e.g., olivetolic acid, olivetol, divarinic acid, etc.) and a donor molecule (e.g., GPP, FPP, DMAPP, etc.). Unless otherwise stated, each prenylation reaction assay was performed in a volume of 20 microliters and contained 20 millimolar magnesium chloride (MgCl₂), 2 millimolar donor molecule (e.g., GPP), 100 millimolar HEPES buffer at a pH of 7.5, 2 millimolar substrate (e.g., olivetolic acid), and 20 micrograms prenyltransferase protein. These reactions were incubated for 16 hours at 30° C.

For those prenylation reactions carried out at pH 5.5, the same prenylation reaction assay protocol above was carried out with the exception of replacing HEPES buffer with sodium citrate at pH 5.5.

To assess prenylation activity by HPLC, the prenylation products were extracted from the assay reaction with the following protocol: 40 microliters of ethyl acetate was added to each reaction and vortexed thoroughly. After vortexing, each reaction was centrifuged for 10 minutes at 14,000G. The top layer (“organic layer”) was collected. This was repeated twice. The collected organic layer was evaporated, and the resulting residue was resuspended in 40 microliters of 100% methanol. After resuspending in methanol, 40 microliters of 100% HPLC grade water was added to bring the final solution to 50% methanol. These will be referred to as the “variant reactions with GPP.”

The final 50% methanol solutions were run on a Thermo Fisher UltiMate 3000 UHPLC with an Acclaim RSLC 120 angstrom C18 column with a 4 millimeter Phenomonex Securityguard guard column (54 millimeter total column length).

The wild type NphB standard prenylation assay produced two products as determined by HPLC, with retention times of approximately 6.9 minutes (an “early product” which is cannabigerolic acid [CBGA; 3-GOLA]) and 7.2 minutes (a “late product” 5-GOLA) in a ratio of ˜20% CBGA (3-GOLA) to 80% 5-GOLA.

### Example 4. Identification of Homologs and Mutant Variants

Prenyltransferase homologs (SEQ ID NOs: 1-27), found by BLAST search and rational approaches, were cloned and expressed in E. coli (as described above), and assayed in an in vitro assay for activity on OLA and GPP, using LCMS and/or HPLC to detect CBGA or 5-GOLA, as described above.

Using crystal structure of NphB (based on SEQ ID NO: 1) as a prenyltransferase template, site-saturation mutagenesis experiments were performed to identify amino acid positions that conferred improved activity towards formation of CBGA, as well as regioselectivity towards CBGA (3-GOLA) over the undesired product 5-GOLA.

Mutation at several residue positions resulted in very high regiospecificity towards CBGA, and combinatorial mutagenesis at selected residues with particular subsets of amino acids was performed to identify further unique combinatorial variants with enhanced activity and regioselectivity. Unique separate variants were identified with high activities, and with high regioselectivity for CBGA.

### Example 5. Activities with Olivetolic Acid and its Analogs

Non-natural prenyltransferases generated by engineering mutations into various wild-type prenyltransferases or modified prenyltransferases were compared to a wild-type enzyme, e.g., SEQ ID NO: 1 (NphB) for activity with either olivetolic acid or olivetolic acid analogs, divarinolic acid and orsellinic acid, and co-substrate GPP. The non-natural prenyltransferases were expressed in E. coli, purified and assayed. Assay components were as follows: each prenylation reaction assay was performed in a volume of 20 microliters and contained 20 millimolar magnesium chloride (MgCl₂), 2 millimolar donor molecule (e.g., GPP), 100 millimolar HEPES buffer at a pH of 7.5, 2 millimolar substrate (e.g., olivetolic acid), and 20 micrograms prenyltransferase protein. These reactions were incubated for 16 hours at 30° C. Products determined included cannabigerolic acid (CBGA, 3-GOLA) or its 5-GOLA isomer, cannabigerovarinic acid (CBGVA, 3-GDVA) or its 5-GDVA isomer, cannabigerorcinic acid (CBGOA, 3-GOSA) or its 5-GOSA isomer and cannabigerol (CBG, 2-GOL) or its 4-GOL isomer. The results with the acid substrates are shown in FIGS. 6A-6C.

The wild-type enzyme produced a mix of 3-GOLA and 5-GOLA. Decarboxylation of this mixture would result in a composition containing a mixture of CBG (2-GOL) with the less desired 4-GOL isomer. Select non-natural prenyltransferases were superior to wild-type enzyme in amount of desired cannabinoid produced over time, e.g., CBGA, CBGVA and CBGOA. In addition, select non-natural prenyltransferases did not produce the undesirable 5-GOLA or 3-GDVA isomers. These unique compositions provide a further advantage that subsequent steps to purify the cannabinoid can avoid an isomer separation step. Decarboxylation of the mixtures would result in a composition containing desired product CBG (2-GOL) without the less desired 4-GOL or containing desired product CBGV without its undesired isomer. Yet a further advantage is that subsequent steps to purify the decarboxylated cannabinoid can avoid an isomer separation step.

The wild-type enzyme produced a mixture of CBG and its less desired 4-GOL isomer. Except for one variant, the non-natural prenyltransferases were superior to wild-type enzyme in amount of CBG produced with olivetol as substrate. In addition, the non-natural prenyltransferases did not produce 4-GOL, providing unique compositions of product CBG.

These unique compositions provide a further advantage that subsequent steps to purify the cannabinoid can avoid an isomer separation step. In addition, the non-natural prenyltransferases provide a means to generate the desired CBG directly without the need for producing and decarboxylating an acid precursor.

### Example 6. Crystallography and Rational Enzyme Engineering of NphB and Respective Homologs

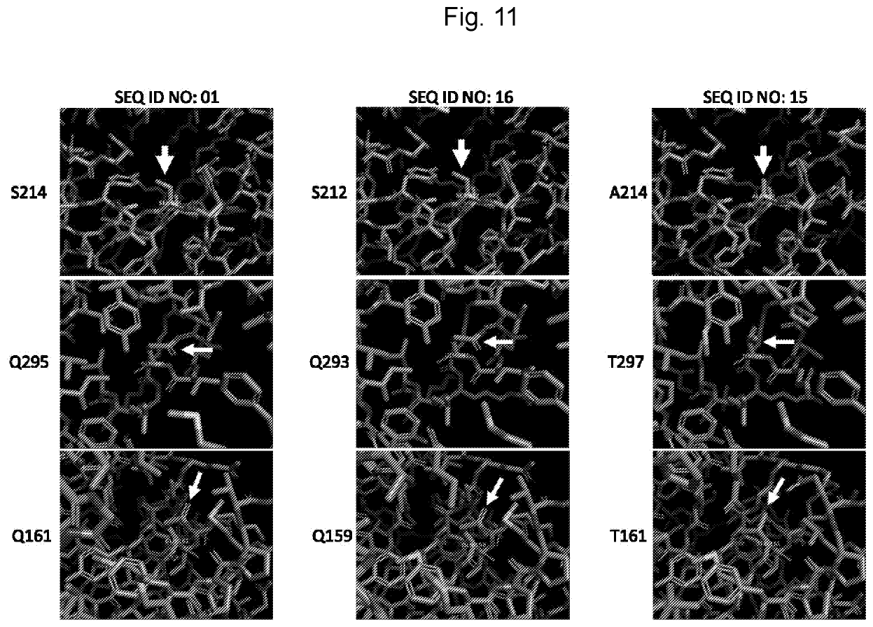

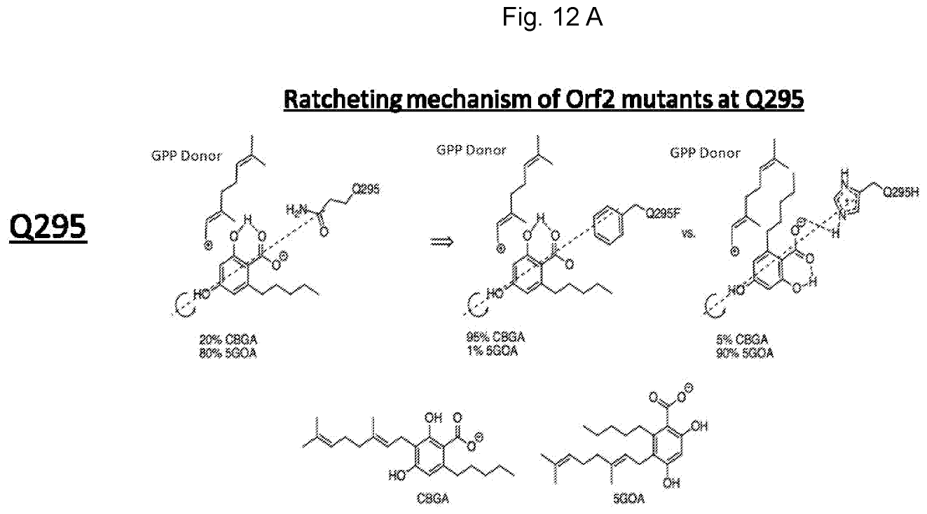

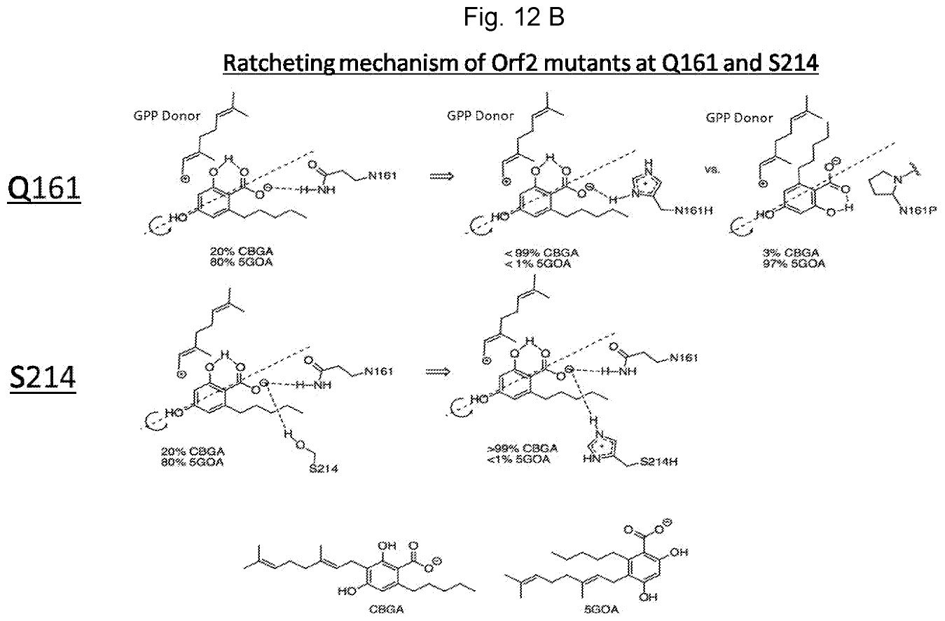

Using the crystal structure of NphB with substrates bound, one skilled in the art can observe that WT NphB enzyme contains an active site Q161 and S214 which both form a weak hydrogen bond with the carboxylate of olivetolic acid, resulting in a ˜1:5 ratio CBGA:5-GOLA. Mutagenesis at positions Q161 to Q161H, creating a more permanent hydrogen bond donor results in almost 100% CBGA production. Mutation to Q161P loses the hydrogen bond donor, as well as modifying the secondary structure at this position. Here the olivetolic acid flips its binding position within the active site, resulting in 97% 5-GOLA. Similarly, S214, which sits opposite in the pocket, can be mutated to S214H, which can also hydrogen bond to olivetolic acid carboxylate and also results in almost 100% CBGA production. Mutation to S214V also flips its binding position, resulting in 90% 5-GOLA (FIG. 12A). One skilled in the art can use sequences of homologs aligned and modeled against the crystal structure of NphB (SEQ ID NO:1) to then identify orthologous residues to Q161 and S214 from SEQ ID NO: 1, which match the relative location and orientation of those in NphB. For example, when aligned and modeled against NphB's crystal structure, one skilled in the art can identify analogous residues in SEQ ID NO: 15 and SEQ ID NO: 16 (T161 and Q159, respectively) as depicted in FIG. 11. Similarly, residues orthologous to S214 were identified in SEQ ID NO: 15 and SEQ ID NO: 16 (A214 and S212, respectively) (FIG. 11). Mutations in another homolog at an orthologous site to Q161 or S214 in SEQ ID NO: 1 may render an optimization of CBGA production and regiospecific prenylation similar to those experienced by the mutation of Q161 and S214 on SEQ ID NO: 1.

Furthermore, using the crystal structure of NphB (e.g., SEQ ID NO: 1) with substrates bound, one skilled in the art can see that Q295 in NphB is located at the entry point of the pocket for donor and substrate binding. The Q295 can interact with both the hydrocarbon tail of olivetolic acid, as well as the hydrophobic terminus of the GPP substrate. Mutation Q295 to Q295F enhances these hydrophobic interactions, leading to 98% CBGA. Alternatively mutating to Q295H forms a protonated residue, which can destabilize the hydrocarbon tail, resulting in the substrate ratcheting binding orientation. The resulting hydrogen bond with the carboxylate of olivetolic acid stabilizes the flipped binding orientation, resulting in 90% 5-GOLA (FIG. 12B). One skilled in the art can use sequences of homologs aligned and modeled against the crystal structure of NphB to then identify the orthologous residue to Q295 which matches the relative location and orientation of that in NphB (SEQ ID NO: 1). For example, when aligned and modeled against NphB's crystal structure, one skilled in the art can identify the orthologous residues in SEQ ID NO: 15 and SEQ ID NO: 16 (T297 and Q293, respectively) as depicted in FIG. 11. Mutations in another homolog at an orthologous site to Q295 in SEQ ID NO: 1 may render an optimization of CBGA production and regiospecific prenylation similar to those experienced by the mutation of Q295 on SEQ ID NO: 1.

### Example 7. Crystallography and Rational Enzyme Engineering of Homologs of NphB

The crystal structure of NphB with bound substrate demonstrates that active site residues Q161 and S214 form a weak hydrogen bond with the carboxylate of olivetolic acid which result in an approximate 1:5 ration of CBGA:5-GOLA. Mutagenesis at positions Q161 to Q161H, creating a more permanent hydrogen bond donor results in almost 100% CBGA production. Mutation to Q161P loses the hydrogen bond donor, as well as modifying the secondary structure at this position. Here the olivetolic acid flips its binding position within the active site, resulting in 97% 5-GOLA. Similarly, S214, which sits opposite in the pocket, can be mutated to S214H, which can also hydrogen bond to olivetolic acid carboxylate and also results in almost 100% CBGA production. Mutation to S214V also flips its binding position, resulting in 90% 5-GOLA (FIG. 12A). Sequences of homologs aligned and modeled against the crystal structure of NphB (SEQ ID NO:1) to then identify orthologous residues to Q161 and S214 from SEQ ID NO: 1, which match the relative location and orientation of those in NphB. Examples are shown above and in FIG. 11. Following the same rationale, select orthologous sites were identified as targets for amino acid substitutions in SEQ ID NO: 16 and SEQ ID NO: 23.

The crystal structure of NphB (e.g., SEQ ID NO: 1) with bound substrate demonstrates that Q295 in NphB is located at the entry point of the pocket for donor and substrate binding. The Q295 can interact with both the hydrocarbon tail of olivetolic acid, as well as the hydrophobic terminus of the GPP substrate. Mutation Q295 to Q295F enhances these hydrophobic interactions, leading to 98% CBGA. Alternatively mutating to Q295H forms a protonated residue, which can destabilize the hydrocarbon tail, resulting in the substrate ratcheting binding orientation. The resulting hydrogen bond with the carboxylate of olivetolic acid stabilizes the flipped binding orientation, resulting in 90% 5-GOLA (FIG. 12B).

Sequences of homologs are aligned and modeled against the crystal structure of NphB which then identified the orthologous residue to Q295, which matches the relative location and orientation in NphB (SEQ ID NO: 1). When aligned and modeled against NphB's crystal structure the orthologous residues in SEQ ID NO: 16 and SEQ ID NO: 23 (Q293, respectively for both given homologs) are identified.

Mutations in another homolog at an orthologous site to Q295 in SEQ ID NO: 1 may render an optimization of CBGA production and regiospecific prenylation similar to those experienced by the mutation of Q295 on SEQ ID NO: 1. To create non-natural variants [SEQ ID NOs: 28-53] presented in Table 3, amino acid substitutions were introduced to wild-type enzymes, (e.g., SEQ ID NO: 16 or SEQ ID NO: 23) as scaffolds.

Some non-natural variants presented in Table 2 were created on a scaffold of SEQ ID NO: 54 or SEQ ID NO: 55, which are also derived by mutagenesis from SEQ ID NO: 16 or SEQ ID NO: 23, respectively. Specifically, SEQ ID NO: 54 is identical to SEQ ID NO: 16 except for a 2-amino-acid insertion (i.e. “Gly-Ser”) at peptide sequence position 45. Similarly, SEQ ID NO: 55 is identical to SEQ ID NO: 23 except for a 2-amino-acid insertion (i.e. “Gly-Ser”) at peptide sequence position 45. Introduction of this “Gly-Ser” (“GS”) insertion was rationally conceived to mimic the respective insertion naturally found in SEQ ID NO: 1 (i.e. NphB), as seen in multiple alignment sequences displayed in FIG. 5.

### Example 8. Activities on Non-Natural Variants with Olivetolic Acid

Non-natural prenyltransferases generated by engineering mutations into wild-type prenyltransferases or modified prenyltransferases were compared to a respective wild-type enzymes, (e.g., SEQ ID NO: 16 or SEQ ID NO: 23) for activity with olivetolic acid, and co-substrate GPP. The non-natural prenyltransferases were expressed in E. coli, purified and assayed (as described above). Assay components were as follows: each prenylation reaction assay was performed in a volume of 20 microliters and contained 20 millimolar magnesium chloride (MgCl₂), 2 millimolar donor molecule (e.g., GPP), 100 millimolar HEPES buffer at a pH of 7.5, 2 millimolar substrate (e.g., olivetolic acid), and 20 micrograms prenyltransferase protein. These reactions were incubated for 16 hours at 30° C. Products determined included cannabigerolic acid (CBGA, 3-GOLA) or its 5-GOLA isomer.

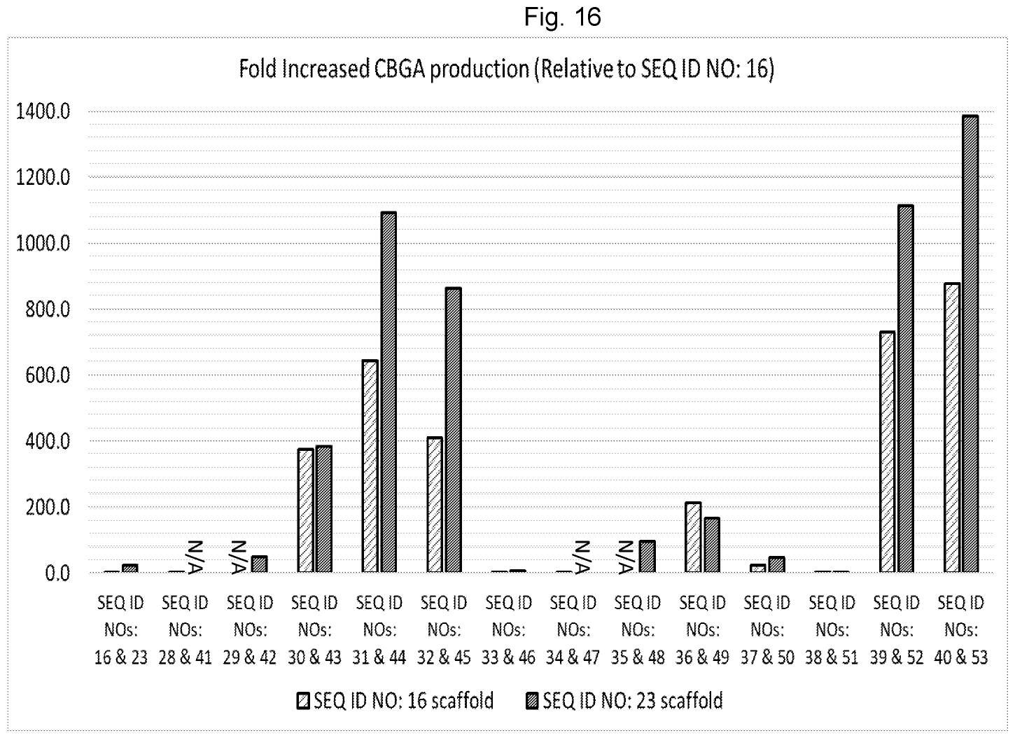

The wild-type enzyme produced a mix of 3-GOLA and 5-GOLA and 3-GOLA is reported in this example. Select non-natural prenyltransferases were superior to wild-type enzyme in amount of desired cannabinoid produced over time (e.g., CBGA). The results with the acid substrates are shown in Table 3 and FIG. 16. For example, results in FIG. 16 show significantly improved CBGA productivity in non-natural enzymes that include the combination of the substitutions Q161S+S214H+Y288V+INS45 (i.e. SEQ ID NO: 40 & SEQ ID NO: 53) when compared to wild type enzymes (SEQ ID NO: 16 and SEQ ID NO: 23, respectively).

TABLE 3
CBGA productivity in engineered non-natural prenyltransferases
Results for production of 3-GOLA (CBGA) by prenylation of olivelolic acid substrate and
GPP. Results are displayed as relative fold increase over the production of wild-type enzyme
of SEQ ID NO: 16. (Sequences in italics contain an insertion (“GS”) at site 45 (INS45),
and are derived from SEQ ID NO: 54 or SEQ ID NO: 55, with respective coordinates)

| | | SEQ ID NO: | SEQ ID NO: |
|---|---|---|---|
| SEQ ID numbers | Substitutions (Adjusted Coordinates) | 16 scaffold | 23 scaffold |
| SEQ ID NOs: 16 & 23 | Wild Type | 1.000 | 24.692 |
| SEQ ID NOs: 28 & 41 | Q161H + Q295W(includes INS45) | 0.344 | N/A |
| SEQ ID NOs: 29 & 42 | Q161S + Q295W(includes INS45) | N/A | 50.521 |
| SEQ ID NOs: 30 & 43 | Q159S + S212H | 374.468 | 383.110 |
| SEQ ID NOs: 31 & 44 | Q159S + Y286V | 642.829 | 1092.620 |
| SEQ ID NOs: 32 & 45 | S212H + Y286V | 408.580 | 862.219 |
| SEQ ID NOs: 33 & 46 | Q161S(includes INS45) | 1.133 | 7.359 |
| SEQ ID NOs: 34 & 47 | S214H(includes INS45) | 1.101 | N/A |
| SEQ ID NOs: 35 & 48 | Y288V(includes INS45) | N/A | 96.013 |
| SEQ ID NOs: 36 & 49 | Q159H + Q293W | 213.420 | 165.544 |
| SEQ ID NOs: 37 & 50 | Q161H(includes INS45) | 23.449 | 47.397 |
| SEQ ID NOs: 38 & 51 | Q295W(includes INS45) | 0.027 | 1.924 |
| SEQ ID NOs: 39 & 52 | Q159S + S212H + Y286V | 730.992 | 1113.089 |
| SEQ ID NOs: 40 & 53 | Q161S + S214H + Y288V(includes INS45) | 876.506 | 1385.987 |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A non-natural prenyltransferase comprising at least one amino acid variation as compared to a wild type prenyltransferase, and enzymatically capable of (a) at least two-fold greater rate of formation of 3-geranyl-olivetolate (3-GOLA) from geranyl pyrophosphate and olivetolic acid, as compared to the wild type prenyltransferase; (b) providing regioselectivity to 3-GOLA; or both (a) and (b).

2. The non-natural prenyltransferase of claim 1, enzymatically capable of at least five-fold greater rate of formation of 3-GOLA from geranyl pyrophosphate and olivetolic acid, as compared to the wild type prenyltransferase.

3. The non-natural prenyltransferase of claim 1 or 2, enzymatically capable of at least twenty-fold greater rate of formation of 3-GOLA from geranyl pyrophosphate and olivetolic acid, as compared to the wild type prenyltransferase.

4. The non-natural prenyltransferase of any one of the previous claims, enzymatically capable of 90% or greater regioselectivity to 3-GOLA.

5. The non-natural prenyltransferase of any one of the previous claims, enzymatically capable of 98% or greater regioselectivity to 3-GOLA.

6. A non-natural prenyltransferase comprising at least one amino acid variation as compared to a wild type prenyltransferase, and enzymatically capable of
(a1) at least two fold greater rate of formation of cannabigerovarinic acid (CBGVA) from geranyl pyrophosphate and divarinolic acid (DVA), as compared to the wild type prenyltransferase; (a2) 50% or greater regioselectivity to 3-geranyl-divarinolic acid (3-GDVA); or both (a1) and (a2); or
(b1) at least two fold greater rate of formation of cannabigerorcinic acid (CBGOA) from geranyl pyrophosphate and orsellinic acid (OSA), as compared to the wild type prenyltransferase; (b2) 50% or greater regioselectivity to 3-geranyl-orsellinate (3-GOSA); or both (b1) and (b2).

7. The non-natural prenyltransferase of claim 6, enzymatically capable of at least fifty-fold greater rate of formation of: (a) cannabigerovarinic acid (CBGVA) from geranyl pyrophosphate and divarinolic acid (DVA); or (b) cannabigerorcinic acid (CBGOA) from geranyl pyrophosphate and orsellinic acid (OSA), as compared to the wild type prenyltransferase.

8. The non-natural prenyltransferase of claim 6 or 7, enzymatically capable of at least two hundred fold greater rate of formation of: (a) cannabigerovarinic acid (CBGVA) from geranyl pyrophosphate and divarinolic acid (DVA); or (b) cannabigerorcinic acid (CBGOA) from geranyl pyrophosphate and orsellinic acid (OSA), as compared to the wild type prenyltransferase.

9. The non-natural prenyltransferase of any one of claims 6-8, enzymatically capable of 90% or greater regioselectivity to 3-geranyl-divarinolic acid (3-GDVA) or 3-geranyl-orsellinate (3-GOSA).

10. The non-natural prenyltransferase of any one of claims 6-9, enzymatically capable of 98% or greater regioselectivity to 3-geranyl-divarinolic acid (3-GDVA) or 3-geranyl-orsellinate (3-GOSA).

11. A non-natural prenyltransferase comprising at least one amino acid variation as compared to a wild type prenyltransferase, and enzymatically capable of regioselectively forming a 2-prenylated 5-alkylbenzene-1,3-diol from geranyl pyrophosphate and 5-alkylbenzene-1,3-diol.

12. The non-natural prenyltransferase of claim 11, wherein the 5-alkylbenzene-1,3-diol is olivetol and the 2-prenylated 5-alkylbenzene-1,3-diol is cannabigerol (CBG; 2-GOL).

13. The non-natural prenyltransferase of claim 11 or 12, enzymatically capable of 90% or greater regioselectivity to 2-prenylated 5-alkylbenzene-1,3-diol from geranyl pyrophosphate or cannabigerol (CBG; 2-GOL).

14. The non-natural prenyltransferase of any one of claims 11-13, enzymatically capable of 98% or greater regioselectivity to 2-prenylated 5-alkylbenzene-1,3-diol from geranyl pyrophosphate or cannabigerol (CBG; 2-GOL).

15. The non-natural prenyltransferase of any one of the previous claims, comprising at least two amino acid variations as compared to a wild type prenyltransferase.

16. The non-natural prenyltransferase of claim 15, comprising at least three, at least four, or at least five amino acid variations as compared to a wild type prenyltransferase.

17. The non-natural prenyltransferase of any one of the previous claims, having 50% or greater identity to SEQ ID NO: 1 (NphB) or to any one of SEQ ID NOs: 2-27.

18. The non-natural prenyltransferase of claim 17, having 90% or greater identity to SEQ ID NO: 1 (NphB) or to any one of SEQ ID NOs: 2-27.

19. The non-natural prenyltransferase of any one of the previous claims, wherein the at least one amino acid variation is made to the wild type prenyltransferase SEQ ID NO: 1 (NphB) or in any one of SEQ ID NOs: 2-27.

20. The non-natural prenyltransferase of any one of claims 17-19, comprising one or more amino acid variations at position(s) selected from the group consisting of: 17, 25, 38, 49, 51, 53, 106, 108, 112, 118, 119, 121, 123, 126, 161, 162, 166, 173, 174, 177, 205, 209, 213, 214, 216, 219, 227, 228, 230, 232, 234, 269, 270, 271, 274, 283, 286, 287, 288, 294, 295, 298, and 302, relative to SEQ ID NO: 1 (NphB).

21. The non-natural prenyltransferase of claim 20, comprising one or more amino acid variations at position(s) selected from the group consisting of: A17T, C25V, Q38G, V49A, V49L, V49S, S51T, A53C, A53D, A53E, A53F, A53G, A53H, A53I, A53K, A53L, A53M, A53N, A53P, A53Q, A53R, A53S, A53T, A53V, A53W, A53Y, M106E, A108G, E112D, E112G, K118N, K118Q, K119A, K119D, Y121W, F123L, F123A, F123H, F123W, T126R, Q161H, Q161R, Q161S, Q161T, Q161Y, Q161A, Q161F, Q161G, Q161I, Q161K, Q161L, Q161M, Q161C, Q161D, Q161E, Q161N, Q161P, Q161V, Q161W, M162A, M162F, D166E, N173D, L174V, S177E, S177W, S177Y, S177H, S177K, S177R, G205L, G205M, C209G, F213M, S214A, S214C, S214D, S214E, S214F, S214G, S214I, S214K, S214L, S214M, S214N, S214P, S214Q, S214R, S214T, S214V, S214W, S214Y, S214H, Y216A, L219F, D227E, R228E, R228Q, C230N, C230S, A232S, I234H, T269W, L270Y, V271E, L274V, Y283L, G286E, A287Y, Y288A, Y288F, Y288L, Y288M, Y288P, Y288T, Y288V, Y288C, Y288D, Y288E, Y288G, Y288H, Y288I, Y288K, Y288N, Y288Q, Y288R, Y288S, Y288W, V294A, V294F, V294N, Q295G, Q295K, Q295L, Q295N, Q295P, Q295R, Q295F, Q295W, Q295H, Q295C, Q295A, Q295S, Q295V, Q295D, Q295Y, Q295E, Q295I, Q295M, Q295T, L298A, L298Q, L298W, and F302K, relative to SEQ ID NO: 1 (NphB).

22. The non-natural prenyltransferase of claim 21, comprising one or more amino acid variations at position(s) selected from the group consisting of:
a) 5214H;
b) Y288V;
c) Q161H;
d) Q161R and Q295V;
e) Q161S and Q295F;
f) Q161S and Q295L;
g) Q161S and S177W;
h) Q161S and S214R;
i) Q161H and Q295V;
j) Q161H and Q295W;
k) S214R and Q295F;
l) S214R and Q295F;
m) V49A and Q295L;
n) V49A and S214R;
o) Y288I and Q295V;
p) S177W and Q295A;
q) S177W and S214R;
r) A53T and Q161S;
s) A53T and Q295A;
t) A53T and Q295F;
u) A53T and Q295W;
v) A53T and S177W;
w) A53T and S214R;
x) A53T and V294A;
y) Q161S, V294A, and Q295A;
z) Q161S, V294A, and Q295W;
aa) Q161H, Y288I, and Q295W;
bb) Q161H, Y288V, and Q295M.
cc) A53T, Q161S, and Q295A;
dd) A53T, Q161S, and Q295W;
ee) A53T, Q161S, and V294A;
ff) A53T, Q161S, and V294N;
gg) A53T, V294A, and Q295A;
hh) A53T, V294A, and Q295W;
ii) Q161 S, S214H, and Y288V;
jj) A53T, Q161S, V294A, and Q295A;
kk) A53T, Q161S, V294A, and Q295W;
ll) A53T, Q161S, V294N, and Q295A;
mm) A53T, Q161S, V294N, and Q295W;, relative to SEQ ID NO: 1 (NphB)

23. The non-natural prenyltransferase of claim 21 or 22, having identity to SEQ ID NO: 1 (NphB) in the range of 35% to 95%.

24. The non-natural prenyltransferase of any one of claims 17-19, comprising one or more amino acid variations at position(s) selected from the group consisting of: 17, 25, 38, 47, 49, 51, 104, 106, 110, 116, 117, 119, 121, 124, 159, 160, 164, 171, 172, 175, 203, 207, 211, 212, 214, 217, 225, 226, 228, 230, 232, 267, 268, 269, 272, 281, 284, 285, 286, 292, 293, 296, and 300, relative to SEQ ID NO: 16.

25. The non-natural prenyltransferase of claim 24, comprising one or more amino acid variations at position(s) selected from the group consisting of: A17T, C25V, Q38G, V47A, V47L, V47S, S49T, A51C, A51D, A51E, A51F, A51G, A51H, A51I, A51K, A51L, A51M, A51N, A51P, A51Q, A51R, A51S, A51T, A51V, A51W, A51Y, M104E, A106G, E110D, E110G, K116N, K116Q, K117A, K117D, Y119W, F121L, F121A, F121H, F121W, T124R, Q159H, Q159R, Q159S, Q159T, Q159Y, Q159A, Q159F, Q159G, Q159I, Q159K, Q159L, Q159M, Q159C, Q159D, Q159E, Q159N, Q159P, Q159V, Q159W, M160A, M160F, D164E, N171D, L172V, S175E, S175W, S175Y, S175H, S175K, S175R, G203L, G203M, C207G, F211M, S212A, S212C, S212D, S212E, S212F, S212G, S212I, S212K, S212L, S212M, S212N, S212P, S212Q, S212R, S212T, S212V, S212W, S212Y, S212H, Y214A, L217F, D225E, R226E, R226Q, C228N, C228S, A230S, I232H, T267W, L268Y, V269E, L272V, Y281L, G284E, A285Y, Y286A, Y286F, Y286L, Y286M, Y286P, Y286T, Y286V, Y286C, Y286D, Y286E, Y286G, Y286H, Y286I, Y286K, Y286N, Y286Q, Y286R, Y286S, Y286W, I292A, I292F, I292N, Q293G, Q293K, Q293L, Q293N, Q293P, Q293R, Q293F, Q293W, Q293H, Q293C, Q293A, Q293S, Q293V, Q293D, Q293Y, Q293E, Q293I, Q293M, Q293T, L296A, L296Q, L296W, and F300K, relative to SEQ ID NO: 16.

26. The non-natural prenyltransferase of claim 25, comprising at least two amino acid variations at positions selected from:
(i) Q159A, and (ii) Q293F, Q293M, Q293F, or Q293F;
(i) Q159F, and (ii) Q293F, Q293W, or Q293H;
(i) Q159G, and (ii) Q293F;
(i) Q159H, and (ii) Q293W, Q293H, Q293C, Q293A, Q293S, Q293V, Q293D, Q293Y, or Q293E;
(i) Q159I, and (ii) Q293F;
(i) Q159K, and (ii) Q293V or Q293V;
(i) Q159L, and (ii) Q293W or Q293F;
(i) Q159M, and (ii) Q293F or Q293W;
(i) Q159R, and (ii) Q293V, Q293M, or Q293T;
(i) Q159S, and (ii) Y286I;
(i) S175H, and (ii) Q293V;
(i) A51T, and (ii) Q293A;
(i) A51T, and (ii) Q293W;
(i) A51T, and (ii) I292A;
(i) S175W, and (ii) Q293A;
(i) A51T, and (ii) S175W;
(i) A51T, and (ii) Q293F;
(i) A51T, and (ii) 5212R;
(i) A51T, and (ii) Q159S;
(i) Q159S and (ii) Q293F;
(i) Q159S and (ii) Q293L;
(i) S212R and (ii) Q293F;
(i) Q159S and (ii) S212R;
(i) S175W and (ii) 5212R;
(i) V47A and (ii) 5212R;
(i) Q159S and (ii) S175W; and
(i) V47A and (ii) Q293, relative to SEQ ID NO: 16.

27. The non-natural prenyltransferase of claim 25 or 26, comprising at least three amino acid variations at positions selected from:
(i) Q159H, (ii) Y286A, and (iii) Q293F, Q293M, or Q293V;
(i) Q159H, (ii) Y286I, and (iii) Q293M or Q293V;
(i) Q159H, (ii) Y286V, and (iii) Q293F, Q293M, Q293V, or Q293W;
(i) Q159L, (ii) S175H, and (iii) Q293F;
(i) S175H, (ii), Y286V, and (iii) Q293M;
(i) S175H, (ii), Y286I, and (iii) Q293M or Q293V;
(i) Q159S, (ii) S175H, and (iii) Y286I;
(i) Q159S, (ii) S175R, and (iii) Y286V;
(i) Q159S, (ii) S175S, and (iii) Y286I;
(i) Q159S, (ii) S212H, and (iii) Y286A or Y286V;
(i) Q159S, (ii) I292A, and (iii) Q293W;
(i) A51T, (ii) Q159S, and (iii) Q293W;
(i) A51T, (ii) I292A, and (iii) Q293A;
(i) A51T, (ii) I292A, and (iii) Q293W;
(i) A51T, (ii) Q159S, and (iii) Q293A;
(i) A51T, (ii) Q159S, and (iii) I292A;
(i) A51T, (ii) Q159S, and (iii) I292N; and
(i) Q159S, (ii) I292A, and (iii) Q293A, relative to SEQ ID NO: 16.

28. The non-natural prenyltransferase of any one of claims 25-27, comprising at least four amino acid variations at positions selected from:
(i) Q159H, (ii) S175H, (iii) Y286A, and (iv) Q293V;
(i) Q159H, (ii) S175H, (iii) Y286V, and (iv) Q293M or Q293V;
(i) Q159H, (ii) S175R, (iii) Y286I, and (iv) Q293M;
(i) Q159L, (ii) S175K, (iii) Y286A, and (iv) Q293V;
(i) Q159M, (ii) S175H, (iii) Y286V, and (iv) Q293F;
(i) Q159R, (ii) S175H, (iii) Y286I, and (iv) Q293Q;
(i) Q159S, (ii) S175H, (iii) Y286V, and (iv) Q293F;
(i) Q159S, (ii) S175K, (iii) Y286V, and (iv) Q293V;
(i) Q159S, (ii) S212H, (iii) Y286V, and (iv) Q293M;
(i) A51T, (ii) Q159S, (iii) I292A, and (iv) Q293W;
(i) A51T, (ii) Q159S, (iii) I292N, and (iv) Q293W;
(i) A51T, (ii) Q159S, (iii) I292A, and (iv) Q293A;
(i) A51T, (ii) Q159S, (iii) I292N, and (iv) Q293A; and
(i) A51T, (ii) Q159S, (iii) I292N, and (iv) Q293A.
, relative to SEQ ID NO: 16

29. The non-natural prenyltransferase of any one of claims 25-28, comprising at least five amino acid variations at positions selected from:
(i) Q159H, (ii) S175R, (iii) S212H, (iv) Y286A, and (v) Q293V; and
(i) Q159R, (ii) S175R, (iii) S212H, (iv) Y286I, and (v) Q293M, relative to SEQ ID NO: 16.

30. The non-natural prenyltransferase of any one of claims 21-29, further comprising one or more amino acid variations at positions selected from:
(i) F211N, F211S, A230S, G284S, and Y286N, relative to SEQ ID NO 16; or
(ii) F213N, F213S, A232S, G286S, and Y288N, relative to SEQ ID NO: 1 (NphB).

31. The non-natural prenyltransferase of any of the previous claims, comprising one or more amino acid variations at position(s) selected from the group consisting of: 17, 25, 38, 47, 49, 51, 104, 106, 110, 116, 117, 119, 121, 124, 159, 160, 164, 171, 172, 175, 203, 207, 211, 212, 214, 217, 225, 226, 228, 230, 232, 267, 268, 269, 272, 281, 284, 285, 286, 292, 293, 296, and 300, relative to SEQ ID NO: 23.

32. The non-natural prenyltransferase of claim 31, comprising one or more amino acid variations at position(s) selected from the group consisting of: A17T, C25V, Q38G, V47A, V47L, V47S, S49T, A51C, A51D, A51E, A51F, A51G, A51H, A51I, A51K, A51L, A51M, A51N, A51P, A51Q, A51R, A51S, A51T, A51V, A51W, A51Y, M104E, A106G, E110D, E110G, K116N, K116Q, K117A, K117D, Y119W, F121L, F121A, F121H, F121W, T124R, Q159H, Q159R, Q159S, Q159T, Q159Y, Q159A, Q159F, Q159G, Q159I, Q159K, Q159L, Q159M, Q159C, Q159D, Q159E, Q159N, Q159P, Q159V, Q159W, M160A, M160F, D164E, N171D, L172V, S175E, S175W, S175Y, S175H, S175K, S175R, G203L, G203M, C207G, F211M, S212A, S212C, S212D, S212E, S212F, S212G, S212I, S212K, S212L, S212M, S212N, S212P, S212Q, S212R, S212T, S212V, S212W, S212Y, S212H, Y214A, L217F, D225E, R226E, R226Q, C228N, C228S, A230S, I232H, T267W, L268Y, V269E, L272V, Y281L, G284E, A285Y, Y286A, Y286F, Y286L, Y286M, Y286P, Y286T, Y286V, Y286C, Y286D, Y286E, Y286G, Y286H, Y286I, Y286K, Y286N, Y286Q, Y286R, Y286S, Y286W, I292A, I292F, I292N, Q293G, Q293K, Q293L, Q293N, Q293P, Q293R, Q293F, Q293W, Q293H, Q293C, Q293A, Q293S, Q293V, Q293D, Q293Y, Q293E, Q293I, Q293M, Q293T, L296A, L296Q, L296W, and F300K, relative to SEQ ID NO: 23.

33. The non-natural prenyltransferase of claim 32, comprising at least two amino acid variations at positions selected from:
(i) Q159A, and (ii) Q293F, Q293M, Q293F, or Q293F;
(i) Q159F, and (ii) Q293F, Q293W, or Q293H;
(i) Q159G, and (ii) Q293F;
(i) Q159H, and (ii) Q293W, Q293H, Q293C, Q293A, Q293S, Q293V, Q293D, Q293Y, or Q293E;
(i) Q159I, and (ii) Q293F;
(i) Q159K, and (ii) Q293V or Q293V;
(i) Q159L, and (ii) Q293W or Q293F;
(i) Q159M, and (ii) Q293F or Q293W;
(i) Q159R, and (ii) Q293V, Q293M, or Q293T;
(i) Q159S, and (ii) Y286I;
(i) S175H, and (ii) Q293V;
(i) A51T, and (ii) Q293A;
(i) A51T, and (ii) Q293W;
(i) A51T, and (ii) I292A;
(i) S175W, and (ii) Q293A;
(i) A51T, and (ii) S175W;
(i) A51T, and (ii) Q293F;
(i) A51T, and (ii) 5212R;
(i) A51T, and (ii) Q159S;
(i) Q159S and (ii) Q293F;
(i) Q159S and (ii) Q293L;
(i) S212R and (ii) Q293F;
(i) Q159S and (ii) 5212R;
(i) S175W and (ii) 5212R;
(i) V47A and (ii) 5212R;
(i) Q159S and (ii) S175W; and
(i) V47A and (ii) Q293A, relative to SEQ ID NO: 23.

34. The non-natural prenyltransferase of claim 32 or 33, comprising at least three amino acid variations at positions selected from:
(i) Q159H, (ii) Y286A, and (iii) Q293F, Q293M, or Q293V;
(i) Q159H, (ii) Y286I, and (iii) Q293M or Q293V;
(i) Q159H, (ii) Y286V, and (iii) Q293F, Q293M, Q293V, or Q293W;
(i) Q159L, (ii) S175H, and (iii) Q293F;
(i) S175H, (ii), Y286V, and (iii) Q293M;
(i) S175H, (ii), Y286I, and (iii) Q293M or Q293V;
(i) Q159S, (ii) S175H, and (iii) Y286I;
(i) Q159S, (ii) S175R, and (iii) Y286V;
(i) Q159S, (ii) S175S, and (iii) Y286I;
(i) Q159S, (ii) S212H, and (iii) Y286A or Y286V;
(i) Q159S, (ii) I292A, and (iii) Q293W;
(i) A51T, (ii) Q159S, and (iii) Q293W;
(i) A51T, (ii) I292A, and (iii) Q293A;
(i) A51T, (ii) I292A, and (iii) Q293W;
(i) A51T, (ii) Q159S, and (iii) Q293A;
(i) A51T, (ii) Q159S, and (iii) I292A;
(i) A51T, (ii) Q159S, and (iii) I292N; and
(i) Q159S, (ii) I292A, and (iii) Q293A, relative to SEQ ID NO: 23.

35. The non-natural prenyltransferase of any one of claims 31-34, comprising at least four amino acid variations at positions selected from:
(i) Q159H, (ii) S175H, (iii) Y286A, and (iv) Q293V;
(i) Q159H, (ii) S175H, (iii) Y286V, and (iv) Q293M or Q293V;
(i) Q159H, (ii) S175R, (iii) Y286I, and (iv) Q293M;
(i) Q159L, (ii) S175K, (iii) Y286A, and (iv) Q293V;
(i) Q159M, (ii) S175H, (iii) Y286V, and (iv) Q293F;
(i) Q159R, (ii) S175H, (iii) Y286I, and (iv) Q293Q;
(i) Q159S, (ii) S175H, (iii) Y286V, and (iv) Q293F;
(i) Q159S, (ii) S175K, (iii) Y286V, and (iv) Q293V;
(i) Q159S, (ii) S212H, (iii) Y286V, and (iv) Q293M;
(i) A51T, (ii) Q159S, (iii) I292A, and (iv) Q293W;
(i) A51T, (ii) Q159S, (iii) I292N, and (iv) Q293W;
(i) A51T, (ii) Q159S, (iii) I292A, and (iv) Q293A;
(i) A51T, (ii) Q159S, (iii) I292N, and (iv) Q293A; and
(i) A51T, (ii) Q159S, (iii) I292N, and (iv) Q293A, relative to SEQ ID NO: 23.

36. The non-natural prenyltransferase of any one of claims 31-35, comprising at least five amino acid variations at positions selected from:
(i) Q159H, (ii) S175R, (iii) S212H, (iv) Y286A, and (v) Q293V; and
(i) Q159R, (ii) S175R, (iii) S212H, (iv) Y286I, and (v) Q293M, relative to SEQ ID NO: 23.

37. The non-natural prenyltransferase of any one of claims 31-36, further comprising one or more amino acid variations at positions selected from:
(i) F211N, F211S, A230S, G284S, and Y286N, relative to SEQ ID NO 23.

38. The non-natural prenyltransferase of any one of the previous claims, comprising one or more amino acid variations at position(s) selected from the group consisting of: 17, 25, 38, 49, 51, 53, 106, 108, 112, 118, 119, 121, 123, 126, 161, 162, 166, 173, 174, 177, 205, 209, 213, 214, 216, 219, 227, 228, 230, 232, 234, 269, 270, 271, 274, 283, 286, 287, 288, 294, 295, 298, and 302, relative to SEQ ID NO: 54.

39. The non-natural prenyltransferase of claim 38, comprising one or more amino acid variations at position(s) selected from the group consisting of: A17T, C25V, Q38G, V49A, V49L, V49S, S51T, A53C, A53D, A53E, A53F, A53G, A53H, A53I, A53K, A53L, A53M, A53N, A53P, A53Q, A53R, A53S, A53T, A53V, A53W, A53Y, M106E, A108G, E112D, E112G, K118N, K118Q, K119A, K119D, Y121W, F123L, F123A, F123H, F123W, T126R, Q161H, Q161R, Q161S, Q161T, Q161Y, Q161A, Q161F, Q161G, Q161I, Q161K, Q161L, Q161M, Q161C, Q161D, Q161E, Q161N, Q161P, Q161V, Q161W, M162A, M162F, D166E, N173D, L174V, S177E, S177W, S177Y, S177H, S177K, S177R, G205L, G205M, C209G, F213M, S214A, S214C, S214D, S214E, S214F, S214G, S214I, S214K, S214L, S214M, S214N, S214P, S214Q, S214R, S214T, S214V, S214W, S214Y, S214H, Y216A, L219F, D227E, R228E, R228Q, C230N, C230S, A232S, I234H, T269W, L270Y, V271E, L274V, Y283L, G286E, A287Y, Y288A, Y288F, Y288L, Y288M, Y288P, Y288T, Y288V, Y288C, Y288D, Y288E, Y288G, Y288H, Y288I, Y288K, Y288N, Y288Q, Y288R, Y288S, Y288W, I294A, I294F, I294N, Q295G, Q295K, Q295L, Q295N, Q295P, Q295R, Q295F, Q295W, Q295H, Q295C, Q295A, Q295S, Q295V, Q295D, Q295Y, Q295E, Q295I, Q295M, Q295T, L298A, L298Q, L298W, and F302K, relative to SEQ ID NO: 54.

40. The non-natural prenyltransferase of claim 39, comprising at least two amino acid variations at positions selected from:
(i) Q161A, and (ii) Q295F, Q295M, Q295F, or Q295F;
(i) Q161F, and (ii) Q295F, Q295W, or Q295H;
(i) Q161G, and (ii) Q295F;
(i) Q161H, and (ii) Q295W, Q295H, Q295C, Q295A, Q295S, Q295V, Q295D, Q295Y, or Q295E;
(i) Q161I, and (ii) Q295F;
(i) Q161K, and (ii) Q295V or Q295V;
(i) Q161L, and (ii) Q295W or Q295F;
(i) Q161M, and (ii) Q295F or Q295W;
(i) Q161R, and (ii) Q295V, Q295M, or Q295T;
(i) Q161S, and (ii) Y288I;
(i) S177H, and (ii) Q295V;
(i) A53T, and (ii) Q295A;
(i) A53T, and (ii) Q295W;
(i) A53T, and (ii) I294A;
(i) S177W, and (ii) Q295A;
(i) A53T, and (ii) S177W;
(i) A53T, and (ii) Q295F;
(i) A53T, and (ii) 5214R;
(i) A53T, and (ii) Q161S;
(i) Q161S and (ii) Q295F;
(i) Q161S and (ii) Q295L;
(i) S214R and (ii) Q295F;
(i) Q161S and (ii) S214R;
(i) S177W and (ii) 5214R;
(i) V49A and (ii) 5214R;
(i) Q161S and (ii) S177W; and
(i) V49A and (ii) Q295A, relative to SEQ ID NO: 54.

41. The non-natural prenyltransferase of claim 39 or 40, comprising at least three amino acid variations at positions selected from:
(i) Q161H, (ii) Y288A, and (iii) Q295F, Q295M, or Q295V;
(i) Q161H, (ii) Y288I, and (iii) Q295M or Q295V;
(i) Q161H, (ii) Y288V, and (iii) Q295F, Q295M, Q295V, or Q295W;
(i) Q161L, (ii) S177H, and (iii) Q295F;
(i) S177H, (ii), Y288V, and (iii) Q295M;
(i) S177H, (ii), Y288I, and (iii) Q295M or Q295V;
(i) Q161S, (ii) S177H, and (iii) Y288I;
(i) Q161S, (ii) S177R, and (iii) Y288V;
(i) Q161S, (ii) S177S, and (iii) Y288I;
(i) Q161S, (ii) S214H, and (iii) Y288A or Y288V;
(i) Q161S, (ii) I294A, and (iii) Q295W;
(i) A53T, (ii) Q161S, and (iii) Q295W;
(i) A53T, (ii) I294A, and (iii) Q295A;
(i) A53T, (ii) I294A, and (iii) Q295W;
(i) A53T, (ii) Q161S, and (iii) Q295A;
(i) A53T, (ii) Q161S, and (iii) I294A;
(i) A53T, (ii) Q161S, and (iii) I294N; and
(i) Q161S, (ii) I294A, and (iii) Q295A, relative to SEQ ID NO: 54.

42. The non-natural prenyltransferase of any one of claims 38-41, comprising at least four amino acid variations at positions selected from:
(i) Q161H, (ii) S177H, (iii) Y288A, and (iv) Q295V;
(i) Q161H, (ii) S177H, (iii) Y288V, and (iv) Q295M or Q295V;
(i) Q161H, (ii) S177R, (iii) Y288I, and (iv) Q295M;
(i) Q161L, (ii) S177K, (iii) Y288A, and (iv) Q295V;
(i) Q161M, (ii) S177H, (iii) Y288V, and (iv) Q295F;
(i) Q161R, (ii) S177H, (iii) Y288I, and (iv) Q295Q;
(i) Q161S, (ii) S177H, (iii) Y288V, and (iv) Q295F;
(i) Q161S, (ii) S177K, (iii) Y288V, and (iv) Q295V;
(i) Q161S, (ii) S212H, (iii) Y288V, and (iv) Q295M;
(i) A53T, (ii) Q161S, (iii) I294A, and (iv) Q295W;
(i) A53T, (ii) Q161S, (iii) I294N, and (iv) Q295W;
(i) A53T, (ii) Q161S, (iii) I294A, and (iv) Q295A;
(i) A53T, (ii) Q161S, (iii) I294N, and (iv) Q295A; and
(i) A53T, (ii) Q161S, (iii) I294N, and (iv) Q295A, relative to SEQ ID NO: 54.

43. The non-natural prenyltransferase of any one of claims 38-42, comprising at least five amino acid variations at positions selected from:
(i) Q161H, (ii) S177R, (iii) S214H, (iv) Y288A, and (v) Q295V; and
(i) Q161R, (ii) S177R, (iii) S214H, (iv) Y288I, and (v) Q295M, relative to SEQ ID NO: 54.

44. The non-natural prenyltransferase of any one of claims 38-43, further comprising one or more amino acid variations at positions selected from:
(i) F213N, F213S, A232S, G286S, and Y288N, relative to SEQ ID NO 54.

45. The non-natural prenyltransferase of any one of the previous claims, comprising one or more amino acid variations at position(s) selected from the group consisting of: 17, 25, 38, 49, 51, 53, 106, 108, 112, 118, 119, 121, 123, 126, 161, 162, 166, 173, 174, 177, 205, 209, 213, 214, 216, 219, 227, 228, 230, 232, 234, 269, 270, 271, 274, 283, 286, 287, 288, 294, 295, 298, and 302, relative to SEQ ID NO: 55.

46. The non-natural prenyltransferase of claim 45, comprising one or more amino acid variations at position(s) selected from the group consisting of: A17T, C25V, Q38G, V49A, V49L, V49S, S51T, A53C, A53D, A53E, A53F, A53G, A53H, A53I, A53K, A53L, A53M, A53N, A53P, A53Q, A53R, A53S, A53T, A53V, A53W, A53Y, M106E, A108G, E112D, E112G, K118N, K118Q, K119A, K119D, Y121W, F123L, F123A, F123H, F123W, T126R, Q161H, Q161R, Q161S, Q161T, Q161Y, Q161A, Q161F, Q161G, Q161I, Q161K, Q161L, Q161M, Q161C, Q161D, Q161E, Q161N, Q161P, Q161V, Q161W, M162A, M162F, D166E, N173D, L174V, S177E, S177W, S177Y, S177H, S177K, S177R, G205L, G205M, C209G, F213M, S214A, S214C, S214D, S214E, S214F, S214G, S214I, S214K, S214L, S214M, S214N, S214P, S214Q, S214R, S214T, S214V, S214W, S214Y, S214H, Y216A, L219F, D227E, R228E, R228Q, C230N, C230S, A232S, I234H, T269W, L270Y, V271E, L274V, Y283L, G286E, A287Y, Y288A, Y288F, Y288L, Y288M, Y288P, Y288T, Y288V, Y288C, Y288D, Y288E, Y288G, Y288H, Y288I, Y288K, Y288N, Y288Q, Y288R, Y288S, Y288W, I294A, I294F, I294N, Q295G, Q295K, Q295L, Q295N, Q295P, Q295R, Q295F, Q295W, Q295H, Q295C, Q295A, Q295S, Q295V, Q295D, Q295Y, Q295E, Q295I, Q295M, Q295T, L298A, L298Q, L298W, and F302K, relative to SEQ ID NO: 55.

47. The non-natural prenyltransferase of claim 46, comprising at least two amino acid variations at positions selected from:
(i) Q161A, and (ii) Q295F, Q295M, Q295F, or Q295F;
(i) Q161F, and (ii) Q295F, Q295W, or Q295H;
(i) Q161G, and (ii) Q295F;
(i) Q161H, and (ii) Q295W, Q295H, Q295C, Q295A, Q295S, Q295V, Q295D, Q295Y, or Q295E;
(i) Q161I, and (ii) Q295F;
(i) Q161K, and (ii) Q295V or Q295V;
(i) Q161L, and (ii) Q295W or Q295F;
(i) Q161M, and (ii) Q295F or Q295W;
(i) Q161R, and (ii) Q295V, Q295M, or Q295T;
(i) Q161S, and (ii) Y288I;
(i) S177H, and (ii) Q295V;
(i) A53T, and (ii) Q295A;
(i) A53T, and (ii) Q295W;
(i) A53T, and (ii) I294A;
(i) S177W, and (ii) Q295A;
(i) A53T, and (ii) S177W;
(i) A53T, and (ii) Q295F;
(i) A53T, and (ii) 5214R;
(i) A53T, and (ii) Q161S;
(i) Q161S and (ii) Q295F;
(i) Q161S and (ii) Q295L;
(i) S214R and (ii) Q295F;
(i) Q161S and (ii) 5214R;
(i) S177W and (ii) 5214R;
(i) V49A and (ii) 5214R;
(i) Q161S and (ii) S177W; and
(i) V49A and (ii) Q295A, relative to SEQ ID NO: 55.

48. The non-natural prenyltransferase of claim 46 or 47, comprising at least three amino acid variations at positions selected from:
(i) Q161H, (ii) Y288A, and (iii) Q295F, Q295M, or Q295V;
(i) Q161H, (ii) Y288I, and (iii) Q295M or Q295V;
(i) Q161H, (ii) Y288V, and (iii) Q295F, Q295M, Q295V, or Q295W;
(i) Q161L, (ii) S177H, and (iii) Q295F;
(i) S177H, (ii), Y288V, and (iii) Q295M;
(i) S177H, (ii), Y288I, and (iii) Q295M or Q295V;
(i) Q161S, (ii) S177H, and (iii) Y288I;
(i) Q161S, (ii) S177R, and (iii) Y288V;
(i) Q161S, (ii) S177S, and (iii) Y288I;
(i) Q161S, (ii) S214H, and (iii) Y288A or Y288V;
(i) Q161S, (ii) I294A, and (iii) Q295W;
(i) A53T, (ii) Q161S, and (iii) Q295W;
(i) A53T, (ii) I294A, and (iii) Q295A;
(i) A53T, (ii) I294A, and (iii) Q295W;
(i) A53T, (ii) Q161S, and (iii) Q295A;
(i) A53T, (ii) Q161S, and (iii) I294A;
(i) A53T, (ii) Q161S, and (iii) I294N; and
(i) Q161S, (ii) I294A, and (iii) Q295A, relative to SEQ ID NO: 55.

49. The non-natural prenyltransferase of any one of claims 45-48, comprising at least four amino acid variations at positions selected from:
(i) Q161H, (ii) S177H, (iii) Y288A, and (iv) Q295V;
(i) Q161H, (ii) S177H, (iii) Y288V, and (iv) Q295M or Q295V;
(i) Q161H, (ii) S177R, (iii) Y288I, and (iv) Q295M;
(i) Q161L, (ii) S177K, (iii) Y288A, and (iv) Q295V;
(i) Q161M, (ii) S177H, (iii) Y288V, and (iv) Q295F;
(i) Q161R, (ii) S177H, (iii) Y288I, and (iv) Q295Q;
(i) Q161S, (ii) S177H, (iii) Y288V, and (iv) Q295F;
(i) Q161S, (ii) S177K, (iii) Y288V, and (iv) Q295V;
(i) Q161S, (ii) S212H, (iii) Y288V, and (iv) Q295M;
(i) A53T, (ii) Q161S, (iii) I294A, and (iv) Q295W;
(i) A53T, (ii) Q161S, (iii) I294N, and (iv) Q295W;
(i) A53T, (ii) Q161S, (iii) I294A, and (iv) Q295A;
(i) A53T, (ii) Q161S, (iii) I294N, and (iv) Q295A; and
(i) A53T, (ii) Q161S, (iii) I294N, and (iv) Q295A, relative to SEQ ID NO: 55.

50. The non-natural prenyltransferase of any one of claims 45-49, comprising at least five amino acid variations at positions selected from:
(i) Q161H, (ii) S177R, (iii) S214H, (iv) Y288A, and (v) Q295V; and
(i) Q161R, (ii) S177R, (iii) S214H, (iv) Y288I, and (v) Q295M, relative to SEQ ID NO: 55.

51. The non-natural prenyltransferase of any one of claims 45-50, further comprising one or more amino acid variations at positions selected from:
(i) F213N, F213S, A232S, G286S, and Y288N, relative to SEQ ID NO 55.

52. A nucleic acid encoding the non-natural prenyltransferase of any one of the previous claims.

53. An expression construct comprising the nucleic acid of claim 52.

54. An engineered cell comprising a non-natural prenyltransferase comprising at least one amino acid variation as compared to a wild type prenyltransferase, and enzymatically capable of (a) at least two fold greater rate of formation of cannabigerolic acid (CBGA) from geranyl pyrophosphate and olivetolic acid, as compared to the wild type prenyltransferase; (b) 50% or greater regioselectivity to 3-geranyl-olivetolate (3-GOLA); or both (a) and (b).

55. An engineered cell comprising a non-natural prenyltransferase comprising at least one amino acid variation as compared to a wild type prenyltransferase, and enzymatically capable of:
a1) at least two fold greater rate of formation of cannabigerovarinic acid (CBGVA) from geranyl pyrophosphate and divarinolic acid (DVA), as compared to the wild type prenyltransferase; (a2) 50% or greater regioselectivity to 3-geranyl-divarinolic acid (3-GDVA); or both (a1) and (a2); or
(b1) at least two fold greater rate of formation of cannabigerorcinic acid (CBGOA) from geranyl pyrophosphate and orsellinic acid (OS A), as compared to the wild type prenyltransferase; (b2) 50% or greater regioselectivity to 3-geranyl-orsellinate (3-GOSA); or both (b1) and (b2).

56. An engineered cell comprising a non-natural prenyltransferase comprising at least one amino acid variation as compared to a wild type prenyltransferase, and enzymatically capable of regioselectively forming a 2-prenylated 5-alkylbenzene-1,3-diol from geranyl pyrophosphate and 5-alkylbenzene-1,3-diol.

57. The engineered cell of claim 56, wherein the 5-alkylbenzene-1,3-diol is olivetol and the prenylated alcohol 2-prenylated 5-alkylbenzene-1,3-diol is cannabigerol (CBG; 2-GOL).

58. The engineered cell of any one of claims 54-57, comprising a non-natural prenyltransferase of any one of claims 1-51 or nucleic acid of claim 52 or 53.

59. The engineered cell of claim 54 or 58, comprising an olivetolic acid pathway.

60. The engineered cell of claim 59, wherein the olivetolic acid pathway comprises polyketide synthase/olivetol synthase (condensation of hexanoyl coenzyme A (CoA) and malonyl CoA) along with olivetolic acid cyclase (OAC).

61. The engineered cell of claim 55 or 58, comprising a DVA or OSA pathway.

62. The engineered cell of any one of claims 56-58, comprising an olivetol pathway.

63. The engineered cell of claim 62, wherein the olivetol pathway comprises polyketide synthase.

64. The engineered cell of any one of claims 54-63, comprising a geranyl pyrophosphate pathway.

65. The engineered cell of claim 64, wherein the geranyl pyrophosphate (GPP) pathway comprises geranyl pyrophosphate synthase.

66. The engineered cell of claim 65, wherein the GPP pathway comprises a mevalonate (MVA) pathway, a MEP pathway, or both.

67. The engineered cell of any one of claims 54-66, comprising two or more exogenous nucleic acids, wherein one of the two or more exogenous nucleic acids encodes the non-natural prenyltransferase.

68. The engineered cell of claim 67, wherein the exogenous nucleic acids encodes an enzyme in (a) the olivetolic acid pathway, (b) the geranyl pyrophosphate pathway, or both (a) and (b).

69. The engineered cell of claim 67, wherein the exogenous nucleic acids encodes an enzyme in (a) the DVA or OSA pathway, (b) the geranyl pyrophosphate pathway, or both (a) and (b).

70. The engineered cell of claim 67, wherein the exogenous nucleic acids encodes an enzyme in (a) the olivetol pathway, (b) the geranyl pyrophosphate pathway, or both (a) and (b).

71. The engineered cell of any one of claims 54-50, selected from the group consisting of yeast, microalgae, Escherichia, Corynebacterium, Bacillus, Ralstonia, and Staphylococcus.

72. A cell extract or cell culture medium comprising cannabigerolic acid (CBGA) derived from the engineered cell of any one of claim 54, 58-60, or 63-68.

73. A cell extract or cell culture medium of claim 72, comprising cannabigerolic acid (CBGA) at 50% or greater of the total geranyl olivetolate (3-GOLA plus 5-GOLA) or comprising CBG at 50% or greater of the total CBG (2-GOL) plus 4-GOL.

74. A purified cannabigerolic acid (CBGA) or CBG derived from the engineered cell of any one of claim 54, 58-60, or 63-68, or the cell extract or cell culture medium of claim 72 or 73.

75. The purified cannabigerolic acid (CBGA) or CBG of claim 74, comprising cannabigerolic acid (CBGA) at 50% or greater of the total geranyl olivetolate (3-GOLA plus 5-GOLA) or comprising CBG at 50% or greater of the total CBG (2-GOL) plus 4-GOL.

76. A cell extract or cell culture medium comprising CBGVA or CBGOA derived from the engineered cell of any one of claim 55, 58-61, or 64-69.

77. A purified CBGVA or CBGOA derived from the engineered cell of any one of claim 55, 58-61, or 64-69, or the cell extract or cell culture medium of claim 76.

78. A cell extract or cell culture medium comprising cannabigerol (CBG) derived from the engineered cell of any one of claim 56-58, 62-67, or 69.

79. A purified cannabigerol (CBG) derived from the engineered cell of any one of claim 56-58, 62-67, or 69, or the cell extract or cell culture medium of claim 78.

80. A method for forming a prenylated aromatic compound, comprising contacting a hydrophobic substrate and an aromatic substrate with a non-natural prenyltransferase of any one of claims 1-51, wherein contacting forms a prenylated aromatic compound.

81. The method of claim 80, wherein the aromatic substrate is selected from the group consisting of olivetol, olivetolic acid, divarinol, divarinolic acid, orcinol, and orsellinic acid.

82. The method of claim 80 or 81, wherein the hydrophobic substrate includes any one of an isoprenoid portion, a geranyl portion, a farnesyl portion, and one or more phosphate groups.

83. The method of any one of claims 80-82, wherein the contacting occurs in the engineered cell of any one of claims 54-71.

84. The method of any one of claims 80-83, further comprising isolating or purifying the prenylated aromatic compound, or a derivative thereof, from other material.

85. The method of claim 84, wherein the isolating or purifying comprises one or more of continuous liquid-liquid extraction, pervaporation, evaporation, filtration, membrane filtration (including reverse osmosis, nanofiltration, ultrafiltration, and microfiltration), membrane filtration with diafiltration, membrane separation, reverse osmosis, electrodialysis, distillation, extractive distillation, reactive distillation, azeotropic distillation, crystallization and recrystallization, centrifugation, extractive filtration, ion exchange chromatography, size exclusion chromatography, adsorption chromatography, carbon adsorption, hydrogenation, and ultrafiltration.

86. A method of making a therapeutic composition including geranyl olivetolate, or a derivative thereof, comprising including the geranyl olivetolate, or a derivative thereof, obtained from the engineered cell of any one of claim 54, 58-60, or 63-68, or the method of any one of claims 79-85, in a therapeutic composition.

87. A therapeutic or a medicinal composition including cannabigerolic acid (CBGA), or a derivative thereof, obtained from the engineered cell of any of claim 54, 58-60, or 63-68, or the method of any of claims 80-85.

88. The therapeutic or medicinal composition of claim 87, where the derivative thereof is CBG.

89. The therapeutic or medicinal composition of claim 87 or 88, comprising CBGA or CBG at 60% or greater, 70% or greater, 80% or greater, 85% or greater, 90% or greater, 91% or greater, 92% or greater, 93% or greater, 94% or greater, 95% or greater, 96% or greater, 97% or greater, 98% or greater, 99% or greater, 99.2% or greater, 99.4% or greater, 99.5% or greater, 99.6% or greater, 99.7% or greater, 99.8% or greater, or 99.9% or greater of total cannabinoid compound(s) in the therapeutic composition.

90. A method of making a therapeutic composition including CBGVA or CBGOA, or a derivative thereof, comprising including the CBGVA or CBGOA, or a derivative thereof, obtained from the engineered cell of any one of claim 55, 58-61, or 64-69, or the method of any one of claims 80-85, in a therapeutic composition.

91. A therapeutic or a medicinal composition including CBGVA or CBGOA, or a derivative thereof, obtained from the engineered cell of any one of claim 55, 58-61, or 64-69, or the method of any one of claims 80-85.

92. The therapeutic or medicinal composition of claim 91, comprising CBGVA or CBGOA at 60% or greater, 70% or greater, 80% or greater, 85% or greater, 90% or greater, 91% or greater, 92% or greater, 93% or greater, 94% or greater, 95% or greater, 96% or greater, 97% or greater, 98% or greater, 99% or greater, 99.2% or greater, 99.4% or greater, 99.5% or greater, 99.6% or greater, 99.7% or greater, 99.8% or greater, or 99.9% or greater of total cannabinoid compound(s) in the therapeutic composition.

93. A method of making a therapeutic composition including CBG, comprising including the CBG obtained from the engineered cell of any one of claim 56-58, 62-67, or 69, or the method of any one of claims 70-85, in a therapeutic composition.

94. A therapeutic or a medicinal composition including CBG obtained from the engineered cell of any one of claims 56-58, 62-67, or the method of any one of claims 80-85.

95. The therapeutic or medicinal composition of claim 94, comprising CBG at 60% or greater, 70% or greater, 80% or greater, 85% or greater, 90% or greater, 91% or greater, 92% or greater, 93% or greater, 94% or greater, 95% or greater, 96% or greater, 97% or greater, 98% or greater, 99% or greater, 99.2% or greater, 99.4% or greater, 99.5% or greater, 99.6% or greater, 99.7% or greater, 99.8% or greater, or 99.9% or greater of total cannabinoid compound(s) in the therapeutic composition.


SEQUENCE LISTING (55 sequences)

SEQ ID NO: 1 - protein, 307 aa, Streptomyces sp. [sequence omitted]

SEQ ID NO: 2 - protein, 305 aa, Streptomyces sp.
MSGAADVERVYSAMEEAARLLDITVSREKVRPALEAYHEVLADAVVVFSMASGRYATELDFSVSVPAEAGDPYRVALAKGLTPRTGHPVGSLLADTQEHCPVSMFAFDGEITGGFKKTYAFFPTNDLPSASKLAGIPSMPDSVKENAGLFARYGLDKVQMTSIDYNKKTVNLYFSEMSPDILGPEAVRSMIRDMGLTETGEVGLTFARRSFAVYPTLNWESGRIDRLCFAVISRDPTLTPAEREEDLAKFSKYANNAPYAYAGEARTLVYGLTLTPREEYYKLGSYYQISDTQRKLLKAFDSLKD

SEQ ID NO: 3 - protein, 305 aa, Actinobacteria bacterium
MSGAADVERVYAAMEEAAGLLDVSCAREKIYPLLTVFQDTLTDGVVVFSMASGRRSTELDFSISVPVSQGDPYATVVREGLFRATGSPVDELLADTVKHLPVSMFAIDGEVTGGFKKTYAFFPTDDMPGVAQLTGIPSMPASVAENAELFARYGLDKVQMTSMDYKKRQVNLYFSDLKQEYLQPEAVVALARELGLQVPGELGLEFCKRSFAVYPTLNWDTGKIDRLCFAAISTDPTLVPSTDERDIEMFREYATKAPYAYVGEKRTLVYGLTLSPTEEYYKLGAYYHITDIQRQLLKAFDALED

SEQ ID NO: 4 - protein, 305 aa, Streptomyces tendae
MSGAADVERVYAAMEEAAGLLDVSCAREKIYPLLTVFQDTLTDGVVVFSMASGRRSTELDFSISVPVSQGDPYATVVKEGLFRATGSPVDELLADTVKHLPVSMFAIDGEVTGGFKKTYAFFPTDDMPGVAQLTEIPSMPASVAENAELFARYGLDKVQMTSMDYKKRQVNLYFSDLKQEYLQPEAVVALARELGLQVPGELGLEFCKRSFAVYPTLNWDTGKIDRLCFAAISTDPTLVPSTDERDIEMFREYATKAPYAYVGEKRTLVYGLTLSSTEEYYKLGAYYHITDIQRQLLKAFDALED

SEQ ID NO: 5 - protein, 305 aa, Streptomyces sp.
MSGAADVERVYAAMEEAAGLLDVSCAREKIYPLLTVFQDTLTDGVVVFSMASGRRSTELDFSISVPVSQGDPYATVVKEGLFQATGSPVDELLADTVAHLPVSMFAIDGEVTGGFKKTYAFFPTDDMPGVAQLAAIPSMPASVAENAELFARYGLDKVQMTSMDYKKRQVNLYFSDLKQEYLQPESVVALARELGLRVPGELGLEFCKRSFAVYPTLNWDTGKIDRLCFAAISTDPTLVPSEDERDIEMFRNYATKAPYAYVGEKRTLVYGLTLSSTEEYYKLGAYYHITDIQRQLLKAFDALED

SEQ ID NO: 6 - protein, 305 aa, Streptomyces paucisporeus
MSGAAEVERVYSAMEEAAGLLDVACSPEKVRPILTAFQDVLSDGVIVYSMASGRHATELDFSISVPADHGDPYTAALAHGLIPETDHPVGNLLADTQKALPVSMFAVDGEVTGGFKKTYAFFPTDDMPGLAQLIDIPSMPPSVAENAELFARYGLDKVQMTSLDYKRKQVNLYFSNLQPEFLAPEPVLSMVREMGLELPGEKGLKFARRSFAIYPTLGWESGKIERLCFAVISTDPGLVPAPDEADRALFSTYANNAPYAYAGEKRTLVYGLTLSPTEEYYKLGSYYQITDIQRTLLKAFDALTD

SEQ ID NO: 7 - protein, 305 aa, Streptomyces sp.
MSGAAEVERVYSAMEESAGLLDVACSREKIQPILTAFQDVLADGVIVFSMANGRHATELDFSISVPAGHGDPYAAALEHGLIPATGHPVGDLLADTQKALPVSMFAVDGEVTSGFKKTYAFFPTDDMPGLAQLIDIPSMPPSVAENAELFGRYGLDKVQMISLDYKKNQVNLYFSNLNPEFLQPEPVQAMVREMGLQLPADKGLAFAKRSFAVYPTLSWDSAKIERLCFAVISTDPTLAPAQEQADLDLFSTYANNAPYAYAGEKRTLVYGLTLSPSEEYYKLGSYYQISDIQRKLLKAFDALTD

SEQ ID NO: 8 - protein, 305 aa, Streptomyces sp.
MSGAADVERVYSAMEEAARLLDITVSREKVRPALEAYHEVLADAVVVFSMASGRYATELDFSISVPAEAGDPYRVALAKGLTPRTDHPVGRLLADTQEHCPVSMFAFDGEITGGFKKTYAFFPTNDLQSASKLAEIPSMPDSVKENADLFARYGLDKVQMTSIDYKKKAVNLYFSEMSPDILGPDTVRSMLRDMGLKETGETGLTFARRSFSVYPTLNWETGRIERLCFAVISRDPTLAPAERAEDLAKFSKYANNAPYAYAGEARTLVYGLTLTPREEYYKLGSYYQISDIQRKLLKAFDSLND

SEQ ID NO: 9 - protein, 304 aa, Unknown
  Description of Unknown ORF sequence
MSGAKDVERVYSAMEEAAGLLNVPVARDKIWPVLTAYQDALADAVIVFSMAGGRRSTELDFSISVPTDHGDPFTTALERGLTEKENHPVDNLLAELRDGFPLGMYAIDGMVTTGFKKAYASFPTNEPQPLTALLDLPSMPESARANAELFARYGLDKVQMVSVDYPKRQVNLYFSELKADHLTPEQVKATASEMGLVEPTDMALDFATGSFAVYPTLGYDSDVVDRITYAVISVDPTLAPTTSEPEKTQITTYANSAPYAYAGENRTLVYGFTLTSKEEYYKLGSYYQITDLQRTLVKAFEALD

SEQ ID NO: 10 - protein, 304 aa, Streptomyces sp.
MSGAKDVERVYSAMEEAAGLLNVPVARDKIWPVLTAYQDALADAVIVFSMAGGRRSTELDFSISVPTDHGDPFTTALERGLTEKENHPVDNLLAELRDGFPLGMYAIDGMVTTGFKKAYASFPTNEPQPLTALLDLPSMPESARANAELFARYGLDKVQMVSVDYPKRQVNLYFSDLNADHLTPEEVKSTASEMGLVEPTDMALDFATGSFAVYPTLGYDSDVVDRITYAVISVDPTLAPTTSEPEKTQITTYANSAPYAYAGENRTLVYGFTLTSKEEYYKLGSYYQITDLQRTLVKAFEALD

SEQ ID NO: 11 - protein, 304 aa, Unknown
  Description of Unknown ORF sequence
MSGANDVERVYSAMEEAAGLLNVPVARDKIWPVLTAYQDALADAVVVFSMAGGRRATELDFSISVPTDLGDPFTTALRRGLTEKTNHPVDNLLAELTDGFEIGMYAIDGMVTTGFKKTYASFPTNEPQPLTALLDVPSMPESARANAELFARYGLDKVQMVSVDYPKRQVNLYFSELDTDYLQPEHVKSLARETGLVEPTEMGLDFASGSFAVYPTLGYDNDIVDRITYAVISVDPTLAPTKSEPEVSQLSRYATSAPYAYAGENRTLVYGVTLTSKEEYYKLGSYYQITDLQRTLVKAFEALD

SEQ ID NO: 12 - protein, 304 aa, Streptomyces sp.
MSGANDVERVYSAMEEAAGLLGVPVAREKVRPVLTAYQDALADAVVVFSMAGGRRATELDFSISVPTDHGDPFTTALQRGLTEKTGHPVDNLLAELREGFPLGMYAIDGMVSTGFKKTYASFPTNEPQPLDDLLDVPSMPASARANAKLFANYGLDKVQMVSVDYPKRQVNLYFSELNTDYLQPAQVKALAAEMGLIEPSELGLEFAKGSFAVYPTLSYDTDASDRLCLAVISSDPTLAPTTSEPEVTQFSTYANNAPYAYAGENRTLVYGLTLTPKEEYYKLGSYYQITDYQRKLVKAFEALD

SEQ ID NO: 13 - protein, 306 aa, Streptomyces sp.
MSKATEVDRVYAAVEKAAALAGTTCAGDKVRPVLTGHQDLLDEAVIVFSMTASGSHSGGLDLSMTVPAEHVDPYSFALSEGLIEPTDHPVGSVISDFQERFPIGMYGIDVDVAGGFKKAYAAFPSNDLRELKQLFDLPSMPSAAAENAELFARYGLDRVTGVSVDYKRHELNLYCDRATTEPLDPDYVQSMLRDMGLKEASEQGLEFAKKTFAIYPTLNWDSSEIVRICFAVITTDPATTPTRSEPELGQMWEYANTAPYAYVGEQRALVYGLALSPEKEYYKLGAYYQISDYQRKLVKAFDALPE

SEQ ID NO: 14 - protein, 305 aa, Streptomyces niveus
MSGAADVERVYSAMERAAGLLDLTCAREKILPILTAYKEALADSVIVFSMSGGDHSAELDFSFTIPSGDVDPYAFGPSTGIPTETDHPIASLLSDTGERCPVAMYGVDGEVSGGFKKTYAAFPINDLLDLSKLVAVPSMPPAVAENAELFARYGLDKVQGISIDYQRKQVNLYCGDIPAESLEPETVRSMLREMGLREPSEEGLEFVRKSFAVYPTLSWDSSRIERICFAVISTDPTLAPTRVESDVALFSKYANNAPYAYAGERRTLIYGLAVSPTKEYIKLGSYYQISDHQRKLVKAFDALED

SEQ ID NO: 15 - protein, 311 aa, Streptomyces sp.
MYGGTEVEEVYSALEKSAGLVGVPCNRDKVWPALSTYQDALGEAVIVFSVATDERHAGELDYTITVPTGGADPYALALAKGLTPETDHPVGTLLAGVQERCPVAGYAVDCGVVGGFKKIYSFFPQDDLQGLAKLAEIPSMPRALAENAALFARHGLDHKVTMLGIDYQRESVNLYFGKLPEECLQPDSIRAILRDIGLPEPTEPMLEFARKSFAIYVTLSWDAAKVERICFAVPPGRDLITLDPSALPARIAPEIEHFARNSPYAYPGDRMLVYGVTWSPEEEYYKLGSYYQLPVQTRKLLVAFDSVKDQE

SEQ ID NO: 16 - protein, 305 aa, Streptomyces antibioticus
MSGAADVERVYAAMEEAAGLLGVTCAREKIYPLLTEFQDTLTDGVVVFSMASGRRSTELDFSISVPTSQGDPYATVVDKGLFPATGHPVDDLLADTQKHLPVSMFAIDGEVTGGFKKTYAFFPTDDMPGVAQLSAIPSMPSSVAENAELFARYGLDKVQMTSMDYKKRQVNLYFSELSEQTLAPESVLALVRELGLHVPTELGLEFCKRSFSVYPTLNWDTGKIDRLCFAVISTDPTLVPSTDERDIEQFRHYGTKAPYAYVGENRTLVYGLTLSPTEEYYKLGAYYHITDIQRRLLKAFDALED

SEQ ID NO: 17 - protein, 308 aa, Actinopolyspora sp.
MSGDADLKEDVYSAIEKSAGLMEAPCARHEVWPILTAFGEDLGEAGIVFSVQTGERHAGELDYTITVPAGGDDPYALALSNGLLEETDHPVSNLLSDVRARCRISEYFIDCGVVGGFNKAYAHFPHDSQSVARLAEVPSMPRGLADNADFFARHGLDQVAMMGVDYGKKSVNLYFAQLSEDCLERSNILSMFRASGLPEPGERSLNFARGAFRIYVTLTWDSSEVKRIAFASLTGQEWLDSALSEFPVRVSPEIERFVRNAPYTYSGDPLRILAVKFAPEGEYLNFGSYYQISPLVRNLLAASADERS

SEQ ID NO: 18 - protein, 296 aa, Actinomadura sp.
MSETAEVKELCAVIEECARMLDVPFARPRVSSVLNAYEDAFGHAATVVAFRVATGVRHVGELDCRFTTHPDDRDPYASALAKGLTPETDHPVGDLLSDVQARCPIDSHGIDFGLVGGFKKVYAFFTPDDLQDLSKLTAMPAMPRALADNAGFLARHGLDDRVGVIGIDYRSRTVNVYFNEVPDACFEPETIRSTLREIGTAEPSERMLRLGRESFGLYVTLSWDSPEIERICFAVTTTDLATLPVRIEPEIERFVKSVPFGGDDRKFVYGVALAPEGEYYKLESHYRWKPGAMDFI

SEQ ID NO: 19 - protein, 303 aa, Streptomyces iakyrus
MEGEMSEASELAVIYSAIEETAQLLDVPCSRDKVQPALAAFGDGLTDAHIVFSMATGERYKGELAFDFTVPTAAGDPYAIALANGLVDETDHPIRSLFSDVQERCPVDSYGVDYGLVGGFKKTYVSFPLGDLQGLSTLVDVPSMPRALAEHADFFASHGLDDKVSAIAIDYAHRTWNVYFSGIPAEVKEPQTLRSVLQRFGLPEPSERLMEFIRTSFAMYTTFGWDSTKAERICFSARSSDPMALPAQFEPQIAKFAKSAPYTYTGERVLTYAGALSPSEEFYKLASFYQKTSKLSDRVRPAT

SEQ ID NO: 20 - protein, 304 aa, Streptomyces sp.
MAGANDVERVYSAMEEAAGLLGVPVAREKVRPVLTAYQDVLADAVVVFSMAGGRRATELDFSISVPTDHGDPFTTALRRGLTEKTGHPVDNLLAELREGFPLGMYAIDGMVSTGFKKTYASFPTNEPQPLADLLDVPSMPASARANAKLFANYGLDKVQMVSVDYPKRQVNLYFSELDTDYLQPAQVKALAAEMGLIEPSELGLEFAKGSFAVYPTLSYDSDAGDRLCLAVISSDPTLAPTTSEPEVTQFSTYANNAPYAYAGENRTLVYGLTLTPQEEYYKLGSYYQITDYQRKLVKAFEALD

SEQ ID NO: 21 - protein, 296 aa, Micromonospora purpureochromogenes
MSGTPEVAELYSTIEESARQLDVPCSRDRVWPILSAYGDAFAHPEAVVAFRVATALRHAGELDCRFRTHPDDRDPYASAIDRGLTPRTDHPIGGLLAEVHRRCPVESHGIDFGVVGGFKKIYAAFAPDELQVASALAGIPAMPRSLAANADFFTRHGLDDRVGVLGFDYPARTVNVYFNDVPRECFEPETIRSTLRRTGMAEPSEQMLRLGAGAFGLYVTLGWDSPEIERICYAAATTDLTTLPVPVEPEIEKFVKSVPYGGGDRKFVYGVALTPKGEYYKLESHYKWKPGAVNFI

SEQ ID NO: 22 - protein, 298 aa, Streptomyces anulatus
MSESAELTELYSAIEETTRVVGAPCRRDTVRPILTAYEDVIAQSVISFRVQTGTSDAGDLDCRFTLLPKDMDPYATALSNGLTAKTDHPVGSLLEEVHRQFPVDCYGIDFGAVGGFKKAWSFFRPDSLQSASDLAALPSMPSGVSENLGLFDRYGMTDTVSVVGFDYAKRSVNLYFTGASPESFEPRGIQAILRECGLPEPSDELLRFGEEAFAIYVTLSWDSQKIERVTYSVNTPDPMALPVRIDTRIEQLVKDAPLGSAGHRYVYGVTATPKGEYHKIQKYFQWQSRVEKMLTADA

SEQ ID NO: 23 - protein, 305 aa, Streptomyces sp.
MSGAADVERVYAAMEEAAGLLGVTCAREKIYPLLTEFQDTLTDGVVVFSMASGRRSTELDFSISVPTSQGDPYATVVEKGLFPATGHPVDDLLADTQKHLPVSMFAIDGEVTGGFKKTYAFFPTDDMPGVAQLSAIPSMPSSVAENAELFARYGLDKVQMTSMDYKKRQVNLYFSELSEQTLAPESVLALVRELGLHVPTELGLEFCKRSFSVYPTLNWDTGKIDRLCFAVISTDPTLVPSTDERDIEQFRAYGTKAPYAYVGEKRTLVYGLTLSPTEEYYKLGAYYHITDIQRRLLKAFDALED

SEQ ID NO: 24 - protein, 304 aa, Streptomyces sp.
MSGANDVERVYSAMEEAAGLLGVPVAREKVRPVLTAYQEALADAVVVFSMAGGRRATELDFSISVPTDHGDPFTTALQRGLTEKTGHPVDNLLAELREGFPLGMYAIDGMVSTGFKKTYASFPTNEPQPLADLLDVPSMPASARANAKLFADYGLDKVQMVSVDYPKRQVNLYFSELNTDYLQPAQVKALAAEMGLVEPTGMGLEFAKGSFAVYPTLSYDTDASDRLCYAVISSDPTLAPTTSEPEVTQFSMYANNAPYAYAGENRTLVYGLTLTPKEEYYKLGSYYQITDYQRKLVKAFEALD

SEQ ID NO: 25 - protein, 310 aa, Streptomyces sp.
MEEPMSEATEVDRVYAAVEKAAALAGTSCAGDKVLPVLTGHQDLLDDAVIVFSMTASASRSGGLDLSMTVPSGHTDPYSFALSKGLIEPTDHPAGSVVSDFQERFPIGMYGIDVDVAEGFKKAYVAFPSDDLRELDKLVDLPSMPRSAAENAELFTRYGLDKVTGVSVDYKRRELNLYCDLTDGEPMESELVQSMLREMGLKEATEQGLDFAKRSFAVYPTLSWDSSRIERICFAVITTDASTTPTKSEPEAGQMWDYATTAPYAYVGEQRALVYGLALSSEKEYYKLGAYYQISDYQRKLVKAFDSLPE

SEQ ID NO: 26 - protein, 310 aa, Streptomyces sp.
MEEPMSNAPEVDRVYAAVEKAAALAGTACAADKVRPVLSGHQDLLDESVLVFSMTAGGRHGGGLDLSMVVPADHVDPYSFALSNGLIEPTDHPAGSVVSDFKERFPVGMYGIDIDVAGGFKKAYAAFPSDDLRELKHLVDLPSMPRSLAENAELLARYGLDTVTGVSVDYKRHEVNLYCDRATTEPLDPDHVRSMLREMGLREASEQGLEFAKKTFAIYPTLNWDSSKIVRICFAVITNDPATAPTTSEPEAGQMREYATTAPYAYVGERRALVYGLALSPEKEYYKLGAYYQISDYQRKLVKAFDALQD

SEQ ID NO: 27 - protein, 302 aa, Streptomyces sp.
MSETAELTKLYSIIEKTAQVVDVTASRDKVQPILQAFQDVFGQSVISFRASTGRTSSEELDCRFTMLPKGFDPYARALEHGLTPKQDHPVGTLLKEVHQELPIDSCGVDFGVVGGFAKTWSFPSAANLLSISQLTELPSIPGGVAENLDFFKKYGLDDIVSTVGIDYTNRTMNLYFGAGEHRCRPNVSRAKGVKAILKECGLPEPSEELLKLAERAFSIYITMNWDSPKILRVSYAAMTPKPRSLAVKMAPAFDQLLNNAPYSTEGHNFVYGIAATPKGEYHKIASYYQWQTRVEGLLHSES

SEQ ID NO: 28 - protein, 307 aa, Artificial Sequence
  Description of Artificial Sequence Synthetic polypeptide
MSGAADVERVYAAMEEAAGLLGVTCAREKIYPLLTEFQDTLTDGGSVVVFSMASGRRSTELDFSISVPTSQGDPYATVVDKGLFPATGHPVDDLLADTQKHLPVSMFAIDGEVTGGFKKTYAFFPTDDMPGVAQLSAIPSMPSSVAENAELFARYGLDKVHMTSMDYKKRQVNLYFSELSEQTLAPESVLALVRELGLHVPTELGLEFCKRSFSVYPTLNWDTGKIDRLCFAVISTDPTLVPSTDERDIEQFRHYGTKAPYAYVGENRTLVYGLTLSPTEEYYKLGAYYHITDIWRRLLKAFDALED

SEQ ID NO: 29 - protein, 307 aa, Artificial Sequence
  Description of Artificial Sequence Synthetic polypeptide
MSGAADVERVYAAMEEAAGLLGVTCAREKIYPLLTEFQDTLTDGGSVVVFSMASGRRSTELDFSISVPTSQGDPYATVVDKGLFPATGHPVDDLLADTQKHLPVSMFAIDGEVTGGFKKTYAFFPTDDMPGVAQLSAIPSMPSSVAENAELFARYGLDKVSMTSMDYKKRQVNLYFSELSEQTLAPESVLALVRELGLHVPTELGLEFCKRSFSVYPTLNWDTGKIDRLCFAVISTDPTLVPSTDERDIEQFRHYGTKAPYAYVGENRTLVYGLTLSPTEEYYKLGAYYHITDIWRRLLKAFDALED

SEQ ID NO: 30 - protein, 305 aa, Artificial Sequence
  Description of Artificial Sequence Synthetic polypeptide
MSGAADVERVYAAMEEAAGLLGVTCAREKIYPLLTEFQDTLTDGVVVFSMASGRRSTELDFSISVPTSQGDPYATVVDKGLFPATGHPVDDLLADTQKHLPVSMFAIDGEVTGGFKKTYAFFPTDDMPGVAQLSAIPSMPSSVAENAELFARYGLDKVSMTSMDYKKRQVNLYFSELSEQTLAPESVLALVRELGLHVPTELGLEFCKRSFHVYPTLNWDTGKIDRLCFAVISTDPTLVPSTDERDIEQFRHYGTKAPYAYVGENRTLVYGLTLSPTEEYYKLGAYYHITDIQRRLLKAFDALED

SEQ ID NO: 31 - protein, 305 aa, Artificial Sequence
  Description of Artificial Sequence Synthetic polypeptide
MSGAADVERVYAAMEEAAGLLGVTCAREKIYPLLTEFQDTLTDGVVVFSMASGRRSTELDFSISVPTSQGDPYATVVDKGLFPATGHPVDDLLADTQKHLPVSMFAIDGEVTGGFKKTYAFFPTDDMPGVAQLSAIPSMPSSVAENAELFARYGLDKVSMTSMDYKKRQVNLYFSELSEQTLAPESVLALVRELGLHVPTELGLEFCKRSFSVYPTLNWDTGKIDRLCFAVISTDPTLVPSTDERDIEQFRHYGTKAPYAYVGENRTLVYGLTLSPTEEYYKLGAVYHITDIQRRLLKAFDALED

SEQ ID NO: 32 - protein, 305 aa, Artificial Sequence
  Description of Artificial Sequence Synthetic polypeptide
MSGAADVERVYAAMEEAAGLLGVTCAREKIYPLLTEFQDTLTDGVVVFSMASGRRSTELDFSISVPTSQGDPYATVVDKGLFPATGHPVDDLLADTQKHLPVSMFAIDGEVTGGFKKTYAFFPTDDMPGVAQLSAIPSMPSSVAENAELFARYGLDKVQMTSMDYKKRQVNLYFSELSEQTLAPESVLALVRELGLHVPTELGLEFCKRSFHVYPTLNWDTGKIDRLCFAVISTDPTLVPSTDERDIEQFRHYGTKAPYAYVGENRTLVYGLTLSPTEEYYKLGAVYHITDIQRRLLKAFDALED

SEQ ID NO: 33 - protein, 307 aa, Artificial Sequence
  Description of Artificial Sequence Synthetic polypeptide
MSGAADVERVYAAMEEAAGLLGVTCAREKIYPLLTEFQDTLTDGGSVVVFSMASGRRSTELDFSISVPTSQGDPYATVVDKGLFPATGHPVDDLLADTQKHLPVSMFAIDGEVTGGFKKTYAFFPTDDMPGVAQLSAIPSMPSSVAENAELFARYGLDKVSMTSMDYKKRQVNLYFSELSEQTLAPESVLALVRELGLHVPTELGLEFCKRSFSVYPTLNWDTGKIDRLCFAVISTDPTLVPSTDERDIEQFRHYGTKAPYAYVGENRTLVYGLTLSPTEEYYKLGAYYHITDIQRRLLKAFDALED

SEQ ID NO: 34 - protein, 307 aa, Artificial Sequence
  Description of Artificial Sequence Synthetic polypeptide
MSGAADVERVYAAMEEAAGLLGVTCAREKIYPLLTEFQDTLTDGGSVVVFSMASGRRSTELDFSISVPTSQGDPYATVVDKGLFPATGHPVDDLLADTQKHLPVSMFAIDGEVTGGFKKTYAFFPTDDMPGVAQLSAIPSMPSSVAENAELFARYGLDKVQMTSMDYKKRQVNLYFSELSEQTLAPESVLALVRELGLHVPTELGLEFCKRSFHVYPTLNWDTGKIDRLCFAVISTDPTLVPSTDERDIEQFRHYGTKAPYAYVGENRTLVYGLTLSPTEEYYKLGAYYHITDIQRRLLKAFDALED

SEQ ID NO: 35 - protein, 307 aa, Artificial Sequence
  Description of Artificial Sequence Synthetic polypeptide
MSGAADVERVYAAMEEAAGLLGVTCAREKIYPLLTEFQDTLTDGGSVVVFSMASGRRSTELDFSISVPTSQGDPYATVVDKGLFPATGHPVDDLLADTQKHLPVSMFAIDGEVTGGFKKTYAFFPTDDMPGVAQLSAIPSMPSSVAENAELFARYGLDKVQMTSMDYKKRQVNLYFSELSEQTLAPESVLALVRELGLHVPTELGLEFCKRSFSVYPTLNWDTGKIDRLCFAVISTDPTLVPSTDERDIEQFRHYGTKAPYAYVGENRTLVYGLTLSPTEEYYKLGAVYHITDIQRRLLKAFDALED

SEQ ID NO: 36 - protein, 305 aa, Artificial Sequence
  Description of Artificial Sequence Synthetic polypeptide
MSGAADVERVYAAMEEAAGLLGVTCAREKIYPLLTEFQDTLTDGVVVFSMASGRRSTELDFSISVPTSQGDPYATVVDKGLFPATGHPVDDLLADTQKHLPVSMFAIDGEVTGGFKKTYAFFPTDDMPGVAQLSAIPSMPSSVAENAELFARYGLDKVHMTSMDYKKRQVNLYFSELSEQTLAPESVLALVRELGLHVPTELGLEFCKRSFSVYPTLNWDTGKIDRLCFAVISTDPTLVPSTDERDIEQFRHYGTKAPYAYVGENRTLVYGLTLSPTEEYYKLGAYYHITDIWRRLLKAFDALED

SEQ ID NO: 37 - protein, 307 aa, Artificial Sequence
  Description of Artificial Sequence Synthetic polypeptide
MSGAADVERVYAAMEEAAGLLGVTCAREKIYPLLTEFQDTLTDGGSVVVFSMASGRRSTELDFSISVPTSQGDPYATVVDKGLFPATGHPVDDLLADTQKHLPVSMFAIDGEVTGGFKKTYAFFPTDDMPGVAQLSAIPSMPSSVAENAELFARYGLDKVHMTSMDYKKRQVNLYFSELSEQTLAPESVLALVRELGLHVPTELGLEFCKRSFSVYPTLNWDTGKIDRLCFAVISTDPTLVPSTDERDIEQFRHYGTKAPYAYVGENRTLVYGLTLSPTEEYYKLGAYYHITDIQRRLLKAFDALED

SEQ ID NO: 38 - protein, 307 aa, Artificial Sequence
  Description of Artificial Sequence Synthetic polypeptide
MSGAADVERVYAAMEEAAGLLGVTCAREKIYPLLTEFQDTLTDGGSVVVFSMASGRRSTELDFSISVPTSQGDPYATVVDKGLFPATGHPVDDLLADTQKHLPVSMFAIDGEVTGGFKKTYAFFPTDDMPGVAQLSAIPSMPSSVAENAELFARYGLDKVQMTSMDYKKRQVNLYFSELSEQTLAPESVLALVRELGLHVPTELGLEFCKRSFSVYPTLNWDTGKIDRLCFAVISTDPTLVPSTDERDIEQFRHYGTKAPYAYVGENRTLVYGLTLSPTEEYYKLGAYYHITDIWRRLLKAFDALED

SEQ ID NO: 39 - protein, 305 aa, Artificial Sequence
  Description of Artificial Sequence Synthetic polypeptide
MSGAADVERVYAAMEEAAGLLGVTCAREKIYPLLTEFQDTLTDGVVVFSMASGRRSTELDFSISVPTSQGDPYATVVDKGLFPATGHPVDDLLADTQKHLPVSMFAIDGEVTGGFKKTYAFFPTDDMPGVAQLSAIPSMPSSVAENAELFARYGLDKVSMTSMDYKKRQVNLYFSELSEQTLAPESVLALVRELGLHVPTELGLEFCKRSFHVYPTLNWDTGKIDRLCFAVISTDPTLVPSTDERDIEQFRHYGTKAPYAYVGENRTLVYGLTLSPTEEYYKLGAVYHITDIQRRLLKAFDALED

SEQ ID NO: 40 - protein, 307 aa, Artificial Sequence
  Description of Artificial Sequence Synthetic polypeptide
MSGAADVERVYAAMEEAAGLLGVTCAREKIYPLLTEFQDTLTDGGSVVVFSMASGRRSTELDFSISVPTSQGDPYATVVDKGLFPATGHPVDDLLADTQKHLPVSMFAIDGEVTGGFKKTYAFFPTDDMPGVAQLSAIPSMPSSVAENAELFARYGLDKVSMTSMDYKKRQVNLYFSELSEQTLAPESVLALVRELGLHVPTELGLEFCKRSFHVYPTLNWDTGKIDRLCFAVISTDPTLVPSTDERDIEQFRHYGTKAPYAYVGENRTLVYGLTLSPTEEYYKLGAVYHITDIQRRLLKAFDALED

SEQ ID NO: 41 - protein, 307 aa, Artificial Sequence
  Description of Artificial Sequence Synthetic polypeptide
MSGAADVERVYAAMEEAAGLLGVTCAREKIYPLLTEFQDTLTDGGSVVVFSMASGRRSTELDFSISVPTSQGDPYATVVEKGLFPATGHPVDDLLADTQKHLPVSMFAIDGEVTGGFKKTYAFFPTDDMPGVAQLSAIPSMPSSVAENAELFARYGLDKVHMTSMDYKKRQVNLYFSELSEQTLAPESVLALVRELGLHVPTELGLEFCKRSFSVYPTLNWDTGKIDRLCFAVISTDPTLVPSTDERDIEQFRAYGTKAPYAYVGEKRTLVYGLTLSPTEEYYKLGAYYHITDIWRRLLKAFDALED

SEQ ID NO: 42 - protein, 307 aa, Artificial Sequence
  Description of Artificial Sequence Synthetic polypeptide
MSGAADVERVYAAMEEAAGLLGVTCAREKIYPLLTEFQDTLTDGGSVVVFSMASGRRSTELDFSISVPTSQGDPYATVVEKGLFPATGHPVDDLLADTQKHLPVSMFAIDGEVTGGFKKTYAFFPTDDMPGVAQLSAIPSMPSSVAENAELFARYGLDKVSMTSMDYKKRQVNLYFSELSEQTLAPESVLALVRELGLHVPTELGLEFCKRSFSVYPTLNWDTGKIDRLCFAVISTDPTLVPSTDERDIEQFRAYGTKAPYAYVGEKRTLVYGLTLSPTEEYYKLGAYYHITDIWRRLLKAFDALED

SEQ ID NO: 43 - protein, 305 aa, Artificial Sequence
  Description of Artificial Sequence Synthetic polypeptide
MSGAADVERVYAAMEEAAGLLGVTCAREKIYPLLTEFQDTLTDGVVVFSMASGRRSTELDFSISVPTSQGDPYATVVEKGLFPATGHPVDDLLADTQKHLPVSMFAIDGEVTGGFKKTYAFFPTDDMPGVAQLSAIPSMPSSVAENAELFARYGLDKVSMTSMDYKKRQVNLYFSELSEQTLAPESVLALVRELGLHVPTELGLEFCKRSFHVYPTLNWDTGKIDRLCFAVISTDPTLVPSTDERDIEQFRAYGTKAPYAYVGEKRTLVYGLTLSPTEEYYKLGAYYHITDIQRRLLKAFDALED

SEQ ID NO: 44 - protein, 305 aa, Artificial Sequence
  Description of Artificial Sequence Synthetic polypeptide
MSGAADVERVYAAMEEAAGLLGVTCAREKIYPLLTEFQDTLTDGVVVFSMASGRRSTELDFSISVPTSQGDPYATVVEKGLFPATGHPVDDLLADTQKHLPVSMFAIDGEVTGGFKKTYAFFPTDDMPGVAQLSAIPSMPSSVAENAELFARYGLDKVSMTSMDYKKRQVNLYFSELSEQTLAPESVLALVRELGLHVPTELGLEFCKRSFSVYPTLNWDTGKIDRLCFAVISTDPTLVPSTDERDIEQFRAYGTKAPYAYVGEKRTLVYGLTLSPTEEYYKLGAVYHITDIQRRLLKAFDALED

SEQ ID NO: 45 - protein, 305 aa, Artificial Sequence
  Description of Artificial Sequence Synthetic polypeptide
MSGAADVERVYAAMEEAAGLLGVTCAREKIYPLLTEFQDTLTDGVVVFSMASGRRSTELDFSISVPTSQGDPYATVVEKGLFPATGHPVDDLLADTQKHLPVSMFAIDGEVTGGFKKTYAFFPTDDMPGVAQLSAIPSMPSSVAENAELFARYGLDKVQMTSMDYKKRQVNLYFSELSEQTLAPESVLALVRELGLHVPTELGLEFCKRSFHVYPTLNWDTGKIDRLCFAVISTDPTLVPSTDERDIEQFRAYGTKAPYAYVGEKRTLVYGLTLSPTEEYYKLGAVYHITDIQRRLLKAFDALED

SEQ ID NO: 46 - protein, 307 aa, Artificial Sequence
  Description of Artificial Sequence Synthetic polypeptide
MSGAADVERVYAAMEEAAGLLGVTCAREKIYPLLTEFQDTLTDGGSVVVFSMASGRRSTELDFSISVPTSQGDPYATVVEKGLFPATGHPVDDLLADTQKHLPVSMFAIDGEVTGGFKKTYAFFPTDDMPGVAQLSAIPSMPSSVAENAELFARYGLDKVSMTSMDYKKRQVNLYFSELSEQTLAPESVLALVRELGLHVPTELGLEFCKRSFSVYPTLNWDTGKIDRLCFAVISTDPTLVPSTDERDIEQFRAYGTKAPYAYVGEKRTLVYGLTLSPTEEYYKLGAYYHITDIQRRLLKAFDALED

SEQ ID NO: 47 - protein, 307 aa, Artificial Sequence
  Description of Artificial Sequence Synthetic polypeptide
MSGAADVERVYAAMEEAAGLLGVTCAREKIYPLLTEFQDTLTDGGSVVVFSMASGRRSTELDFSISVPTSQGDPYATVVEKGLFPATGHPVDDLLADTQKHLPVSMFAIDGEVTGGFKKTYAFFPTDDMPGVAQLSAIPSMPSSVAENAELFARYGLDKVQMTSMDYKKRQVNLYFSELSEQTLAPESVLALVRELGLHVPTELGLEFCKRSFHVYPTLNWDTGKIDRLCFAVISTDPTLVPSTDERDIEQFRAYGTKAPYAYVGEKRTLVYGLTLSPTEEYYKLGAYYHITDIQRRLLKAFDALED

SEQ ID NO: 48 - protein, 307 aa, Artificial Sequence
  Description of Artificial Sequence Synthetic polypeptide
MSGAADVERVYAAMEEAAGLLGVTCAREKIYPLLTEFQDTLTDGGSVVVFSMASGRRSTELDFSISVPTSQGDPYATVVEKGLFPATGHPVDDLLADTQKHLPVSMFAIDGEVTGGFKKTYAFFPTDDMPGVAQLSAIPSMPSSVAENAELFARYGLDKVQMTSMDYKKRQVNLYFSELSEQTLAPESVLALVRELGLHVPTELGLEFCKRSFSVYPTLNWDTGKIDRLCFAVISTDPTLVPSTDERDIEQFRAYGTKAPYAYVGEKRTLVYGLTLSPTEEYYKLGAVYHITDIQRRLLKAFDALED

SEQ ID NO: 49 - protein, 305 aa, Artificial Sequence
  Description of Artificial Sequence Synthetic polypeptide
MSGAADVERVYAAMEEAAGLLGVTCAREKIYPLLTEFQDTLTDGVVVFSMASGRRSTELDFSISVPTSQGDPYATVVEKGLFPATGHPVDDLLADTQKHLPVSMFAIDGEVTGGFKKTYAFFPTDDMPGVAQLSAIPSMPSSVAENAELFARYGLDKVHMTSMDYKKRQVNLYFSELSEQTLAPESVLALVRELGLHVPTELGLEFCKRSFSVYPTLNWDTGKIDRLCFAVISTDPTLVPSTDERDIEQFRAYGTKAPYAYVGEKRTLVYGLTLSPTEEYYKLGAYYHITDIWRRLLKAFDALED

SEQ ID NO: 50 - protein, 307 aa, Artificial Sequence
  Description of Artificial Sequence Synthetic polypeptide
MSGAADVERVYAAMEEAAGLLGVTCAREKIYPLLTEFQDTLTDGGSVVVFSMASGRRSTELDFSISVPTSQGDPYATVVEKGLFPATGHPVDDLLADTQKHLPVSMFAIDGEVTGGFKKTYAFFPTDDMPGVAQLSAIPSMPSSVAENAELFARYGLDKVHMTSMDYKKRQVNLYFSELSEQTLAPESVLALVRELGLHVPTELGLEFCKRSFSVYPTLNWDTGKIDRLCFAVISTDPTLVPSTDERDIEQFRAYGTKAPYAYVGEKRTLVYGLTLSPTEEYYKLGAYYHITDIQRRLLKAFDALED

SEQ ID NO: 51 - protein, 307 aa, Artificial Sequence
  Description of Artificial Sequence Synthetic polypeptide
MSGAADVERVYAAMEEAAGLLGVTCAREKIYPLLTEFQDTLTDGGSVVVFSMASGRRSTELDFSISVPTSQGDPYATVVEKGLFPATGHPVDDLLADTQKHLPVSMFAIDGEVTGGFKKTYAFFPTDDMPGVAQLSAIPSMPSSVAENAELFARYGLDKVQMTSMDYKKRQVNLYFSELSEQTLAPESVLALVRELGLHVPTELGLEFCKRSFSVYPTLNWDTGKIDRLCFAVISTDPTLVPSTDERDIEQFRAYGTKAPYAYVGEKRTLVYGLTLSPTEEYYKLGAYYHITDIWRRLLKAFDALED

SEQ ID NO: 52 - protein, 305 aa, Artificial Sequence
  Description of Artificial Sequence Synthetic polypeptide
MSGAADVERVYAAMEEAAGLLGVTCAREKIYPLLTEFQDTLTDGVVVFSMASGRRSTELDFSISVPTSQGDPYATVVEKGLFPATGHPVDDLLADTQKHLPVSMFAIDGEVTGGFKKTYAFFPTDDMPGVAQLSAIPSMPSSVAENAELFARYGLDKVSMTSMDYKKRQVNLYFSELSEQTLAPESVLALVRELGLHVPTELGLEFCKRSFHVYPTLNWDTGKIDRLCFAVISTDPTLVPSTDERDIEQFRAYGTKAPYAYVGEKRTLVYGLTLSPTEEYYKLGAVYHITDIQRRLLKAFDALED

SEQ ID NO: 53 - protein, 307 aa, Artificial Sequence
  Description of Artificial Sequence Synthetic polypeptide
MSGAADVERVYAAMEEAAGLLGVTCAREKIYPLLTEFQDTLTDGGSVVVFSMASGRRSTELDFSISVPTSQGDPYATVVEKGLFPATGHPVDDLLADTQKHLPVSMFAIDGEVTGGFKKTYAFFPTDDMPGVAQLSAIPSMPSSVAENAELFARYGLDKVSMTSMDYKKRQVNLYFSELSEQTLAPESVLALVRELGLHVPTELGLEFCKRSFHVYPTLNWDTGKIDRLCFAVISTDPTLVPSTDERDIEQFRAYGTKAPYAYVGEKRTLVYGLTLSPTEEYYKLGAVYHITDIQRRLLKAFDALED

SEQ ID NO: 54 - protein, 307 aa, Artificial Sequence
  Description of Artificial Sequence Synthetic polypeptide
MSGAADVERVYAAMEEAAGLLGVTCAREKIYPLLTEFQDTLTDGGSVVVFSMASGRRSTELDFSISVPTSQGDPYATVVDKGLFPATGHPVDDLLADTQKHLPVSMFAIDGEVTGGFKKTYAFFPTDDMPGVAQLSAIPSMPSSVAENAELFARYGLDKVQMTSMDYKKRQVNLYFSELSEQTLAPESVLALVRELGLHVPTELGLEFCKRSFSVYPTLNWDTGKIDRLCFAVISTDPTLVPSTDERDIEQFRHYGTKAPYAYVGENRTLVYGLTLSPTEEYYKLGAYYHITDIQRRLLKAFDALED

SEQ ID NO: 55 - protein, 307 aa, Artificial Sequence
  Description of Artificial Sequence Synthetic polypeptide
MSGAADVERVYAAMEEAAGLLGVTCAREKIYPLLTEFQDTLTDGGSVVVFSMASGRRSTELDFSISVPTSQGDPYATVVEKGLFPATGHPVDDLLADTQKHLPVSMFAIDGEVTGGFKKTYAFFPTDDMPGVAQLSAIPSMPSSVAENAELFARYGLDKVQMTSMDYKKRQVNLYFSELSEQTLAPESVLALVRELGLHVPTELGLEFCKRSFSVYPTLNWDTGKIDRLCFAVISTDPTLVPSTDERDIEQFRAYGTKAPYAYVGEKRTLVYGLTLSPTEEYYKLGAYYHITDIQRRLLKAFDALED